(19) United States
(12) Patent Application Publication
(10) Pub. No.: US 2013/0204157 A1
(43) Pub. Date: Aug. 8, 2013

(54) CONTACT SENSORS, FORCE/PRESSURE SENSORS, AND METHODS FOR MAKING SAME

(71) Applicant: Sensortech Corporation, Pendleton, SC (US)

(72) Inventors: Andrew C. Clark, Greenville, SC (US); David W. Topham, Senaca, SC (US)

(73) Assignee: Sensortech Corporation, Pendleton, SC (US)

(21) Appl. No.: 13/646,345

(22) Filed: Oct. 5, 2012

Related U.S. Application Data
(63) Continuation of application No. PCT/US2011/031610, filed on Apr. 7, 2011.
(60) Provisional application No. 61/321,734, filed on Apr. 7, 2010.
(60) Provisional application No. 61/371,920, filed on Aug. 9, 2010.

(51) Int. Cl. A61B 5/103; A61B 5/053; A61B 5/00; G01L 1/20

(52) U.S. Cl. CPC A61B 5/1036; G01L 1/20; A61B 5/0538; A61B 5/4585; A61B 5/4571; A61B 5/4576; A61B 5/4595; A61B 5/4566; A61B 5/6878
USPC 600/547; 73/862.68

(57) ABSTRACT

Disclosed herein are contact sensors having a conductive composite material formed of a polymer and a conductive filler. In one particular aspect, the composite materials can include less than about 10 wt % conductive filler. The composite material of the contact sensors can have physical characteristics essentially identical to the polymer, while being electrically conductive with the electrical resistance proportional to the load on the sensor. Also disclosed herein are novel force/pressure sensors that include conductive polymer elements.

### CROSS-REFERENCE TO RELATED APPLICATIONS

The present invention is a continuation application filing under 35 U.S.C. 111(a), which continuation application claims priority to International Application No. PCT/US2011/031610, filed Apr. 7, 2011, which claims the benefit of priority to U.S. Provisional Patent Application Ser. No. 61/321,734, filed on Apr. 7, 2010, and U.S. Provisional Patent Application Ser. No. 61/371,920, filed on Aug. 9, 2010, the entire disclosures of which are incorporated by reference herein for all purposes.

### BACKGROUND OF THE INVENTION

1. Field of the Invention

This invention relates to contact sensors, and more particularly to contact sensors for accurately measuring surface contact data at a junction between two members. This invention also relates to force/pressure sensors, and, more particularly, to force/pressure sensors for accurately measuring both dynamic and static loads.

2. Description of the Related Art

Force/pressure sensors are used in various situations where it is necessary to measure a force exerted on an object or a surface. An exemplary force/pressure sensor is a load cell which is conventionally a transducer that converts force into a measurable electrical output. There are many varieties of load cells, of which strain gage based load cells are the most commonly used type.

Mechanical scales can weigh most objects fairly accurately and reliably if they are properly calibrated and maintained. The method of operation can involve either the use of a weight balancing mechanism or the detection of the force developed by mechanical levers. Other types of force sensors included hydraulic and pneumatic designs. In 1843, English physicist Sir Charles Wheatstone devised a bridge circuit that could measure electrical resistances. The Wheatstone bridge circuit is used for measuring the resistance changes that occur in strain gages. Strain gage load cells are currently the predominate load cell in the weighing industry. Pneumatic load cells are sometimes used where intrinsic safety and hygiene are desired, and hydraulic load cells are considered in remote locations, as they do not require a power supply.

Hydraulic load cells are force-balance devices, measuring weight as a change in pressure of the internal filling fluid. In a rolling diaphragm type hydraulic load cell, a load or force acting on a loading head is transferred to a piston that in turn compresses a filling fluid confined within an elastomeric diaphragm chamber. As force increases, the pressure of the hydraulic fluid rises. This pressure can be locally indicated or transmitted for remote indication or control. Output is linear and relatively unaffected by the amount of the filling fluid or by its temperature. Typical hydraulic load cell applications include tank, bin, and hopper weighing.

Pneumatic load cells also operate on the force-balance principle. These devices use multiple dampener chambers to provide higher accuracy than can a hydraulic device. Pneumatic load cells are often used to measure relatively small weights in industries where cleanliness and safety are of prime concern. The advantages of this type of load cell include their being inherently explosion proof and insensitive to temperature variations. Additionally, they contain no fluids that might contaminate the process if the diaphragm ruptures. Disadvantages include relatively slow speed of response and the need for clean, dry, regulated air or nitrogen.

Strain-gage load cells convert the load acting on them into electrical signals. The gauges themselves are bonded onto a beam or structural member that deforms when weight is applied. In most cases, four strain gages are used to obtain maximum sensitivity and temperature compensation. Two of the gauges are usually in tension, and two in compression, and are wired with compensation adjustments. When weight is applied, the strain changes the electrical resistance of the gauges in proportion to the load.

Contact sensors have been used to gather information concerning contact or near-contact between two surfaces in medical applications, such as dentistry, podiatry, and in the development of prostheses, as well as in industrial applications, such as determinations of load and uniformity of pressure between mating surfaces and development of bearings and gaskets. In general, these sensors include pressure-sensitive films designed to be placed between mating surfaces. These film sensors, while generally suitable for examining static contact characteristics between two generally flat surfaces, have presented many difficulties in other situations. For example, when examining contact data between more complex surfaces, including, for example, surfaces with complex curvatures, for example, it can be difficult to conform the films to fit the surfaces without degrading the sensor's performance.

More serious problems exist with these materials as well. For example, film-based contact sensor devices and methods introduce a foreign material having some thickness between the mating surfaces, which can change the contact characteristic of the junction and overestimate the contact areas between the two surfaces. Moreover, the ability to examine real time, dynamic contact characteristics is practically non-existent with these types of sensors.

A better understanding of the contact conditions at joints and junctions could lead to reduced wear in materials, better fit between mating surfaces, and longer life expectancy for machined parts. For example, one of the leading causes of failure in total joint replacement prostheses is loosening of the implant induced by wear debris particles worn from the polymeric bearing component. A better understanding of the contact conditions between the joint components would lead to reduced implant wear and longer implant life.

A leading cause of wear and revision in prosthetics such as knee implants, hip implants and shoulder implants is less than optimum implant alignment. In a Total Knee Arthroplasty (TKA) procedure, for example, current instrument design for resection of bone limits the alignment of the femoral and tibial resections to average values for varus/valgus flexion/extension and external/internal rotation. Additionally, surgeons often use visual landmarks or “rules of thumb” for alignment which can be misleading due to anatomical variability. While the success rate of the TKA procedure has improved tremendously over the past several decades, revision is still required in a significant number of these cases. About 22,000 of these replacements must be revised each year and even more revisions are predicted for other joint revision surgeries.

In a conventional TKA procedure, in order to correctly balance the forces on each side of the implant after the bone resection has been made, the surgeon performs a procedure known as soft tissue balancing, or ligament balancing, where the collateral ligaments of the knee are partially incised to even out the forces. Releasing some of the soft tissue points can change the balance of the knee; however, the multiple options can be confusing for many surgeons. In revision TKA, for example, many of the visual landmarks are no longer present, making alignment and restoration of the joint line difficult. This is one of the most difficult parts of the surgery to reproduce, and currently available products are not sufficient to effectively assist surgeons with this procedure.

These difficulties frequently cause surgeons to unknowingly create TKA misalignment, which is the leading cause of early failure, and which results in pain and suffering for the patient and increases the risks associated with a second surgery to replace the failed joint. Studies have shown that the most sensitive alignment is the varus/valgus tilt of the tibial insert, with an alignment error of as small as 3 degrees being sufficient to cause premature failure of the implant. In a study where the forces on each side of the implant were measured intra-operatively, over 70% were misaligned in the varus/valgus direction.

Accordingly, there is a need in the pertinent art for improved implant selection, positioning, and design, as well as a better understanding of the in vivo forces of the components of the implant as they relate to each other, the bone, and the surrounding soft tissue structures. There is also a need in the pertinent art for improvement in the mechanical and wear characteristics of knee prostheses such that the prostheses may be expected to last a lifetime. There is a further need in the pertinent art for tools with which physicians can perform diagnostics, during surgery, on prostheses implanted within a patient. There is still a further need in the pertinent art for devices, methods and protocols for joint and bone alignment and tracking for preliminary tests during joint replacement surgery. Additionally, there is a need in the pertinent art for conductive polymer contact sensors that can provide more accurate and/or dynamic load information in an inexpensive manner.

### SUMMARY

In one aspect, the present invention is directed to a contact sensor. The sensor includes an electrically conductive composite material comprising a polymer and a conductive filler. Generally, the composite material can include any polymer. In certain aspects, the polymer can be an engineering polymer or a high performance polymer. In one aspect, the composite material can include ultra-high molecular weight polyethylene (UHMWPE). In another aspect, the composite material can include polyphenylene sulfide (PPS). In one aspect, the composite material of the sensors can include between about 0.1% and 20% by weight of a conductive filler. The conductive filler can be any suitable material. For example, in one aspect, the conductive filler can include carbon black.

The contact sensors of the invention can define a contact surface. In one aspect, a contact surface of the contact sensors of the invention can be placed in a static position so as to replicate a surface that can be placed in proximity to a surface of a second member, thereby forming a junction. In particular, the contact surface of the sensors of the invention can replicate the shape and, optionally, the material characteristics of a junction-forming member found in an industrial, medical, or any other useful setting. For example, in one particular aspect, the contact surface of the sensor can include curvature such as that defined by the contact surface of a polymeric bearing portion of an implantable artificial replacement joint such as the polymeric bearing portion of a hip, knee, or shoulder replacement joint. Alternatively, the contact sensors can be thermoformed into a desired three-dimensional shape. For example, the contact sensors can be thermoformed for use as a prosthetic device.

In one aspect, the sensor can be formed entirely of the composite material. In another aspect, the contact sensors of the invention can include one or more discrete regions of the electrically conductive composite material and a non-conductive material. For example, the sensors can include multiple discrete regions of the electrically conductive composite material that can be separated by an intervening nonconductive material, e.g., an intervening polymeric material. In one particular aspect, the intervening polymeric material separating discrete regions of the composite material can include the same polymer as the polymer of the electrically conductive composite material.

In another aspect, the sensor can comprise one or more sensing points. The sensing points can be configured to measure current flow therethrough the sensing point during application of a load. In one aspect, the current flow measured at each sensing point can be transmitted to a data acquisition terminal. In an additional aspect, the data acquisition terminal can transmit a digital output signal indicative of the current flow measurements to a computer having a processor. In a further aspect, the processor can be configured to calculate the load experienced at each respective sensing point using the digital output signal. In this aspect, the computer can be configured to graphically display the loads experienced at the sensing points as a pressure distribution graph. It is contemplated that the pressure distribution graph can be a three-dimensional plot or a two-dimensional intensity plot wherein various colors correspond to particular load values. It is further contemplated that the computer can be configured to display the pressure distribution graph substantially in real-time. In still a further aspect, the computer can be configured to store the load calculations for the plurality of sensing points for future analysis and graphical display.

In one aspect, the electrically conductive composite material can be located at the contact surface of the sensor for obtaining surface contact data. If desired, the sensor can include composite material that can be confined within the sensor, at a depth below the contact surface, in order to obtain internal stress data.

The electrically conductive composite material described herein can, in one particular aspect, be formed by mixing a polymer in particulate form with a conductive filler in particulate form. According to this aspect, in order to completely coat the polymer granules with the granules of the conductive filler, the granule size of the polymer can be at least about two orders of magnitude larger than the granule size of the conductive filler. For example, the average granule size of the polymer can, in one aspect, be between about 50 μm and about 500 μm. The average granule size of the conductive filler can be, for example, between about 10 nm and about 500 nm.

Following a mixing step, the composite conductive material can be formed into the sensor shape either with or without areas of non-conductive material in the sensor, as desired, by, for example, compression molding, RAM extrusion, or injection molding. If desired, a curvature can be formed into the contact surface of the sensor in the molding step or optionally in a secondary forming step such as a machining or cutting step.

During use, the sensors of the invention can be located in association with a member so as to form a contact junction between a surface of the member and the contact surface of the sensor. The sensor can then be placed in electrical communication with a data acquisition terminal, for example via a fixed or unfixed hard-wired or a wireless communication circuit, and data can be gathered concerning contact between the sensor and the member. In one particular aspect, dynamic contact data can be gathered. For example, any or all of contact stress data, internal stress data, load, impact data, lubrication regime data, and/or information concerning wear, such as wear mode information can be gathered.

In another aspect, the disclosed sensors can be integrated with the part that they have been designed to replicate and actually used in the joint in the desired working setting. For example, the contact sensor can gather data while functioning as a bearing of a joint or junction in real time in an industrial, medical, or other working setting.

In one aspect, the disclosed sensors can use similar materials as those found in an artificial knee implant. In this aspect, it is contemplated that the tibial inserts of the knee implant can be formed with at least one sensor. It is contemplated that the tibial insert can be implanted with the knee implant, which provides for operative sensing during and after the implantation procedure, or, optionally, it is contemplated that the tibial insert can be a trial insert. In this latter instance, the trial tibial insert can be inserted so that the soft tissue balancing can be accomplished with active force/pressure feedback on the joint. After the balancing is complete, an implantable tibial insert, of the same dimensions of the trial tibial insert, can replace the trail tibial insert within the implant. In this aspect, the trial tibial inserts comprising the sensing technology described herein are able to quantify the force being applied to each side of the implant, thereby allowing surgeons to carry out the important step of soft tissue balancing more precisely and reducing the rate of early failure of artificial knees joints.

Also presented herein are aspects of a force/pressure sensor, and in various aspects, a load cell. In one exemplary non-limiting aspect, the force/pressure sensor can be a load cell. In this aspect, the load cell can comprises a load cell housing defining an interior cavity. The load cell housing also defines an opening in a first exterior face. In another aspect, the load cell comprises a load member positioned within the interior cavity, where a load knob protrudes out of the opening and above the first exterior face. The load knob, for example, can be connected directly to the load member, or it can be integral with the load member.

In one aspect, the load cell further comprises a first electrode and a second electrode positioned within the interior cavity. In another aspect, a conductive polymer sensor substantially separates the first and second electrodes.

In operation, in one aspect, a power source can be connected to the load cell via the first and second electrodes. The conductive polymer sensor between the two electrodes completes an electrical circuit. When a force is applied to the load knob, the load is transferred to the first and second electrodes and conductive polymer sensor, compressing the conductive polymer sensor. As the force increases, the current flow through the conductive polymer sensor from the first electrode to the second electrode increases. This current flow can be measured by conventional means and converted to engineering units to calculate the load cell output.

Optionally, the force/pressure sensor can comprises a pliable housing defining an interior cavity. In another aspect, the force/pressure sensor can comprise a conductive polymer sensor that is positioned within the interior cavity. In one aspect, the force/pressure sensor further comprises a first electrode and a second electrode positioned on opposing sides of the conductive polymer sensor. In one aspect, it is contemplated that the housing of the force/pressure sensor can be hermetically sealed to prevent fluid or gas intrusion there into the interior cavity of the housing.

In operation, in one aspect, a power source can be connected to the force/pressure sensor via the first and second electrodes. The conductive polymer sensor between the two electrodes completes an electrical circuit. When a force is applied to the pliable housing, the load is transferred to the first and second electrodes and conductive polymer sensor, compressing the conductive polymer sensor. As the force increases, the current flow through the conductive polymer sensor from the first electrode to the second electrode increases. The change in current flow can be converted into an applied force/pressure unit.

It is contemplated that the devices and methodologies described herein are applicable not only for knee repair, reconstruction or replacement surgery, but also repair, reconstruction or replacement surgery in connection with any other joint of the body, as well as any other medical procedure where it is useful to monitor loading on implant surfaces and to display and output data regarding the loads imposed thereon the implantable prosthesis for use in performance of the procedure.

### BRIEF DESCRIPTION OF THE FIGURES

The accompanying drawings, which are incorporated in and constitute a part of this specification, illustrate certain aspects of the instant invention and together with the description, serve to explain, without limitation, the principles of the invention. Like reference characters used therein indicate like parts throughout the several drawings.

FIG. 1 illustrates a simplified, non-limiting block diagram showing select components of an exemplary operating environment for performing the disclosed methods;

FIG. 2 illustrates one aspect of the sensor disclosed herein for obtaining surface contact data of a junction;

FIG. 3 illustrates another aspect of the sensor disclosed herein for obtaining surface contact data of a junction;

FIG. 4 illustrates another aspect of the sensor disclosed herein for obtaining sub-surface contact data of a junction;

FIG. 5 is a photograph of a sensor sheet according to one aspect disclosed herein, illustrating a plurality of dots comprising a conductive filler;

FIG. 6 is a schematic of a contact sensor in operative communication with a data acquisition terminal, and showing a battery operatively coupled to the data acquisition terminal and a computer coupled to the data acquisition terminal via a Wi-Fi transmitter;

FIG. 7A illustrates another aspect of the sensor disclosed herein for obtaining pressure data of a junction, showing two stacked sensor sheets, each sheet having a plurality of spaced conductive stripes, the stacked sensor sheets being oriented substantially perpendicular to each other such that an array of sensing points is formed by the overlapping portions of the conductive stripes of the stacked sensor sheets;

FIG. 7B illustrates another exemplary aspect of a composite sensor sheet for use in a sensor disclosed herein for obtaining pressure data of a junction, each composite sensor sheet having two stacked sheets 50′″, 50″″, each sheet 50′″, 50″″ having a plurality of spaced conductive stripes, the conductive stripe on sheet 50′″ being less conductive than the conductive stripe on the adjoining sheet 50″″, the respective stacked sheets 50′″, 50″″ being oriented substantially parallel to and overlying each other;

FIG. 7C illustrates another aspect of the sensor disclosed herein for obtaining pressure data of a junction, showing two stacked composite sensor sheets as shown in FIG. 4B, the respective stacked composite sensor sheets being oriented substantially perpendicular to each other such that an array of sensing points is formed by the overlapping portions of the conductive stripes of the stacked sensor sheets, the resulting composite structure having four layers of conductive material that vary, layer to layer, from a lower conductivity, to a second and third higher conductivity, back to the lower conductivity;

FIG. 8 is a schematic of an exemplary interface circuitry for the data acquisition terminal;

FIGS. 9A-9C are schematics of exemplary measurement circuitry for the data acquisition terminal;

FIGS. 10A-10D are images of an exemplary sensor sheet that is formed form a plurality of interwoven stripes of conductive and non-conductive material.

FIG. 11 is a cross-sectional view of a sensor filament as described herein.

FIGS. 12A-12B are images of sensing dots as described herein.

FIG. 13 graphically illustrates the stress v. strain curve for exemplary composite conductive materials as described herein;

FIG. 14 graphically illustrates the log of resistance vs. log of the load for three different composite conductive materials as described herein;

FIG. 15 illustrates the log of normalized resistance vs. log of the load for three different composite conductive materials as described herein;

FIG. 16 illustrates the voltage values corresponding to load, position, and resistance of an exemplary composite material;

FIGS. 17A-17D illustrate the kinematics and contact area for exemplary artificial knee implant sensors as described herein with different surface geometries;

FIGS. 18A and 18B graphically illustrate the log of normalized resistance vs. log of the compressive force for two different composite conductive materials as described herein;

FIG. 19 is a photograph of one aspect of an exemplary mold and press used to form sensor sheets as disclosed herein;

FIG. 20 is a partially transparent perspective view of a load cell as presented herein;

FIG. 21 is a partially transparent exploded perspective view of the load cell of FIG. 17;

FIG. 22 is an exploded side elevational view of the load cell of FIG. 20;

FIG. 23 is a partially transparent top plan view of the load cell of FIG. 20;

FIG. 24 is a schematic illustration of simplified electrical circuit for the load cell;

FIG. 25 is a schematic illustration of the conditioning module interconnects;

FIG. 26 is a hysteresis graph, illustrating the correlation between force and output for forces up to 1000 lbs in an exemplary load cell;

FIG. 27 is a hysteresis graph, illustrating the correlation between force and output for forces up to 500 lbs in an exemplary load cell;

FIG. 28 is an output graph, illustrating the correlation to the output of an exemplary load cell and the change in resistance of a conductive polymer sensor as the mechanical load applied to the load cell is increased;

FIG. 29 is a partially exploded perspective view of a load cell, as presented herein, showing a substantially convex bottom portion of a load member and a substantially convex top portion of a first electrode;

FIGS. 30A and 30B are SEM images of a single UHMWPE granule;

FIGS. 31A and 30B are SEM images of carbon black powder including images of primary particles, aggregates, and agglomerations;

FIGS. 32A and 32B are SEM images of a single UHMWPE granule following formation of a powder mixture including 8 wt % carbon black with UHMWPE;

FIG. 33 is a perspective photograph of an exemplary single point force/pressure sensor;

FIGS. 34 and 35 are top elevational photographs of alternative examples of single point force pressure sensors;

FIGS. 36 and 37 illustrate an exemplary schematic for timing the process of data through the A/D converter;

FIG. 38 illustrates an exemplary schematic for a simplified electrical circuit for the load cell;

FIG. 39 is a perspective photograph of an alternative embodiment of a sensor that is configured to act as a pressure switch;

FIG. 40 is a cross-sectional view of the sensor of FIG. 40; and

FIG. 41 is an exemplary schematic for a comparator circuit that is operatively coupled to a sensor configured to act as a pressure switch.

FIG. 42 is a perspective view of one embodiment of a thin membrane sensor, as described herein.

FIG. 43A is partially transparent, cross-sectional perspective view of another embodiment of a thin membrane sensor, as described herein. FIG. 43B is a side view of the thin membrane sensor of FIG. 43A. FIG. 43C is a top view of the thin membrane sensor of FIG. 43A. FIG. 43D is a top perspective view of the thin membrane sensor of FIG. 43A.

FIG. 44 is a cross-sectional schematic diagram of one embodiment of sensor tape, as described herein; and

FIG. 45 illustrates a simplified, non-limiting block diagram showing select components of an exemplary operating environment for performing the disclosed methods.

### DEFINITIONS OF TERMS

For purposes of the present disclosure, the following terms are herein defined as follows:

The term “static position” is intended to refer to the position of a contact surface of a sensor as described herein at which the contact surface is in equilibrium with adjacent elements within a joint or junction. In the static position, the contact surface will be substantially stationary relative to adjacent joint elements such that any variation in the load applied by a joint element to the contact surface will be detected by the sensor. When a contact surface is supported by a substantially rigid material, the contact surface will typically be in equilibrium with the substantially rigid material, and thus be in the static position, upon contact between the contact sensor and the substantially rigid material. However, when a contact surface is supported by a substantially flexible material, the contact surface will typically be in equilibrium, and thus be in the static position, upon the flexible material reaching its maximum deformation resulting from application of a load to the contact surface.

The term “primary particle” is intended to refer to the smallest particle, generally spheroid, of a material such as carbon black.

The term “aggregate” is intended to refer to the smallest unit of a material, and in particular, of carbon black, found in a dispersion. Aggregates of carbon black are generally considered indivisible and are made up of multiple primary particles held together by strong attractive or physical forces.

The term “granule” is also intended to refer to the smallest unit of a material found in a dispersion. However, while a granule can also be an aggregate, such as when considering carbon black, this is not a requirement of the term. For example, a single granule of a polymer, such as UHMWPE or conventional grade polyethylene, for example can be a single unit.

The term “agglomeration” is intended to refer to a configuration of a material including multiple aggregates or granules loosely held together, as with Van der Waals forces. Agglomerations of material in a dispersion can often be broken down into smaller aggregates or granules upon application of sufficient energy so as to overcome the attractive forces.

The term “conventional polymer” is intended to refer to polymers that have a thermal resistance below about 100° C. and relatively low physical properties. Examples include high-density polyethylene (PE), polystyrene (PS), polyvinyl chloride (PVC), and polypropylene (PP).

The term “engineering polymer” is intended to refer to polymers that have a thermal resistance between about 100° C. and about 150° C. and exhibit higher physical properties, such as strength and wear resistance, as compared to conventional polymers. Examples include polycarbonates (PC), polyamides (PA), polyethylene terephthalate (PET), and ultrahigh molecular weight polyethylene (UHMWPE).

The term “high performance polymer” is intended to refer to polymers that have a thermal resistance greater than about 150° C. and relatively high physical properties. Examples include polyetherether ketone (PEEK), polyether sulfone (PES), polyimides (PI), and liquid crystal polymers (LC).

Contact stress, synonymous with contact pressure, is herein defined as surface stress resulting from the mechanical interaction of two members. It is equivalent to the applied load (total force applied) divided by the area of contact.

Internal stress refers to the forces acting on an infinitely small unit area at any point within a material. Internal stress varies throughout a material and is dependent upon the geometry of the member as well as loading conditions and material properties.

Impact force is herein defined to refer to the time-dependent force one object exerts onto another object during a dynamic collision.

### DETAILED DESCRIPTION

The present invention can be understood more readily by reference to the following detailed description, examples, and claims, and their previous and following description. Before the present system, devices, and/or methods are disclosed and described, it is to be understood that this invention is not limited to the specific systems, devices, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. Those skilled in the relevant art will recognize that many changes can be made to the aspects described, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used herein, the singular forms “a,” “an” and “the” include plural referents unless the context clearly dictates otherwise. Thus, for example, reference to a “sensor” includes aspects having two or more sensors unless the context clearly indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes examples where said event or circumstance occurs and examples where it does not.

### Contact Sensors

Presented herein are contact sensors, methods of forming contact sensors, and methods of advantageously utilizing the sensors. In general, contact sensors can be utilized to gather dynamic and/or static contact data at the junction of two opposing members such as a junction found in a joint, a bearing, a coupling, a connection, or any other junction involving the mechanical interaction of two opposing members, and including junctions with either high or low tolerance values as well as junctions including intervening materials between the members, such as lubricated junctions, for example. Dynamic and/or static data that can be gathered utilizing the disclosed sensors can include, for example, load data, lubrication regimes, wear modes, contact stress data, internal stress data, and/or impact data for a member forming the junction. The contact sensors disclosed herein can provide extremely accurate data for the junction being examined, particularly in those aspects wherein at least one of the members forming the junction in the working setting (as opposed, for example, to a testing setting) is formed of a polymeric material.

Beneficially, the sensors described herein can be configured to replicate either one of the mating surfaces forming the junction. Optionally, the sensors described herein can be essentially inflexible when positioned proximate the junction. As such, in a laboratory-type testing application, the sensor can simulate one member forming the junction, and contact data can be gathered for the junction under conditions closer to those expected during actual use, i.e., without altering the expected contact dynamics experienced at the junction during actual use. For example, the disclosed sensors can provide contact data for the junction without the necessity of including extraneous testing material, such as dyes, thin films, or the like, within the junction itself.

In one aspect, the sensor can be formed of a material that essentially duplicates the physical characteristics of the junction member that the sensor is replicating. Accordingly, in this aspect, the sensor can exhibit wear characteristics essentially equivalent to those of the member when utilized in the field, thereby improving the accuracy of the testing data. According to one particular aspect of the invention, rather than being limited to merely simulating a junction-forming member, such as in a pure testing situation, the sensor can be incorporated into the member itself that is destined for use in the working application, i.e., in the field, and can provide contact data for the junction during actual use of the part. It is contemplated that the sensors described herein can be used in a variety of working settings, including, for example and without limitation, in industrial working settings, medical working settings, and the like.

In an additional aspect, the contact sensors disclosed herein can be formed to be substantially non-deformable. Alternatively, the contact sensors disclosed herein can be formed to be substantially deformable. It is further contemplated that the contact sensors can be thermoformed as desired into a three-dimensional shape. In one aspect, it is contemplated that the desired shape of the contact sensors can be a substantially sheet-like member. Optionally, the desired shape of the contact sensors can substantially replicate the three-dimensional shape of a selected structure of a subject's body, including, for example and without limitation, a bone, limb, or other body member. Accordingly, it is contemplated that the contact sensors can optionally be thermoformed to function, for example and without limitation, as prosthetic devices for use as a replacement for, or in conjunction with, the selected structure of the subject's body. It is further contemplated that the desired shape of the contact sensors can substantially replicate the three-dimensional shape of a selected structure outside the body of a subject that is configured to bear loads, including, for example and without limitation, textile devices, vehicle parts and components, anthropomorphic test devices such as crash test dummies, building components, and the like.

In exemplary aspects, it is further contemplated that the contact sensors can optionally be selectively returnable to a substantially flat configuration following bending of the contact sensors to arrive at a desired three-dimensional shape. In these aspects, it is contemplated that the contact sensors can be selectively bent into the desired three-dimensional shape and then selectively flexed to return the contact sensors to their original, substantially flat configuration.

In a further aspect, during use of the contact sensors disclosed herein, the contact sensors can be configured to measure a load upon positioning of the contact surface of each contact sensor in a static position. In one aspect, the contact sensors disclosed herein can be formed to be substantially pliable. In this aspect, it is contemplated that the static position can correspond to the contact sensors contacting or abutting a substantially rigid material such that the contact surface of each contact sensor is placed in the static position. For example, a contact sensor can be positioned therebetween two or more substantially rigid conductive elements as described herein such that the contact sensor is in the static position. In another example, the contact sensor can be attached to a substantially rigid insert such that the contact surface is in the static position when the insert is inserted therebetween two or more conductive elements. Alternatively, in another aspect, the contact sensor can be attached to or abut one or more flexible elements as described herein, and the static position can correspond to a state of equilibrium between the elements of a joint, including the contact sensor and the one or more flexible elements. Thus, upon application of a load to a contact surface of a contact sensor within a joint, the contact surface will be placed in the static position when a state of equilibrium is reached within the joint such that the contact surface is substantially stationary relative to adjacent surfaces of other elements of the joint.

In an additional aspect, the contact sensors disclosed herein can be formed to be substantially unpliable. In this aspect, it is contemplated that the static position can correspond to placement of the substantially unpliable contact sensors in any operative position such that the contact sensors can be used as disclosed herein.

As contemplated, in one aspect, changes in resistivity of the contact surface are being measured to determine the applied load or force on the sensor. More particularly, in one aspect, instead of measuring the changes in bulk resistivity of the material forming the sensor, the resistivity changes at the surface of the sensor due to applied loads are being measured. By surface, it is meant the surface portions of the sensor that extend to a depth of about 50 nm, more preferably to a depth of about 100 nm, and most preferably to a depth of about 1,000 nm.

In various aspects, the contact sensors disclosed herein can comprise an electrically conductive composite material that in turn comprises at least one non-conductive polymer material combined with an electrically conductive filler. In another aspect, the composite material disclosed herein can comprise an electrically conductive filler that can provide pressure sensitive electrical conductivity to the composite material, but can do so while maintaining the physical characteristics, e.g., wear resistance, hardness, etc., of the non-conductive polymeric material of the composite. Thus, in this aspect, the sensors disclosed herein can be developed to include a particular polymer or combination of polymers so as to essentially replicate the physical characteristics of the similar but nonconductive polymeric member forming the junction or three-dimensional structure to be examined.

This combination of beneficial characteristics in the composite materials has been attained through recognition and/or development of processes for forming the composite materials in which only a small amount of the electrically conductive filler need be combined with the polymeric material. As such, the physical characteristics of the composite material can more closely resemble those of the starting polymeric material, and the sensor can closely replicate the physical characteristics of a non-conductive polymeric member forming a junction.

This feature can be particularly beneficial when considering the examination of junctions including at least one member formed of engineering and/or high performance polymers. When considering such materials, the addition of even a relatively small amount of additive or filler can drastically alter the physical characteristics that provide the desired performance of the materials. In the past, when attempts were made to form electrically conductive composites of many engineering and high performance polymers, the high levels of additives (greater than about 20% by weight, in most examples) that were required usually altered the physical characteristics of the polymeric material to the point that the formed conductive composite material no longer exhibited the desired characteristics of the starting, non-conductive material. Thus, the examination of junctions formed with such materials has in the past generally required the addition of an intervening material, such as a pressure sensitive film within the junction, leading to the problems discussed above.

It should be noted, however, that while the presently disclosed sensors can be of great benefit when formed to include engineering and/or high performance polymeric composite materials, this is not a requirement of the invention. In other aspects, the polymer utilized to form the composite material can be a more conventional polymer. Regardless of the polymer, copolymer, or combination of polymers that is used to form the disclosed composite conductive materials, the composite materials of the disclosed sensors can exhibit pressure sensitive electrical conductivity and, if desired, can also be formed so as to essentially maintain the physical characteristics of a polymeric material identical to the composite but for the lack of the conductive filler.

In general, any polymeric material that can be combined with an electrically conductive filler to form a pressure sensitive conductive polymeric composite material can be utilized in the contact sensors described herein. For example, various polyolefins, polyurethanes, polyester resins, epoxy resins, and the like can be utilized in the contact sensors described herein. In certain aspects, the composite material can include engineering and/or high performance polymeric materials. In one particular aspect, the composite material can comprise UHMWPE. UHMWPE is generally classified as an engineering polymer, and possesses a unique combination of physical and mechanical properties that allows it to perform extremely well in rigorous wear conditions. In fact, it has the highest known impact strength of any thermoplastic presently made, and is highly resistant to abrasion, with a very low coefficient of friction. The physical characteristics of UHMWPE have made it attractive in a number of industrial and medical applications. For example, it is commonly used in forming polymeric gears, sprockets, impact surfaces bearings, bushings and the like. In the medical industry, UHMWPE is commonly utilized in forming replacement joints including portions of artificial hips, knees, and shoulders. In addition, UHMWPE can be in particulate form at ambient conditions and can be shaped through compression molding or RAM extrusion and can optionally be machined to form a substantially unpliable block (i.e., not easily misshapen or distorted), with any desired surface shape. In another aspect, the composite material can comprise PPS.

Conductive fillers as are generally known in the art can be combined with the polymeric material of choice to form the composite material of the disclosed sensors. The conductive fillers can be, for example and without limitation, carbon black and other known carbons, gold, silver, aluminum, copper, chromium, nickel, platinum, tungsten, titanium, iron, zinc, lead, molybdenum, selenium, indium, bismuth, tin, magnesium, manganese, cobalt, titanium germanium, mercury, and the like.

According to one aspect, a pressure sensitive conductive composite material can be formed by combining a relatively small amount of a conductive filler with a polymeric material. For example, the composite can comprise from between about 0.1% to about 20% by weight of the conductive filler, more preferably from between about 1% to about 15% by weight of the conductive filler, and most preferably from between about 5% to about 12% by weight of the conductive filler. Of course, in other aspects, such as those in which the physical characteristics of the composite material need not approach those of the non-conductive polymeric material, the composite material can include a higher weight percentage of the conductive filler material.

In general, the polymeric material and the conductive filler can be combined in any suitable fashion, which can generally be determined at least in part according to the characteristics of the polymeric material. For example, and depending upon the polymers involved, the materials can be combined by mixing at a temperature above the melting temperature of the polymer (conventional melt-mixing) and the filler materials can be added to the molten polymer, for example, in a conventional screw extruder, paddle blender, ribbon blender, or any other conventional melt-mixing device. The materials can also be combined by mixing the materials in an appropriate solvent for the polymer (conventional solution-mixing or solvent-mixing) such that the polymer is in the aqueous state and the fillers can be added to the solution. Optionally, an appropriate surfactant can be added to the mixture of materials to permit or encourage evaporation of the solvent, resulting in the solid conductive composite material. In another aspect, the materials can be mixed below the melting point of the polymer and in dry form. In this aspect, the materials can be mixed by a standard vortex mixer, a paddle blender, a ribbon blender, or the like, such that the dry materials are mixed together before further processing.

When mixing the components of the composite material, the mixing can be carried out at any suitable conditions. For example, in one aspect, the components of the composite material can be mixed at ambient conditions. In other aspects, however, the components of the composite material can be mixed at non-ambient conditions. It is contemplated that the components of the composite material can be mixed under non-ambient conditions to, for example and without limitation, maintain the materials to be mixed in the desired physical state and/or to improve the mixing process.

When dry mixing the materials to be utilized in the composite, the exact particulate dimensions of the materials are not generally critical to the invention. However, in certain aspects, the relative particulate size of the materials to be combined in the mixture can be important. In particular, the relative particulate size of the materials to be combined can be important in those aspects wherein a relatively low amount of conductive filler is desired and in those aspects wherein the polymer granules do not completely fluidize during processing. For example, the relative particle size can be important in certain aspects wherein engineering or high-performance polymers are utilized. It is contemplated that the relative particle size can be particularly important during utilization of extremely high melt viscosity polymers such as UHMWPE, which can be converted via non-fluidizing conversion processes, including, for example and without limitation, compression molding or RAM extrusion processes.

In such aspects, the particle size of the filler can beneficially be considerably smaller than the particle size of the polymer. According to this aspect, it is contemplated that due to the small size of the conductive filler particles relative to the larger polymer particles, the conductive filler is able to completely coat the polymer during mixing and, upon conversion of the composite polymeric powder in a non-fluidizing conversion process to the final solid form, the inter-particle distance of the conductive filler particles can remain above the percolation threshold such that the composite material can exhibit the desired electrical conductivity. According to this aspect, when forming the composite mixture, the granule or aggregate size of the conductive filler to be mixed with the polymer can be at least about two orders of magnitude smaller than the granule size of the polymer. In some aspects, the granule or aggregate size of the conductive filler can be at least about three orders of magnitude smaller than the granule size of the polymer.

In forming the composite material according to this aspect, a granular polymer can be dry mixed with a conductive filler that is also in particulate form. Readily available UHMWPE can have a granule diameter in a range of from about 50 μm to about 200 μm. Typically, the individual granule can be made up of multiple sub-micron sized spheroids and nano-sized fibrils surrounded by varying amounts of free space.

In one aspect, the conductive filler for mixing with the polymer can comprise carbon black. Carbon black is readily available in a wide variety of agglomerate sizes, generally having diameters ranging from about 1 μm to about 100 μm. It is contemplated that these agglomerates can be broken down into smaller aggregates having diameters ranging from about 10 nm to about 500 nm upon application of suitable energy.

Upon dry mixing of the particulate conductive filler and the larger particulate polymer material with suitable energy, the smaller granules of conductive filler material can completely coat the larger polymer granules. For example, a single powder particle can be obtained following mixing of 8 wt % carbon black with 92 wt % UHMWPE. It is contemplated that the UHMWPE particles can be completely coated with carbon black aggregates. It is further contemplated that the combination of mixing forces with electrostatic attractive forces existing between the non-conductive polymeric particles and the smaller conductive particles is primarily responsible for breaking the agglomerates of the conductive material down into smaller aggregates and forming and holding the coating layer of the conductive material on the polymer particles during formation of the composite powder, as well as during later conversion of the powdered composite material into a solid form.

Following formation of the mixture comprising the conductive filler and the polymeric material, the mixture can be converted as desired to form a solid composite material. In one aspect, the solid composite material can be electrically conductive. The solid composite material thus formed can also maintain the physical characteristics of the polymeric material in mixtures comprising a relatively low weight percentage of conductive filler. For example, in the aspect described above, in which the composite material includes a conductive filler mixed with UHMWPE, the powder can be converted via a compression molding process or a RAM extrusion process, as is generally known in the art. Optionally, following conversion of the powder, the resultant solid molded material can be machined to produce a desired curvature on at least one contact surface.

In other aspects however, and primarily depending upon the nature of the polymeric portion of the composite, other conversion methods may preferably be employed. For example, in other aspects, the polymeric portion of the composite material can be a polymer, a co-polymer, or a mixture of polymers that can be suitable for other converting processes. For example and without limitation, the composite polymeric material can be converted via a conventional extrusion or injection molding process.

The composite material of the disclosed sensors can optionally comprise other materials in addition to the primary polymeric component and the conductive filler discussed above. In one aspect, the composite material can comprise additional fillers, including, for example and without limitation, various ceramic fillers, aluminum oxide, zirconia, calcium, silicon, fibrous fillers, including carbon fibers and/or glass fibers, or any other fillers as are generally known in the art. In another aspect, the composite material can include an organic filler, including for example and without limitation, tetrafluoroethylene or a fluororesin. In this aspect, it is contemplated that the organic filler can be added to improve sliding properties of the composite material.

It is believed that during the conversion process, the polymer particles can fuse together and confine the conductive filler particles to a three-dimensional channel network within the composite, forming a segregated network type of composite material. In operation, the distance between individual carbon black primary particles and surrounding small aggregates can be about 10 nm. It is contemplated that when two conductive filler particles are within about 10 nm of each other, the conductive filler particles can conduct current via electron tunneling, or percolation, with very little resistance. Thus, many conductive paths fulfilling these conditions can exist within the composite material. Moreover, when deformable polymers are used, the conductivity, and in particular the resistance, of the composite material of the contact sensors described herein can vary upon application of a compressive force (i.e., load) to the composite material.

Accordingly, following any desired molding, shaping, cutting and/or machining and also following any desired physical combination of the formed composite material with other non-conductive materials (various aspects of which are discussed further below), the composite materials of the contact sensors described herein, which comprise at least one conductive filler, can be formed into the sensor shape and placed in electrical communication with a data acquisition terminal. For example, in one aspect, the composite material of the sensor can be connected to a data acquisition terminal. In this aspect, the composite material can be connected to the data acquisition terminal by, for example and without limitation, conventional alligator clips, conductive epoxy, conductive silver ink, conventional rivet mechanisms, conventional crimping mechanisms, and other conventional mechanisms for maintaining electrical connections. In another aspect, the composite material can be machined to accept a connector of a predetermined geometry within the composite material itself. Other connection regimes as are generally known in the art may optionally be utilized, however, including fixed or unfixed connections to any suitable communication system between the composite material and the data acquisition terminal. No particular electrical communication system is required of the contact sensors described herein. For example, in some aspects, the electrical communication between the composite material and the data acquisition terminal can be wireless, rather than a hard wired connection.

In one aspect, the data acquisition terminal can comprise data acquisition circuitry. In another aspect, the data acquisition terminal can comprise at least one multiplexer placed in electrical communication with a microcontroller via the data acquisition circuitry. In an additional aspect, the data acquisition circuitry can comprise at least one op-amp for providing a predetermined offset and gain through the circuitry. In this aspect, the at least one op-amp can comprise a converting op-amp configured to convert a current reading into a voltage output. It is contemplated that the converting op-amp can measure current after it has passed through the at least one multiplexer and then convert the measured current into a voltage output. In a further aspect, the data acquisition terminal can comprise an Analog/Digital (A/D) converter. In this aspect, the A/D converter can be configured to receive the voltage output from the converting op-amp. It is contemplated that the A/D converter can convert the voltage output into a digital output signal. In yet another aspect, the data acquisition terminal can be in electrical communication with a computer having a processor. In this aspect, the computer can be configured to receive the digital output signal from the A/D converter. It is contemplated that the A/D converter can have a conventional Wi-Fi transmitter for wirelessly transmitting the digital output signal to the computer. It is further contemplated that the computer can have a conventional Wi-Fi receiver to receive the digital output signal from the A/D converter.

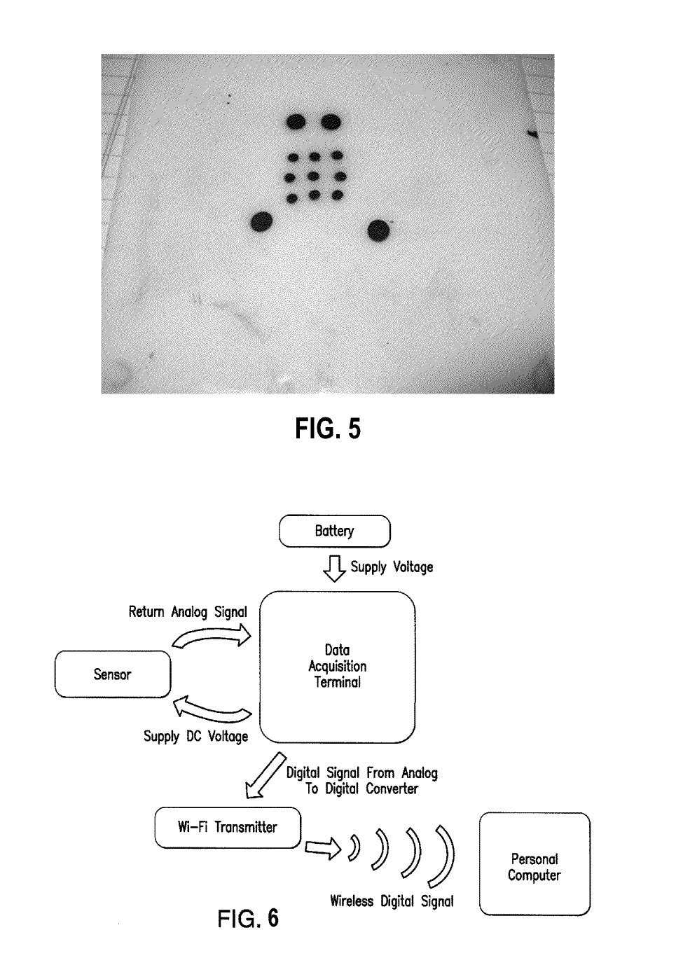

As electrical communications methods and electrical data analysis methods and systems are generally known in the art, these particular aspects of the disclosed contact sensor systems are not described in great detail herein. FIG. 5 is a block diagram illustrating an exemplary operating environment for performing the disclosed methods and portions thereof. This exemplary operating environment is only an example of an operating environment and is not intended to suggest any limitation as to the scope of use or functionality of operating environment architecture. Similarly, the operating environment contemplated for the contact sensors disclosed herein should not be interpreted as having any dependency or requirement relating to any one component or combination of components illustrated in the exemplary operating environment.

The present methods and systems can be operational with numerous other general purpose or special purpose computing system environments or configurations. Examples of well known computing systems, environments, and/or configurations that can be suitable for use with the system and method comprise, but are not limited to, personal computers, server computers, laptop devices, hand-held electronic devices, vehicle-embedded electronic devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components. The disclosed system and method can be described in the general context of computer-executable instructions, such as program modules, being executed by one or more computers or other devices. Generally, program modules comprise computer code, routines, programs, objects, components, data structures, etc. that perform particular tasks or implement particular abstract data types. In one aspect, the program modules can comprise a system control module. The disclosed method can also be practiced in grid-based and distributed computing environments where tasks are performed by remote processing devices that are linked through a communications network. In a distributed computing environment, program modules can be located in both local and remote computer storage media including memory storage devices.

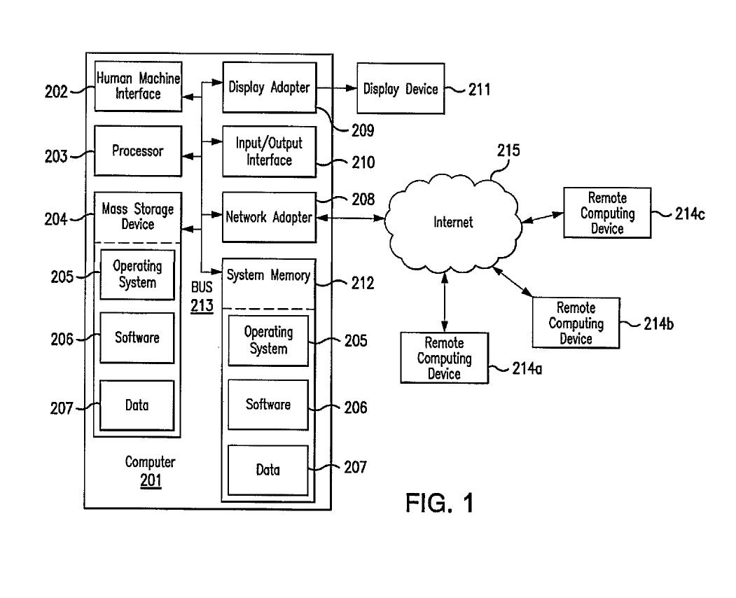

Further, one skilled in the art will appreciate that the system and method disclosed herein can be implemented via a general-purpose computing device in the form of a computer 200. As schematically illustrated in FIG. 1, the components of the computer 200 can comprise, but are not limited to, one or more processors or processing units 203, a system memory 212, and a system bus 213 that couples various system components including the processor 203 to the system memory 212.

The system bus 213 represents one or more of several possible types of bus structures, including a memory bus or memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI) bus also known as a Mezzanine bus. The bus 213, and all buses specified in this description can also be implemented over a wired or wireless network connection and each of the subsystems, including the processor 203, a mass storage device 204, an operating system 205, contact sensor software 206, contact sensor data 207, a network adapter 208, system memory 212, an Input/Output Interface 210, a display adapter 209, a display device 211, and a human machine interface 202, can be contained within one or more remote computing devices 214a,b,c at physically separate locations, connected through buses of this form, in effect implementing a fully distributed system.

The computer 200 typically comprises a variety of computer readable media. Exemplary readable media can be any available media that is accessible by the computer 200 and comprises, for example and without limitation, both volatile and non-volatile media, removable and non-removable media. The system memory 212 can comprise computer readable media in the form of volatile memory, such as random access memory (RAM), and/or non-volatile memory, such as read only memory (ROM). The system memory 212 typically contains data such as pressure and/or hysteresis data 207 and/or program modules such as operating system 205 and contact sensor module software 206 that are immediately accessible to and/or are presently operated on by the processing unit 203.

In another aspect, the computer 200 can also comprise other removable/non-removable, volatile/non-volatile computer storage media. By way of example, FIG. 1 illustrates a mass storage device 204 which can provide non-volatile storage of computer code, computer readable instructions, data structures, program modules, and other data for the computer 200. For example and without limitation, a mass storage device 204 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 204, including by way of example, an operating system 205 and contact sensor module software 206. It is contemplated that both the operating system 205 and the contact sensor module software 206 can comprise at least some elements of the programming. Pressure and/or hysteresis data 207 can also be stored on the mass storage device 204. Pressure and/or hysteresis data 207 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple systems.

In another aspect, the user can enter commands and information into the computer 200 via an input device (not shown). It is contemplated that the input device can comprise, for example and without limitation, a keyboard, pointing device (e.g., a “mouse”), a microphone, a joystick, a scanner, tactile input devices such as gloves and other body coverings, and the like. These and other input devices can be connected to the processing unit 203 via a human machine interface 202 that is coupled to the system bus 213. However, it is contemplated that the input devices can be connected to the processing unit 203 by other interface and bus structures, including, for example and without limitation, a parallel port, game port, an IEEE 1394 Port (also known as a Firewire port), a serial port, and a universal serial bus (USB).

In yet another aspect, a display device 211 can also be connected to the system bus 213 via an interface, such as a display adapter 209. It is contemplated that the computer 200 can have more than one display adapter 209 and the computer 200 can have more than one display device 211. For example, a display device can be a monitor, an LCD (Liquid Crystal Display), or a projector. In addition to the display device 211, other output peripheral devices can comprise components such as a printer (not shown) which can be connected to the computer 200 via Input/Output Interface 210.

The computer 200 can operate in a networked environment using logical connections to one or more remote computing devices 214a,b,c. By way of example, a remote computing device can be a personal computer, portable computer, a server, a router, a network computer, a peer device or other common network node, and the like. Logical connections between the computer 200 and a remote computing device 214a,b,c can be made via a local area network (LAN) and a general wide area network (WAN). Such network connections can be through a network adapter 208. A network adapter 208 can be implemented in both wired and wireless environments. Such networking environments are conventional and commonplace in offices, enterprise-wide computer networks, intranets, and the Internet 215.

For purposes of illustration, application programs and other executable program components such as the operating system 205 are illustrated herein as discrete blocks, although it is recognized that such programs and components reside at various times in different storage components of the computing device 200, and are executed by the data processor(s) of the computer. An implementation of contact sensor software 206 can be stored on or transmitted across some form of computer readable media. Computer readable media can be any available media that can be accessed by a computer. By way of example and not meant to be limiting, computer readable media can comprise “computer storage media” and “communications media.” “Computer storage media” comprise volatile and nonvolatile, removable and non-removable media implemented in any method or technology for storage of information such as computer readable instructions, data structures, program modules, or other data. Exemplary computer storage media can comprise, for example and without limitation, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

In various aspects, it is contemplated that the methods and systems described herein can employ Artificial Intelligence techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. expert inference rules generated through a neural network or production rules from statistical learning).

It is contemplated that the contact sensors described herein can optionally comprise one or more sensing points. In one aspect, the contact sensor can include only a single sensing point. For example, the entire contact surface of the disclosed sensors can be formed of the conductive composite material. According to this aspect, the contact sensors can be utilized to obtain impact data and/or the total load on the contact surface at any time. Such an aspect can be preferred, for example, in order to obtain total load or impact data for a member without the necessity of having external load cells or strain gauges in communication with the load-bearing member. This sensor type may be particularly beneficial in those aspects wherein the sensor is intended to be incorporated with or as the member for use in the field. For example, any polymeric load-bearing member utilized in a process could be formed from the physically equivalent but conductive composite material as described herein and incorporated into the working process to provide real time wear and load data of the member without diminishing the wear performance of the member due to the acquisition of conductive capability.

In other aspects, the sensors disclosed herein can include a plurality of sensing points and can provide more detailed data about the junction or the members forming the junction. For example, the plurality of sensing points can provide data describing the distribution of contact stresses and/or internal stresses, data concerning types of wear modes, or data concerning a lubrication regime as well as load and impact data for a member forming a junction. According to this aspect, the composite material can be located at predetermined, discrete regions of a sensor to form the plurality of sensing points on or in the sensor, and a non-conductive material can separate the discrete sensing points from one another. Data from the plurality of discrete sensing points can then be correlated and analyzed and can provide information concerning, for example, the distribution of contact characteristics across the entire mating surface, and in particular can provide contact information under dynamic loading conditions involving, for example, sliding, rolling, or grinding motions across the surface of the sensor.

It is contemplated that the plurality of sensing points can be arranged in any desired configuration along a surface of the sensor. For example, and without limitation, the sensing points can be positioned in a series of parallel rows. Alternatively, the sensing points can be positioned in staggered or overlapping configurations. In one aspect, the sensing points can be substantially evenly spaced. In another aspect, the sensing points can be substantially unevenly spaced.

It is contemplated that selected sensing points among the plurality of sensing points can be activated during the application of a load while the remainder of the sensing points remain deactivated.

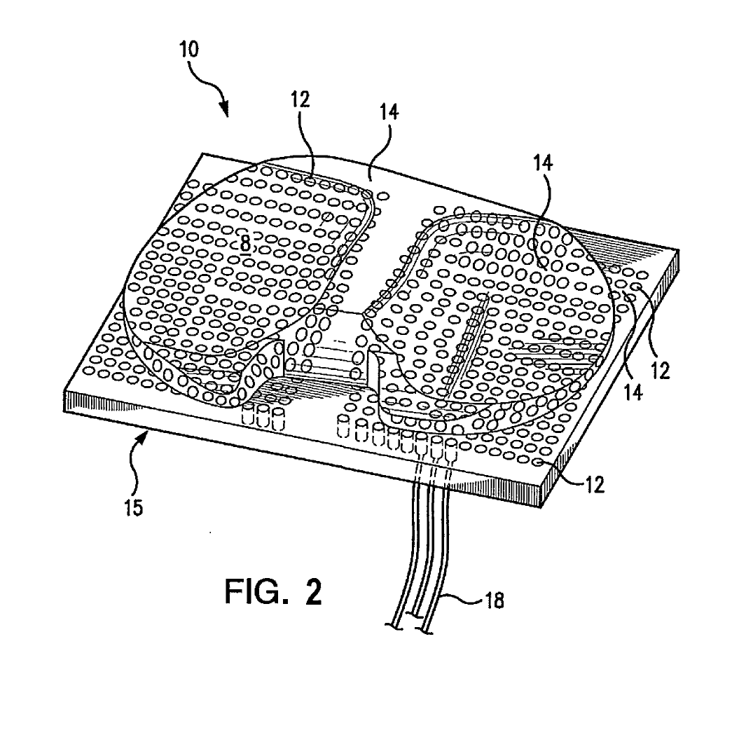

FIG. 2 is a schematic diagram of one aspect of the sensor as disclosed herein, including a plurality of sensing points at the contact surface of the junction member. Surface sensing points such as those in this aspect can be utilized to determine contact surface data, including, for example and without limitation, contact stress data, lubrication data, impact data, and information concerning wear modes. The polymeric sensor 10 includes a contact surface 8 for contact with an electrically conductive joint element (not shown) to simulate the dynamic characteristics of the joint formed between the sensor and the conductive joint element. In this particular aspect, the contact surface 8 defines a curvature to simulate that of the tibial plateau of an artificial knee implant. It is contemplated that the conductive joint element can be metallic.

As can be seen with reference to FIG. 2, the sensor 10 includes a plurality of sensing points 12 at the contact surface 8 of the sensor 10. The sensing points 12 can be formed of the conductive composite material as herein described. Thus, unlike conventional contact sensors, the conductive composite material functions as not only the sensing material, but also as an electrical communication pathway. After a load is applied to the sensor 10 and the contact surface 8 is positioned in the static position, each sensing point 12 is configured to produce an output signal in response to the change in resistance experienced by the conductive composite material at the contact surface proximate the sensing point.

In one aspect, because the conductive composite material provides electrical communication between the sensing points 12 at the contact surface 8 of the sensor 10, the conductive composite material of each sensor can have a bulk resistance. In this aspect, the bulk resistance can be measured in Ohms per unit length; accordingly, as the length of the sensor 10 increases, the bulk resistance proportionally increases. Therefore, the bulk resistance of the conductive composite material varies from one sensing point to another sensing point. It is contemplated that the farther a particular sensing point is from an electrical connection between the sensor 10 and the data acquisition terminal, the greater the bulk resistance will be at that particular sensing point. Consequently, it is contemplated that the resistance measured at each sensing point will be different even when the change in resistance at some sensing points is identical. In addition, it is contemplated that the sensing points can always have at least some level of electrical communication with adjacent sensing points, even when a load is not being applied. Thus, when a load is applied to one or more sensing points, the sensing points that are subjected to the load can generate current within sensing points that are not subjected to the load, thereby creating parallel resistance paths.

In an additional aspect, it is contemplated that the bulk resistance of the conductive composite material can be calibrated to measure current changes. In this aspect, it is further contemplated that, where the measured current changes correspond to known changes in ambient temperature, the disclosed conductive composite materials can be used as temperature sensors that measure changes in ambient temperature according to measured changes in the bulk resistance of the respective conductive composite material.

In order to account for the bulk resistance of the composite material and the parallel resistance paths described herein, the processor of the computer disclosed herein can be programmed to accurately determine the actual change in contact resistance experienced at each sensing point 12 of the sensor 10 based on the digital output signal received from the A/D converter of the data acquisition terminal. In one aspect, the processor can be configured to calculate contact resistance changes at individual sensing points based on the current measurements at each respective sensing point. In this aspect, the processor can calculate the resistance changes as the solution to a series of non-linear equations that describe the load in terms of the current measurements at each respective sensing point. It is contemplated that the processor can be configured to solve the series of simultaneous non-linear equations using one or more conventional algorithms, including, for example and without limitation, the “Newton-Raphson method” and the “node analysis” method. The contact resistance changes calculated by the processor can then be used to determine the actual applied load at each respective sensing point 12.

Thus, in contrast to conventional thin-film load and pressure sensors which calculate loads based on changes in bulk resistance through the depth of the sensor, the contact sensors disclosed herein can calculate loads based on the surface contact characteristics at a junction formed between two electrically conductive members. Specifically, when the electrically conductive members are substantially rigid, a contact sensor 10 as disclosed herein can abut or contact the electrically conductive members such that the contact surface 8 of the contact sensor is in the static position.

In one exemplary aspect, in use, after the contact surface 8 of the contact sensor 10 is positioned in the static position therebetween the electrically conductive members, the contact sensor will measure a contact resistance that varies with the load applied to the contact surface. Because the contact surface 8 of the contact sensor 10 is substantially in a static position, as a conductive member applies a load to the contact surface of the contact sensor, the total surface area of the contact sensor that is in contact with the conductive member will gradually increase as the applied load increases. As this surface contact area increases, the contact resistance across the contact surface 8 of the contact sensor 10 will decrease, thereby increasing the current within the contact sensor (assuming a constant applied voltage). Accordingly, the contact sensors 10 disclosed herein are configured to detect variations in the electrical signal created by contact between one or more conductive members and the electrically conductive composite material of the contact sensors. These variations in the electrical signal correspond to variations in the load applied to the contact sensor 10 by the conductive members.

In yet another aspect, the conductive polymer composite can have a thickness ranging from about 0.001 inches to about 0.100 inches, preferably between about 0.003 inches to about 0.030 inches, resulting in an overall flexible form of the conductive polymer composite. It is contemplated that, although the conductive polymer composite is relatively thin and flexible, the surface of this conductive polymer composite can behave in substantially the same manner as the surface of a substantially rigid conductive polymer composite as described herein. Therefore, it is contemplated that when the thin, flexible conductive polymers disclosed herein are sandwiched between two thin and flexible conductive members, the total surface area of the contact surface of the thin, flexible conductive polymer composite that is in contact with the conductive members can increase as an increasing load is applied to one or more of the thin and flexible sensors. Therefore, it is contemplated that the changes in the surface resistivity of the material forming the thin, flexible sensor 10 can be measured for the thin, flexible polymer composite in the same manner as the substantially rigid conductive polymer composite. By surface, it is meant the surface portions of the sensor 10 that extend to a depth of about 50 nm, more preferably to a depth of about 100 nm, and most preferably to a depth of about 1,000 nm, in the same manner as the contact surfaces of thicker, substantially rigid conductive polymer composites as described herein.

It is contemplated that the conductive paths produced by the plurality of sensing points 12 can vary depending on the spatial arrangement of the sensing points. For example, the conductive paths produced by sensing points 12 in a parallel and evenly spaced configuration can be substantially different than the conductive paths produced when the sensing points are positioned in overlapping, staggered, or unevenly spaced configurations.

In use, and with reference to FIG. 2, after the contact surface 8 of the sensor 10 is positioned in the static position, upon contact of a single sensing point 12 with the electrically conductive joint element, an electrical signal can be generated and sent via wire 18 to a data acquisition terminal as described herein. In one aspect, this electrical signal can be sent in response to a voltage excitation signal that is processed to the electrical signal by the data acquisition terminal. As one skilled in the art will appreciate, in this example, the joint element can act as a first electrode that is mechanically and electrically coupled to the polymeric composite material, which is in turn electrically coupled to a second electrode, i.e., the wire 18. The electrically coupled respective first and second electrodes and the polymeric composite material form an electrical circuit. Though not expressly shown in the Figure, in this particular aspect, each sensing point 12 of the plurality of sensing points can be wired so as to provide data from that point to the data acquisition terminal. It is contemplated that the characteristics of the generated electrical signal can vary with the load applied to the contact surface proximate the sensing point 12, and a dynamic contact stress distribution profile for the joint can thereby be developed.

The surface area and geometry of any individual sensing point 12 as well as the overall geometric arrangement of the plurality of sensing points 12 over the contact surface 8 of the sensor 10, can be predetermined as desired. For example, through the formation and distribution of smaller sensing points 12 with less intervening space between individual sensing points 12, the spatial resolution of the data can be improved. While there may be a theoretical physical limit to the minimum size of a single sensing point determined by the size of a single polymer granule, practically speaking, the minimum size of the individual sensing points will only be limited by modern machining and electrical connection forming techniques. In addition, increased numbers of data points can complicate the correlation and analysis of the data. As such, the preferred geometry and size of the multiple sensing points can generally involve a compromise between the spatial resolution obtained and complication of formation methods.

In this particular aspect as seen in FIG. 2, the composite material forming the surface sensing points 12 can extend to the base 15 of the sensor 10, where electrical communication can be established to a data acquisition and analysis module, such as a computer with suitable software, for example.

In one aspect, the discrete sensing points 12 of the sensor 10 of FIG. 2 can be separated by a non-conductive material 14 that can, in one aspect, be formed of the same polymeric material as that contained in the composite material forming the sensing points 12. In general, the method of combining the two materials to form the sensor can be any suitable formation method. For example, in one aspect the composite material can be combined with a virgin material to produce one or more sensor sheets as described herein. Alternatively, the composite material can be formed into a desired shape, such as multiple individual rods of composite material as shown in the aspect illustrated in FIG. 2, and then these discrete sections can be inserted into a block of the non-conductive polymer that has had properly sized holes cut out of the block. Optionally, the two polymeric components of the sensor can then be fused, such as with heat and/or pressure, and any final shaping of the two-component sensor, such as surface shaping via machining, for example, can be carried out so as to form the sensor 10 including discrete sensing points 12 formed of the conductive composite material at the surface 8.

In many aspects of the invention, the same material, but for the presence or absence of the conductive filler, can be used for the composite sensing points 12 and the intervening spaces 14 since, as described above, the physical characteristics of the composite material can be essentially identical to the physical characteristics of the non-conductive material used in forming the composite. According to this aspect, the sensor 10 can have uniform physical characteristics across the entire sensor 10, i.e., both at the sensing points 12 and in the intervening space 14 between the sensing points.

In one particular aspect, the polymer used to form the sensor 10 can be the same polymer as is used to form the member for use in the field. For example, when considering the examination of artificial joints, the polymer used to form both the composite material at the sensing points 12 and the material in the intervening space 14 between the sensing points 12 can be formed of the same polymer as that expected to be used to form a polymeric bearing component of an implantable device (e.g., UHMWPE or PPS). Thus, the sensor 10 can provide real-time, accurate, dynamic contact data for the implantable polymeric bearing under expected conditions of use.

Optionally, the surface 8 of the sensor 10 can be coated with a lubricating fluid, and in particular, a lubricating fluid such as can be utilized for the bearing component of the implantable device during actual use and under the expected conditions of use (e.g., pressure, temperature, etc.). In this aspect, in addition to providing direct contact data, the disclosed sensors can also be utilized to examine data concerning contact through an intervening material, i.e., lubrication regimes under expected conditions of use. For example, the sensor can be utilized to determine the type and/or quality of lubrication occurring over the contact surface of the sensor, including variation in fluid film thickness across the surface during use. In one aspect, this can merely be determined by presence or absence of fluid, e.g., presence or absence of direct contact data (i.e., current flow) in those aspects wherein the fluid is a non-conductive lubricating fluid. In other aspects, a more detailed analysis can be obtained, such as determination of variation in fluid film thickness. This information can be obtained, for example, by comparing non-lubricated contact data with the data obtained from the same joint under the same loading conditions but including the intervening lubricant. In another aspect, such information could be obtained through analysis of the signal obtained upon variation of the frequency and amplitude of the applied voltage. In yet another aspect, the sensor can be utilized in a capacitance mode, in order to obtain the exact distance between the two surfaces forming the joint. In one particular aspect, the disclosed sensor can be utilized to determine a lubrication distribution profile of the contact surface over time.

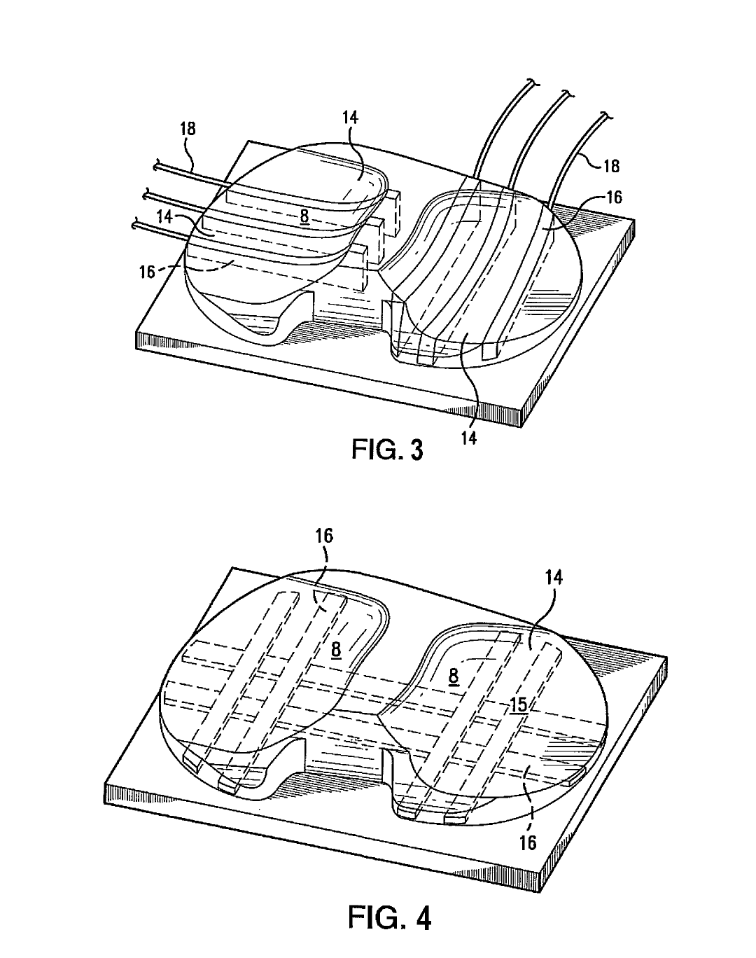

FIG. 3 illustrates another aspect of the contact sensors as described herein. According to this aspect, the sensor 10 includes multiple sensing strips 16 across the contact surface 8 of the sensor. As illustrated, in this aspect, the orientations of the individual sensing strips 16 across the different condoyles formed on the contact surface can be selectively varied. Alternatively, and as shown in FIG. 4, strips can be laid in different orientations on separate but identically shaped sensors in a multi-sensor testing apparatus. In any case, by varying the orientation of sensor strips on multiple, but essentially identical surfaces, virtual cross-points can be created when the data from the different surfaces is correlated. In particular, when contacts of the same shape and magnitude at the same location of different surfaces are recognized, a virtual data point at the cross-point can be created. As can be seen in FIG. 4, this aspect can allow the formation of fewer electrical connections and wires 18 in order to provide data to the acquisition and analysis location, which may be preferred in some aspects due to increased system simplicity.

Optionally, it is contemplated that the contact sensors as described herein can be utilized to provide sub-surface stress data. For example, in the aspect illustrated in FIG. 3, multiple sensing strips 16 can be located within a subsurface layer at a predetermined depth of the sensor. According to this aspect, the horizontal and vertical strips 16 can cross each other with a conductive material located between the cross points to form a subsurface sensing point 15 at each cross point. In one aspect, the strips 16 can be formed of the composite material described herein with the intervening material being the same basic composite material but with a lower weight percentage of the conductive filler, and the layer can be laid within the insulating non-conductive polymer material 14. In another aspect, the sensing strips 16 can be any conductive material, such as a metallic wire, for example, laid on either side of a sheet or section of the composite material and the layer can then be located at a depth from the surface 8 of the sensor.

Application of a load at the surface 8 of the sensor can then vary the electronic characteristics at the internal sensing point 15. In particular, the current flow at any sensing point 15 can vary in proportion to the stress at that sensing point. Thus, when data from multiple sensing points 15 are correlated, an internal stress profile for the sensor can be developed at the depth of the sensing points.

In yet another aspect, in lieu of strips, the conductive filler may be arranged on the sensor sheet as a plurality of dots, as shown in FIG. 5. In this aspect, there would be reduced opportunity for cross-talk when the sensor sheets were thermoformed into shape. In this aspect, it can be appreciated that the electrical connections necessary to perform the load analysis can be challenging due to the number of connections required. As such, application of current to one of the sheets may be achieved using a sheet of flexible conductive material, such as, for example and without limitation, mesh, foil, and the like. In use, a sensor sheet having a plurality of conductive dots can be configured for coupling with electrodes proximate each respective conductive dot. Following application of a load with a metallic or other conductive element, it is contemplated that current can flow through the conductive filler therein the sensor sheet, thereby permitting calculation of the applied loads.

FIG. 6 is a schematic of a contact sensor in operative communication with a data acquisition terminal. As depicted in FIG. 6, a battery can be operatively coupled to the data acquisition terminal, and a computer can be coupled to the data acquisition terminal via a Wi-Fi transmitter.

In use, it is contemplated that a plurality of sensor sheets can be thermoformed in substantially identical three-dimensional sizes and orientations. In one aspect, the sensor sheets can be placed in a stacked relationship with adjacent sensor sheets. In this aspect, it is contemplated that no fusing between adjacent sensor sheets will occur. In another aspect, the configurations of the portions of conductive filler therein the sensor sheets can be selected to create overlap between the conductive portions of adjacent sensor sheets. For example, and without limitation, the conductive portions of one sensor sheet can be oriented substantially perpendicularly to the conductive portions of an adjacent sensor sheet prior to stacking of the sensor sheets. It is contemplated that upon application of a load to the sensor sheets, each respective sensor sheet can function as an electrode such that no additional contact with a conductive element is required to produce current therethrough the sensors sheets. It is further contemplated that the overlap between the conductive portions of the sensor sheets can create cross points for measuring loads applied to the sensor sheets.

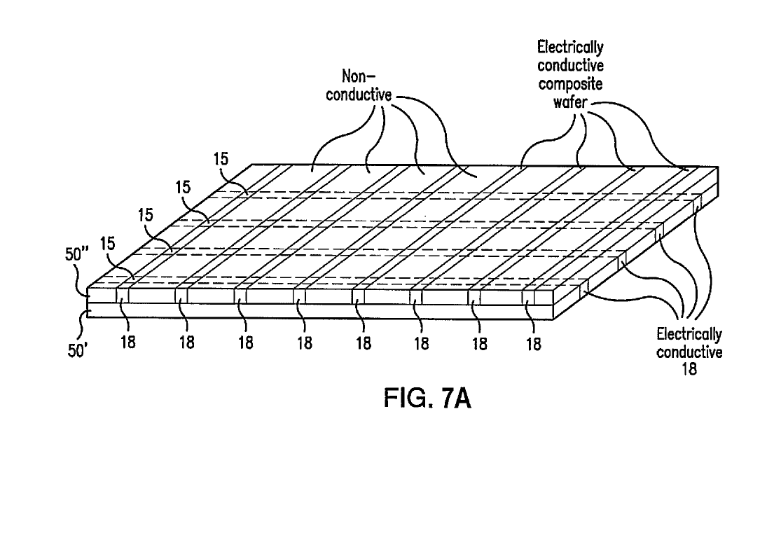

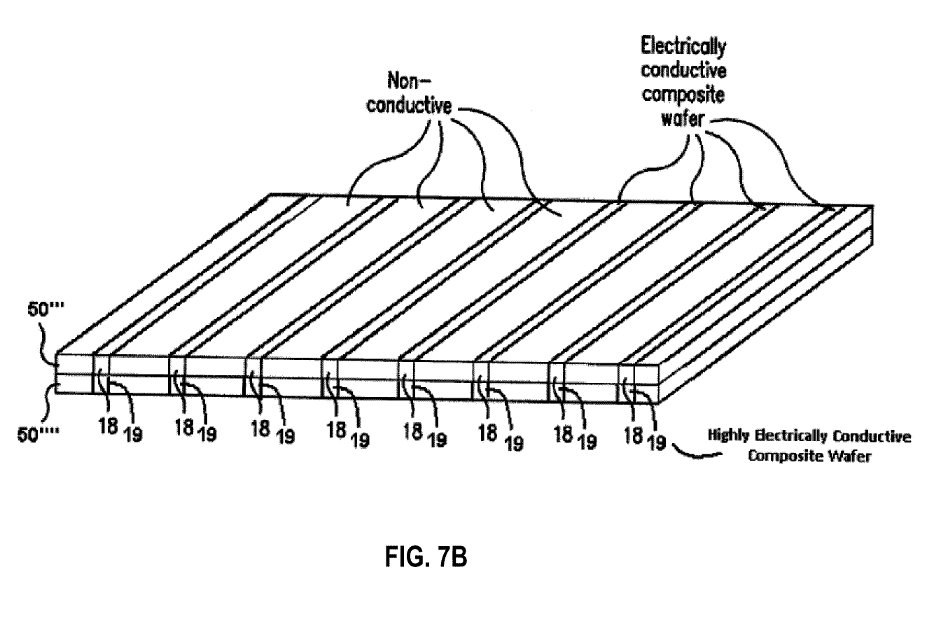

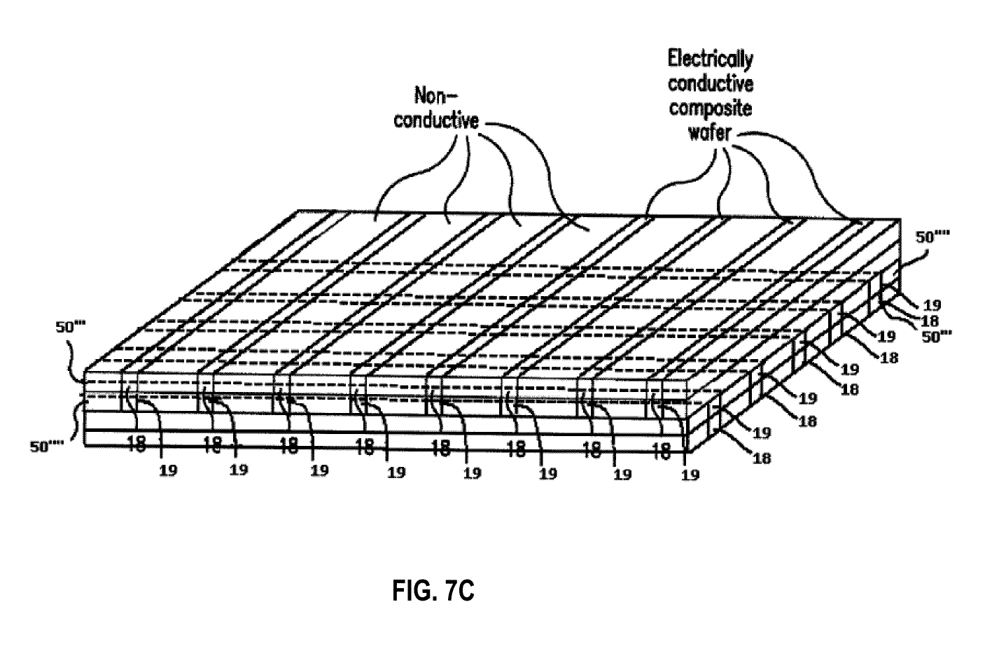

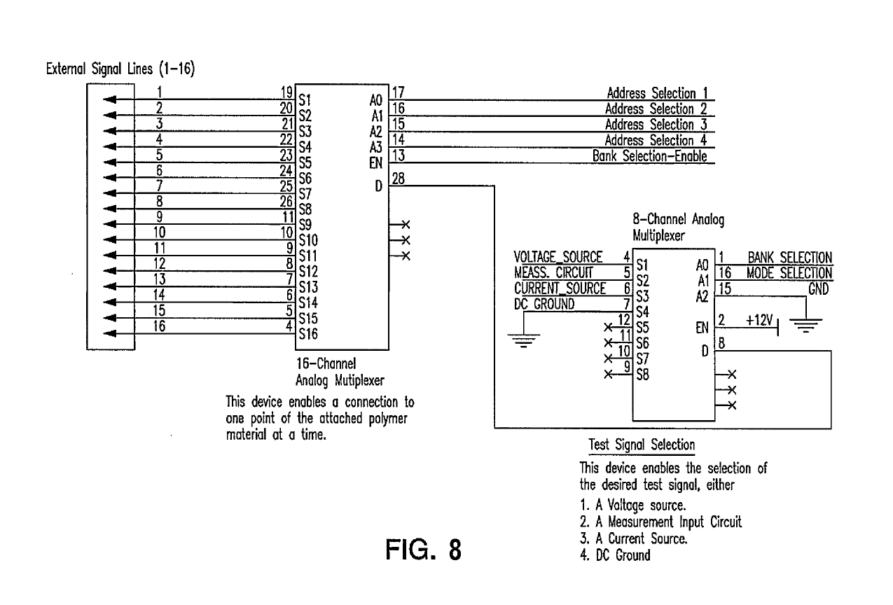

In another aspect, as shown in FIG. 7A, the sensors can include multiple stacked polymer sensor sheets. In one exemplary aspect, each polymer sensor sheet can have a plurality of conductive stripes of conductive material that are separated by non-conductive polymeric stripes. In the illustrated example, the vertical conductive stripes on one sheet, “columns,” and the horizontal conductive stripes on the underlying sheet, “rows,” are positioned relative to each other so that, at the places where these columns and rows spatially intersect, the conductive areas of the two sheets are in physical and electrical contact with each other. In one aspect, the exemplary interface electronics illustrated in FIG. 8 can be used with appropriate control software within the data acquisition terminal to connect one column to a voltage source and one row to a current-to-voltage circuit, in order to measure the current through the conductive polymer materials. In one aspect, it is contemplated that each column/row pair, i.e., the internal junction points 15, can be measured, one at a time, to provide a complete set of current measurements. As illustrated, the substantially perpendicular relative orientation of the stacked sensor sheets can allow for formation of an array of sensing points by the overlapping portions of the conductive stripes of the stacked sensor sheets.

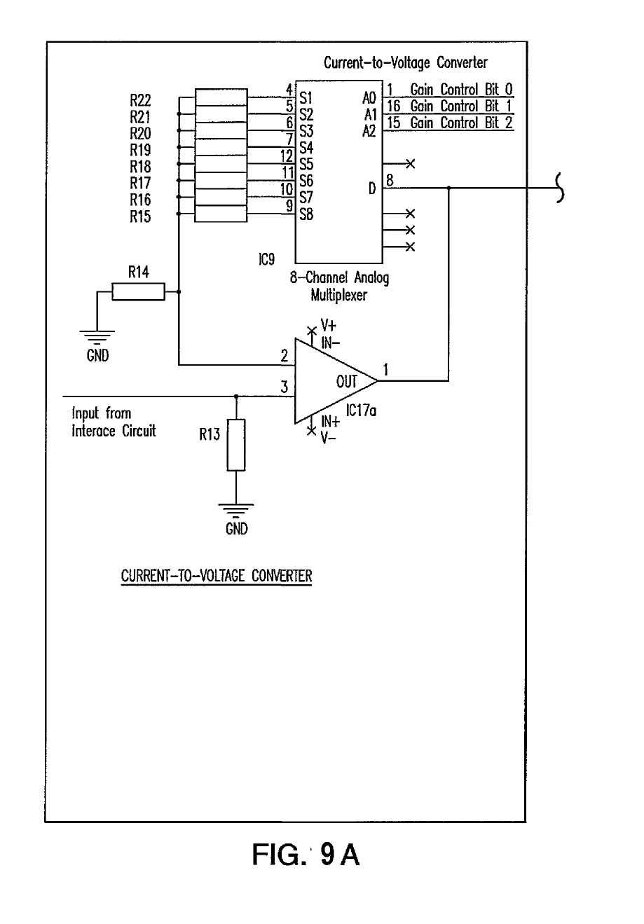

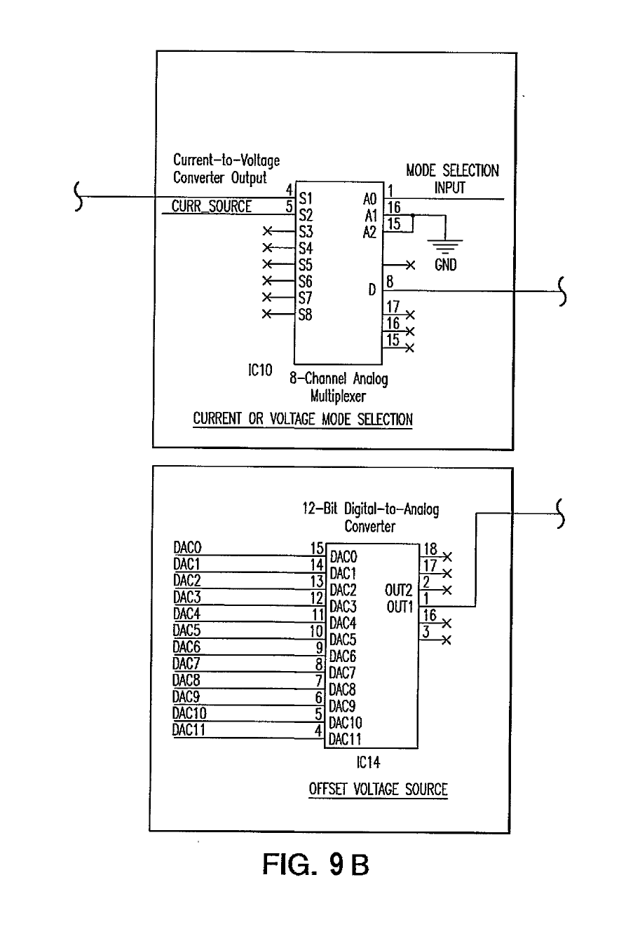

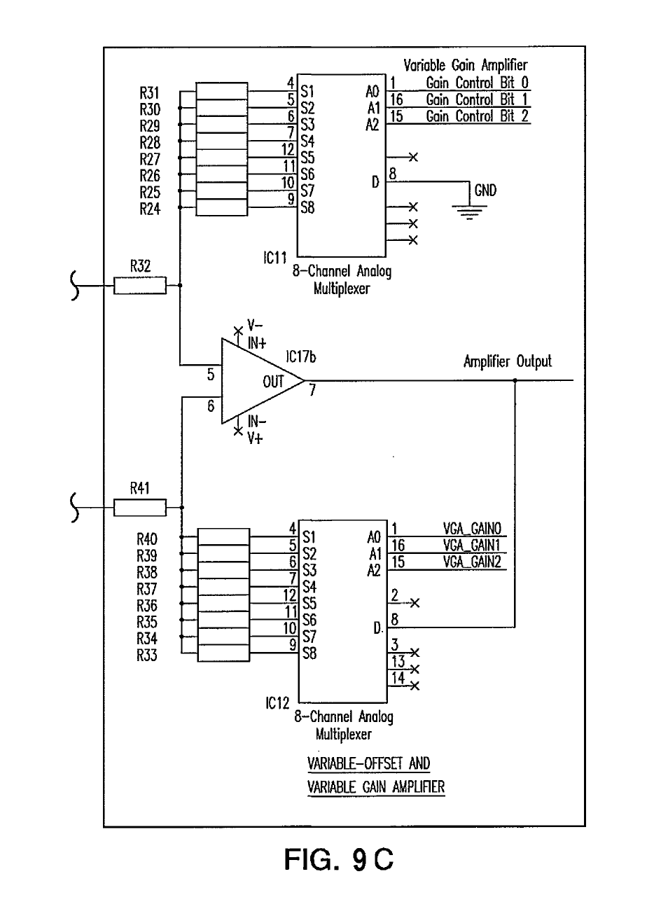

In one aspect, these current measurements do not represent the currents at the pressure-sensitive points in the stacked polymer sheets where the stripes overlap. Rather, the current measurements can be external measurements at external points (also called “nodes”), which are generally near the outer edges of the material. The measurement data are processed in software within the data acquisition terminal in order to calculate the individual currents that are present at each measurement point where the columns and rows overlap, and then this information is used to determine the pressure that is applied at each measurement point. An exemplary, non-limiting, schematic of the measurement circuitry is provided in FIGS. 9A-C herein.

In yet another aspect, both subsurface contact data and surface contact data can be gathered from a single sensor through combination of the above-described aspects.

As described below with respect to the embodiment shown in FIGS. 7B and 7C, there is no need for use of the node analysis method outlined above as the respective nodes act as if there are directly “wired.”

FIG. 7B illustrates another exemplary aspect of a composite sensor sheet for use in a sensor disclosed herein for obtaining pressure data of a junction. In this aspect, each composite sensor sheet has two stacked, adjoined sheets 50′″, 50″″. In one aspect, each stacked sheet 50′″, 50″″ can have a plurality of conductive stripes 18, 19 of conductive polymeric material that are separated by non-conductive polymeric stripes. However, in this aspect, the plurality of conductive stripes 18 on sheet 50′″ is more conductive than the plurality of conductive stripes 19 on the adjoining sheet 50″″. As shown, in one aspect, the respective stacked adjoining sheets 50′″, 50″″ of the composite sensor sheet are oriented substantially parallel to and overlying each other. As one will appreciate, the absolute conductivity levels between the plurality of conductive stripes 18, 19 in the respective stacked, adjoined sheets 50′″, 50″″ can also vary, with a requirement that there been an effective variance in conductivity levels between the adjoining plurality of conductive stripes 18, 19 in the respective stacked, adjoined sheets 50′″, 50″″.

Referring now to FIG. 7C, in another aspect of the sensor disclosed herein for obtaining pressure data of a junction, two stacked composite sensor sheets as shown in FIG. 7B are adjoined together so that the respective stacked composite sensor sheets are oriented substantially perpendicular to each other. Thus, an array of sensing points is formed by the overlapping portions of the conductive stripes of the two stacked composite sensor sheets. As shown, and not meant to be limiting, one example of the resulting formed composite structure of the sensor can have four adjoining sheets, forming four layers of conductive material that vary, layer to layer, from a higher conductivity (top sheet 50′″), to a second and third lower conductivity (middle sheets 50″″), back to the higher conductivity (bottom sheet 50′″). In this exemplary embodiment, the respective outside layers 50′″ effectively operate as wires.

It is contemplated that the respective stacked adjoining sheets 50′″, 50″″ of the composite sensor sheets can be substantially the same thickness, or the respective stacked adjoining sheets 50′″, 50″″ can vary in respective thickness. In this embodiment, it should be appreciated that there is no requirement that sheet 50′ containing the plurality of conductive stripes 18(which operative act as “wires” for the sensor) be of any given thickness or, optionally, of the same thickness. In one exemplary aspect, it may be advantageous in thermoforming applications for sheet 50′″ to be significantly thicker than the thinner sensor sheet prior to lamination.

In this aspect, it is contemplated that the composite sensor sheets can be formed from a wide range of conventional polymers and conductive fillers. It is also contemplated that the polymeric composition of the respective sheets can be the same or can vary between the respective stacked adjoining sheets 50′″, 50″″.

In one aspect, the plurality of conductive stripes 19 on sheet 50″″ can have about a 1-10% conductive carbon black loading. Optionally, the plurality of conductive stripes 18 on sheet 50′″ can have about a 10-30% conductive carbon black loading.

In exemplary aspects, the polymer sheets 50′″, 50″″ can each comprise a sheet of non-conductive HDPE that acts as a carrier for the plurality of conductive stripes 18, 19. In these aspects, the plurality of conductive stripes 18, 19 can comprise HDPE and a desired weight percentage of carbon black. It is contemplated that the conductive stripes can be formed by overlying an HDPE stripe with a carbon black stripe of corresponding size. It is further contemplated, without limitation, that the carbon black stripes can have a thickness ranging from about from about 0.001 inches to about 0.100 inches, preferably between 0.003 inches to about 0.010 inches It is still further contemplated that, in an overlying configuration, the corresponding HDPE stripes and carbon black stripes can be laminated to the sheet of non-conductive HDPE to form the polymer sheets 50′″, 50″″. It another aspect, the sheet of non-conductive HDPE can have a different color than the conductive HDPE stripes 18, 19 such that, when the conductive HDPE stripes are laminated to the sheet of non-conductive HDPE, a series of stripes of alternating colors is formed. In one example, and without limitation, the sheet of non-conductive HDPE can be colored white, while the conductive HDPE stripes 18, 19 can be colored black.

In some aspects, and as shown in FIGS. 7A-7C, it is contemplated that polymer sheets 50′″, 50″″ can be laminated to one another such that conductive stripes 18 overlie conductive stripes 19. In these aspects, the laminated structure formed from polymer sheets 50′″, 50″″ can be cut to any desired dimensions. For example and without limitation, the formed laminated structure can have a substantially square shape, with a length of about 14 inches and a width of about 14 inches. In use, it is contemplated that a first of such laminated structures can be positioned in overlying relation to a second laminated structure to form a sensor array. In one exemplary aspect, it is contemplated that the two laminated structures can be positioned such that the conductive stripes of the respective polymer sheets of the first laminated structure are substantially perpendicular to the conductive stripes of the respective polymer sheets of the second laminated structure. In this aspect, it is contemplated that each laminated structure can comprise 12 conductive stripes such that, when the laminated structures are positioned in overlying relation, a sensor array of 144 sensing points is formed.

In one non-limiting exemplary aspect, the polymer sheet 50′″ can comprise a layer of non-conductive HDPE that is laminated to a plurality of conductive stripes 18, with each conductive stripe comprising a stripe of carbon black (in a weight percentage ranging from between about 0.5% to about 30%, and preferably between about 1% to about 10%) that overlies a stripe of HDPE, which can be of a corresponding size. In this aspect, it is contemplated that the plurality of conductive stripes 18 can be spaced apart from adjacent conductive stripes by between about 0.01 inches to about 0.20 inches, or preferably, about 0.06 inches. It is further contemplated that polymer sheet 50′″ can function as a high contact resistance signal carrier.

In an additional non-limiting exemplary aspect, the polymer sheet 50″″ can comprise a layer of non-conductive HDPE that is laminated to a plurality of conductive stripes 19, with each conductive stripe comprising a stripe of carbon black in a weight percentage of between about 0.5% to about 30%, and preferably about 25% that overlies a stripe of HDPE, which can be of a corresponding size. In use, it is contemplated that polymer sheet 50″″ can function as a low contact resistance signal carrier. Of course, although the above sensor sheets are described with respect to HDPE and carbon black, it is contemplated that other materials, such as those described herein, can be used to practice the invention.

In use, it is contemplated that the disclosed sheet-like sensors can be applied in a variety of positions and orientations. In one aspect, the sensors can be applied in a flat configuration. In another aspect, the sensors can be applied when the sensors are flexed along an axis. When the sensors are used in flat or flexed configurations, it is contemplated that the sensors can be pre-calibrated to be flat using a foam interface to ensure even pressure distribution. In an additional aspect, the sensors can be thermoformed to have a desired three-dimensional shape and orientation. In this aspect, it is contemplated that the sensors can be calibrated using a balloon and fabric rig with a conventional pressure meter.

In alternative aspects, its is contemplated that two different types of conductive fillers can be used, for example and without limitation, carbon black material having differing conductivity can be used in the respective conductive stripes 18, 19. For example, a normal carbon black, and a highly conductive carbon black can be used. In this exemplary aspect, the normal carbon black can be loaded into the polymer in a range between about 0.1% to 10% (by weight) to form the conductive stripes 19 on sheet 50″″, which forms a conductive polymer with a high surface resistivity. Further, the low bulk resistivity conductive stripes 18 on sheet 50′″ can be formed by mixing between about 10% to 30% of the highly conductive carbon black into the polymer. Thus, in this optional embodiment, the lower bulk resistivity conductive polymer stripe 18 on sheet 50′″ can use both a greater carbon black loading and a more conductive carbon black.

The sheet 50′″, having a plurality of conductive stripes 18 of higher conductivity with respect to the adjoining plurality of conductive stripes 19 therein sheet 50″″, has significantly less volume or bulk resistance. As one skilled in the art will appreciate, the plurality of stripes 19 on the adjoining middle sheets 50″″ are less conductive (more resistance), and the plurality of stripes 19 on the outside sheets 50′″ are more conductive (the outside ones act as the “wires”). Therefore, it is contemplated that the supplied electrical current seeks or takes the path of least resistance and flows to ground through the rows of higher conductivity provided in sheet 50′″.

As one will appreciate, the formed sensor described above with respect to FIGS. 7B and 7C has layers of conductive stripes 19 that exhibit very low bulk resistivity and a very high surface resistivity. Conventional materials typically exhibit a bulk resistivity that is proportional to the contact (or surface) resistivity. For example, both bulk and contact resistivity in conventional materials will, be low, high, or in between, such that when you change either the bulk or contact resistivity, the same change is effected in the other resistivity. However, in one exemplified embodiment, the formed sensor has layers of conductive stripes 19 having a low bulk resistivity and a high surface resistivity, such that the bulk resistivity is substantially zero when compared to the magnitude of the surface resistivity. The low volume or bulk resistivity of the layers of conductive stripes 19 of the sensor effectively acts as a conventional “wire.”

In one aspect, the orders of magnitude for the bulk resistivity of the formed sensor illustrated in FIGS. 7B and 7C can be on the order of 10̂2 ohm-in and the order of magnitude of the surface resistivity can be on the order of 10̂5 to 10̂10 ohms/sq. It is contemplated that the greater the difference the respective bulk and surface resistivity, the greater the exemplified sensor will perform.

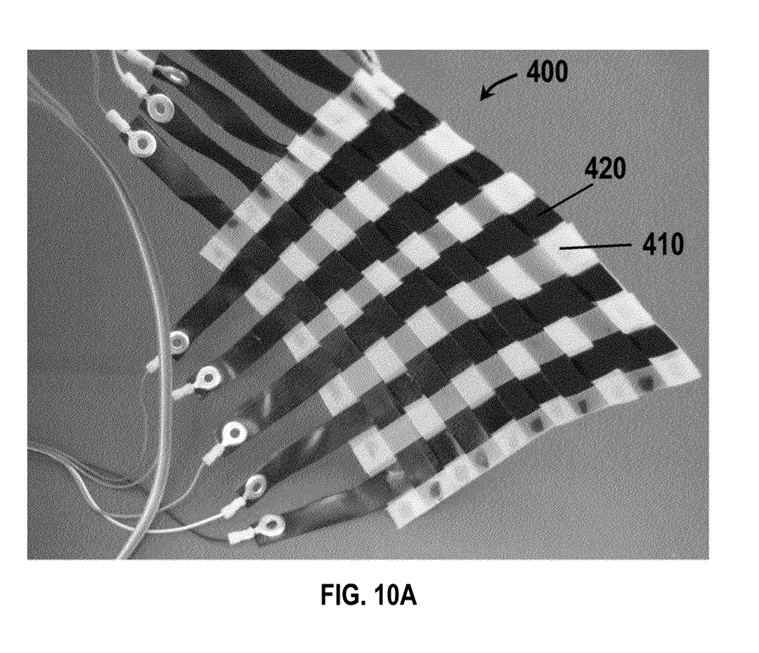

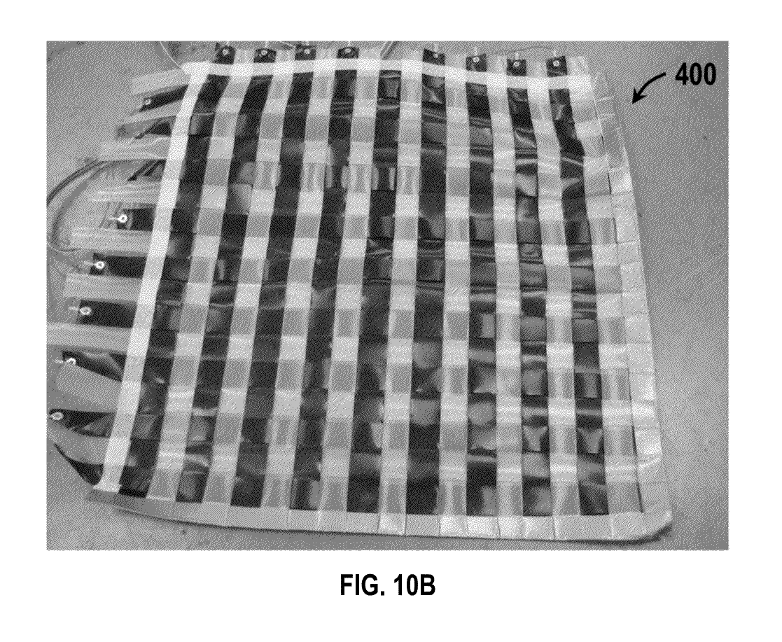

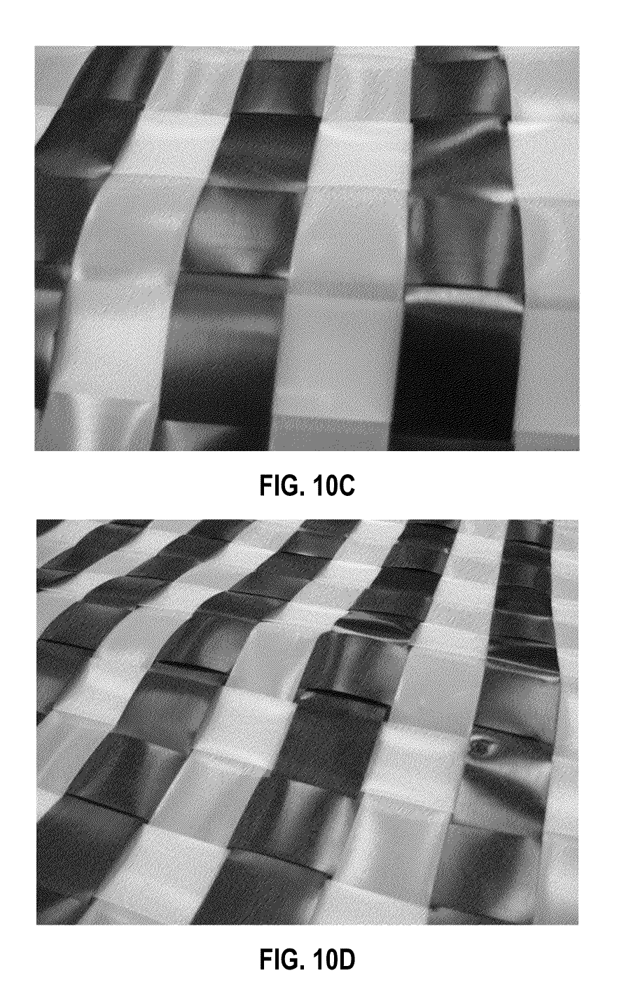

In additional aspects, as shown in FIGS. 10A-10D, the sensor sheets 400 can comprise a series of spaced non-conductive HDPE stripes 410 that are interwoven with a series of spaced conductive stripes 420 comprising HDPE and a desired weight percentage of carbon black. In these aspects, it is contemplated that the non-conductive HDPE stripes 410 can have a different color than the conductive HDPE stripes 420 such that, when the conductive HDPE stripes are interwoven with the non-conductive HDPE stripes, a checkerboard pattern of alternating colors is formed. In some aspects, the non-conductive HDPE stripes 410 can be colored white, while the conductive HDPE stripes 420 can be colored black. Although the above sensor sheets 400 are described with respect to HDPE and carbon black, it is contemplated that other materials, such as those described herein, can be used to practice the invention.

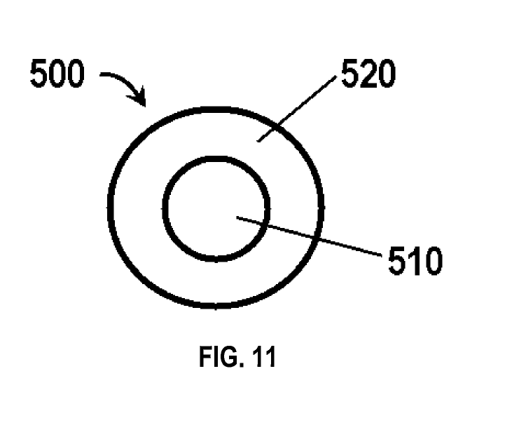

In a further aspect, as shown in FIG. 11, the sensor sheets can comprise a plurality of interwoven sensor filaments 500. In this aspect, the sensor filaments 500 can comprise a copper wire core 510 onto which a conductive sensor material 520, such as, for example and without limitation, UHMWPE with a desired weight percentage of carbon black, is extruded. It is contemplated that the sensor filaments 500 can be woven together in an overlapping and intersecting pattern such that a sensor sheet is formed. In exemplary aspects, the sensor filaments 500 can have a gauge ranging from about 20 to about 40, including, for example and without limitation, 20 gauge, 21 gauge, 22, gauge, 23, gauge, 24 gauge, 25 gauge, 26 gauge, 27 gauge, 28 gauge, 29 gauge, 30 gauge, 31 gauge, 32 gauge, 33 gauge, 34 gauge, 35 gauge, 36 gauge, 37 gauge, 38 gauge, 39 gauge, and 40 gauge.

In one aspect, the sensor may comprise a thermoformable polymer, such as, for example and without limitation, ultra high molecular weight polyethylene (UHMWPE), high density polyethylene (HDPE), polyphenylene sulfide (PPS), low density polyethylene (LDPE), acrylonitrile butadiene styrene (ABS), polyvinyl chloride (PVC), nylon, or polyoxymethylene copolymer (POM).

In one aspect, the sensor can be formed into any desired shape. For example, the sensor can be formed into the shape of at least a portion of an artificial joint bearing, such as, for example and without limitation, a portion of an artificial joint, a portion of a prosthetic limb, or other prosthesis. Pressure mapping of portions of a joint bearing can provide data necessary to fit the prosthesis to the user with lower wear. In this aspect, a polymer capable of stretching is advantageous due to the non-uniformity of the shape of the prosthesis.

In an additional aspect, it is contemplated that the contact sensors can have a desired hardness. Specifically, when the sensor comprises PPS, it is contemplated that the desired hardness can be about M93 (R125) on the Rockwell scales and about D 85 on the Shore D scale. When the sensor comprises UHMWPE, it is contemplated that the desired hardness can be about D 61 on the Shore D scale. When the sensor comprises ABS, it is contemplated that the desired hardness can be about R105 on the Rockwell scale. When the sensor comprises nylon, it is contemplated that the desired hardness can be about R120 on the Rockwell scale. When the sensor comprises PVC, it is contemplated that the desired hardness can be about R112 on the Rockwell scale. When the sensor comprises POM, it is contemplated that the desired hardness can be about R120 on the Rockwell scale.

In another aspect, the sensors can be manufactured in a two stage process. First, the non-conductive sheets of thermoformable polymer can be molded from raw material. Second, the conductive strips can be added to the sensor sheet and placed back into the same mold. In this manner, flow of the non-conductive polymer into the conductive region of the sheet, and flow of the polymer with conductive filler into the non-conductive region of the sheet can be minimized to ensure that, when thermoformed, there is no crosstalk between adjacent conductive strips.

In this aspect, calibration of the each sensor can be performed prior to the thermoforming step, as calibration after thermoforming can prove to be more difficult. It is believed that the characteristics of the sensors do not substantially change during the thermoforming process.

In one aspect, such calibration may be desired as each individual sensor can have individually unique electrical properties that must be calibrated to a standard in order to achieve a desired degree of load measurement accuracy. Further, it is believed that the individual sensors can experience hysteresis when the sensors are unloaded. Thus, it is contemplated that conventional signal processing components configured to correlate the voltage or current to the load of the respective sensor can be implemented using software configured to correlate the load during loading and load during unloading. To compensate for the observed hysteresis effect, it is also contemplated that the software can be configured to calculate the load during a static position—when the load is substantially constant—by using a mean point between a calculated load value during loading and a calculated load value during unloading.

When used in making a prosthetic limb, for example, the sensor sheets can be thermoformed into the shape of a cup for receiving the anatomical limb. Once the sheets are used to map out the force distribution in the cup, the sensor sheets can be adjusted accordingly. This process can be repeated until the forces are substantially uniformly distributed as desired. Once the desired level of force distribution is achieved, a mold, such as for example, a plaster mold, can be made of the interior portion of the cup. Then the mold can be used to form the cup out of materials that are suitable for the prosthesis.

Optionally, the composite materials produced as described herein can be incorporated into one or more sensor sheets. In one aspect, a method for producing the sensor sheets can comprise providing a plurality of substantially circular virgin sheets comprising at least one virgin material. In this aspect, the virgin material can comprise, for example and without limitation, virgin UHMWPE. In another aspect, the method for producing the sensor sheets can comprise providing a plurality of substantially circular composite sheets comprising at least one composite material as disclosed herein. In this aspect, the composite material can comprise, for example and without limitation, a mixture of carbon black and UHMWPE. In an additional aspect, the virgin sheets can have an outer diameter substantially equal to an outer diameter of the composite sheets. In yet an another aspect, the virgin sheets can have an inner diameter substantially equal to an inner diameter of the composite sheets. In a further aspect, the method for producing the sensor sheets can comprise positioning the virgin sheets and the composite sheets can be stacked in a desired configuration. In this aspect, the desired configuration can comprise a single stack of alternating virgin and composite sheets such that virgin sheets are intermediate and in contact with composite sheets and composite sheets are intermediate and in contact with virgin sheets.

In one exemplary aspect, while the virgin and composite sheets are stacked in the desired configuration, the virgin and composite sheets can be subjected to a conventional compression molding process for heating and then fusing the virgin and composite sheets together. In this aspect, the virgin material can comprise UHMWPE. In another aspect, the compression molding of the virgin and composite sheets can produce a substantially cylindrical billet. In this aspect, the substantially cylindrical billet can be substantially hollow. In a further aspect, the billet can be placed on a conventional mandrel. In this aspect, the mandrel can be configured to spin at a desired rate. In still a further aspect, the method for producing the sensor sheets can comprise spinning the mandrel, thereby turning the billet as the mandrel spins. In another aspect, the method can comprise subjecting the billet to a conventional skiving machine. It is contemplated that the skiving machine can comprise a blade for slicing or shaving off a thin layer of the billet. In operation, the blade of the skiving machine advances toward the billet at a constant rate as the billet rotates on the mandrel, thereby producing the sensor sheets. In one aspect, the sensor sheets can be of substantially uniform thickness. In this aspect, it is contemplated that the sensor sheets can have a thickness ranging from about 0.001 inches to about 0.050 inches, more preferably from about 0.002 inches to about 0.030 inches, and most preferably from about 0.003 inches to about 0.020 inches.

In another exemplary aspect, while the virgin and composite sheets are stacked in the desired configuration, the virgin and composite sheets can be subjected to a conventional compression molding process for separately heating and shaping the virgin and composite sheets. In this aspect, the virgin material can comprise PPS. In an additional aspect, the virgin and composite sheets can be joined together using a glue, such as, for example and without limitation, a cyanoacrylate, an epoxy, and the like. As one skilled in the art will appreciate, because PPS and many other conventional polymers have a significantly lower melt viscosity that UHMWPE, compression molding of the composite material with the PPS would lead to undesired mixing of the virgin and composite materials, thereby destroying the existence of discrete conductive and non-conductive portions on the contact surface.

In a further aspect, prior to joining of the virgin and composite materials, it is contemplated that the surfaces of the virgin and composite sheets can be subjected to one or more desired treatments. In this aspect, the one or more desired treatments can comprise, for example and without limitation, flame treatment, chemical etching, chemical preparation, and the like. It is contemplated that after gluing of the virgin and composite materials, the virgin and composite materials can form a single, unified element that can be machined without any risk of the individual pieces of material becoming separated from the unified element. Accordingly, it is further contemplated that the resulting element can be selectively machined without producing any gaps or inconsistencies at the junctions between the virgin and composite materials and between multiple sheets of material.

As one of skill in the art will appreciate, the characteristics of the virgin material used to produce the contact sensor can be analyzed to determine the suitability of the contact sensor for particular applications. For example, PPS can be easily sterilized by autoclave sterilization, whereas UHMWPE lacks the temperature resistance needed for autoclaving. Thus, it is contemplated that PPS can be selected as a virgin material for use in contact sensors that need to be re-useable. However, UHMWPE is significantly cheaper than PPS. Therefore, UHMWPE can be selected as a virgin material for use in contact sensors that will be disposable. Similar characteristics, including mechanical and sensitivity properties, of other conventional engineering polymers can also be examined to determine the adequacy of these polymers for use in the contact sensors disclosed herein. One of the above-discussed methods for producing the contact sensors can be selected for each polymer depending on an analysis of the melt viscosity and other characteristics of the polymer.

In another embodiment, and with reference to FIG. 2, it is contemplated that the sensor 10 can be used intraoperatively during orthopedic implant surgery. The sensor 10 can allow for monitoring of, for example and without limitation, at least one of: the i) force between an orthopedic implant or other medical devices and the patient, ii) force or pressure between a trial joint component and the underlying bone, iii) forces internal to a medical device, iv) force or pressure between a trial component and other orthopedic components, v) forces or pressures of surrounding soft tissue structures on the trial component. For example and without limitation, the sensor 10 described herein can be used in association with: a) the tibial, femoral, or patellar components of a prosthesis used in a total knee replacement procedure; b) the femoral or acetabular components of a prosthesis used in total hip implant procedure; c) the scapular or humeral components of a prosthesis in a shoulder replacement procedure; d) the tibia and talus components of a prosthesis used in an ankle replacement procedure; and e) devices implanted between the vertebral bodies in lumbar or cervical spine disk replacement procedures. As one skilled in the art will appreciate, the intra-operative observation of the forces in the joint allows surgeons to better understand the kinematics of the joint, including the effects of load magnitude and/or load imbalance, thereby enabling the surgeon to make critical adjustments regarding component selection, component position, and the performance of intra-operative soft tissue procedures.

For example, in one aspect, the disclosed sensors 10 can use similar materials as those found in an artificial knee implant. It is of course contemplated that the insert can include any polymeric insert portion of any desired implant. However, in this example and for clarity, the description below will describe the insert as a tibial insert that is conventionally sized for use with a knee implant during a TKA procedure. In this aspect, it is contemplated that the tibial inserts of the knee implant can be formed with at least one discrete sensing points 12. The discrete sensing points can be randomly spaced on the contact surface 8 of the sensor 10. Alternatively, the discrete sensing point(s) can be positioned in a predetermined array on the contact surface. In various optional aspects and without limitation, it is contemplated that the discrete sensing point(s) can comprise at least 20% of the surface area of the contact surface of the insert, at least 30% of the surface area of the contact surface of the insert, at least 40% of the surface area of the contact surface of the insert, at least 50% of the surface area of the contact surface of the insert, at least 60% of the surface area of the contact surface of the insert, at least 70% of the surface area of the contact surface of the insert, at least 80% of the surface area of the contact surface of the insert, or at least 90% of the surface area of the contact surface of the insert.

In one aspect, it is contemplated that the tibial insert can be implanted with the knee implant, which provides for operative sensing during and after the implantation procedure, or, optionally, it is contemplated that the tibial insert can be a trial insert. In this latter instance, the trial tibial insert can be inserted so that the soft tissue balancing can be accomplished with active force/pressure feedback on the joint. After the balancing is complete, an implantable tibial insert, of the same dimensions of the trial tibial insert, can replace the trail tibial insert within the implant. In this aspect, the trial tibial inserts can use the sensing technology described herein to quantify the force being applied to each side of the implant, thereby allowing surgeons to more precisely carry out the important step of soft tissue balancing, which, in turn, reduces the rate of early failure of artificial knee joints.

In a conventional TKA procedure, the surgeon typically removes the worn, exposed bone areas on the femur and/or tibia, reshapes the remaining bones, and replaces these damaged bone areas with new, durable artificial implant devices prosthesis. In the procedure, the femur, tibia, and patella are reshaped and prepared to receive the new knee implant prosthesis using conventional surgical alignment tools.

Subsequently, a femoral implant is then attached to the formed reshaped surface on the femur. Next, a tibial tray implant with a polymeric tibial insert is attached to the formed reshaped surface of the tibia. In addition, a patellar implant is coupled to the reshaped surface of the patella. When positioned within the knee, the femoral implant faces and abuts the polymeric tibial insert positioned therein the tibial tray implant. The femoral implant and the tibial tray implant generally have mounting members that extend outwardly from their respective bottom surface that are configured to extend inwardly into the respective femur and tibia bone, which aid in stabilizing and fixing the femoral and metal tray implants with respect to the reshaped bones. Conventionally, the femoral and tibial tray implants are formed of metal material. As one will appreciate, the polymeric tibial insert separates the femoral implant and the tibial tray implant, which prevents the implants from rubbing together and causing wear spots due to friction. The polymeric tibial insert also absorbs and disperses the pressure imposed by a person's weight.

Generally, after inserting the components, the surgeon tests the knee joint's range of motion intraoperatively by elevating and lowering the knee, bending and extending the leg, and ensuring there are no gaps between the femoral and tibial implants. Testing the joint's range of motion ensures the implants have not been mal-aligned, which, as described above, could lead to adverse post-surgical complications.

Subsequent to testing the implant prosthesis, the implant components are removed and prepared for permanent insertion. Typically, cement is applied to desired portions of the components, which are then re-inserted and fixed into their permanent positions. The cement is allowed to harden, and range of motion tests are then performed again before the incision is closed and surgery is complete.

In one embodiment, and as shown in FIG. 2, sensor 10, which comprises at least one discrete sensing point 12, can be used in conjunction with an artificial joint implant to provide quantitative data for contact between bones and an implant during orthopedic implant surgery. It is contemplated that the sensor can also indirectly read the pressures, strains, and forces that the soft tissue places on the implant. A surgeon performing a joint replacement procedure can use this data to make necessary adjustments to the implants, bones, and associated tissue while performing the procedure, thereby reducing the risk of post operative complications. In one preferred aspect, the sensor can comprise the polymeric tibial insert.

In one exemplary method of using the sensor 10 to measure joint characteristics during revision joint replacement surgery, the joint is prepared for implant insertion and the joint replacement implant components, such as, for example, the femoral implant and the tibial tray implant and the sensor 10 in the form of the polymeric trial tibial insert, are positioned within the joint. The joint is then articulated through a partial or full range of motion. The force/pressure exerted on the sensor throughout the movement range is sensed and displayed or otherwise conveyed to the surgeon, who may then adjust the size or position of the implants and/or conduct the tissue balancing process based on the sensed pressure data. This sensing/adjustment cycle can be repeated as necessary to achieve a desired balance and alignment within the joint. Once no further adjustments are needed, the surgeon can remove the sensor (i.e., in this example, the trial tibial insert), re-insert a conventional tibial insert, fix the joint replacement implant into position, and close the incision. Optionally, it is contemplated that the joint replacement implant can be fixed into position using the trial tibial insert as the permanent tibial insert.

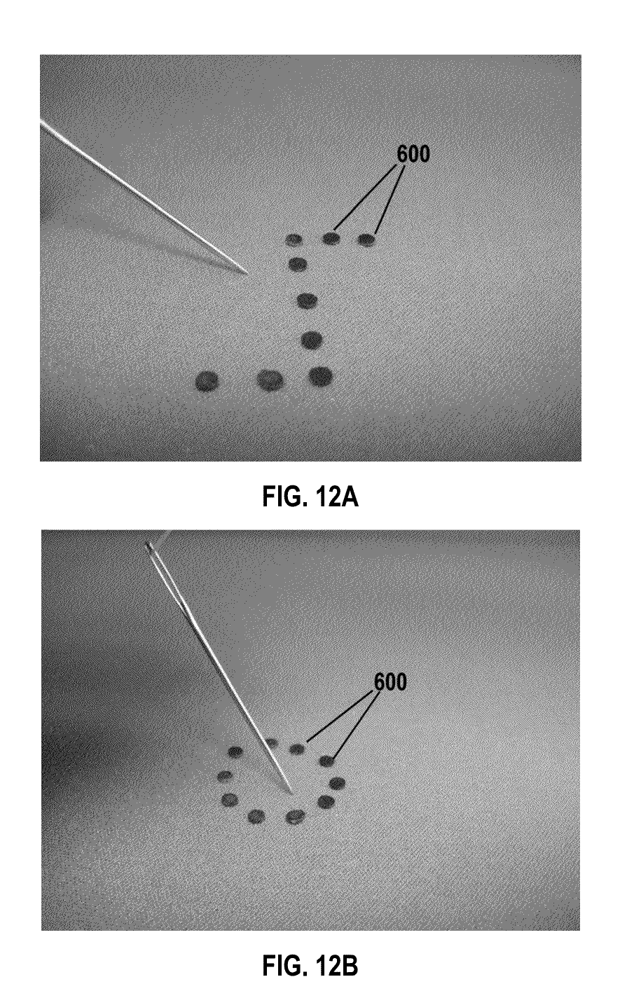

As noted herein, it is contemplated that the disclosed sensors can be molded in any desired size and shape. For example, in one aspect, and as shown in FIGS. 12A-12B, it is contemplated that the sensors can comprise sensing dots 600 that are molded to have diameters ranging from about 0.05 inches to about 0.35 inches, and more preferably between about 0.1 inches to about 0.2 inches and thicknesses of between about 0.010 inches to about 0.100 inches, and preferably about 0.025 inches. In this aspect, it is contemplated that the sensing dots 600 can comprise PPS and carbon black in an amount corresponding to a weight percentage ranging from between about 0.5% to about 30%, and preferably between about 1% to about 10% 1% to about 10% for each sensing dot. It is further contemplated that the sensing dots 600 can be operatively sandwiched between electrodes and wired to electronic analysis equipment, to provide for individual sensing points. In one example, a sensing dot 600 formed as discussed above and having a diameter of 3 mm can have a minimum pressure measurement scale of 0.5 psi (3.5 Pa) and permit full scale measurements of up to 70 lbf (311 kN). In one exemplary use, it is contemplated that the sensing points of the trial tibial insert can comprise sensing dots that are drilled or otherwise secured to the tibial insert so as to form a three-dimensional pressure mapping array. However, it is further contemplated that the sensing dots can be integrated into any known load-bearing device.

The contact sensors described herein may be better understood with reference to the Examples set forth below.

### Example 1

An industrial-grade UHMWPE powder (GUR 1150, available from Ticona Engineering Polymers) having a molecular weight of 6×10⁶, density of 0.93 g/mL, Tm of 135° C., and an average particle size of 100 μm, was combined with carbon black (CB) (Printex L-6 available from Degussa Hulls, Dusseldorf, Germany) having a primary particle size of 18 nm and dibutyl phthalate absorption of 120 mL/100 g. Amounts of each powder were placed in a 120 mL plastic sample container and initially manually shaken for 5 minutes to obtain four different samples having CB weight percentages of 0.25%, 0.5%, 1%, and 8%. The samples were then mixed for 10 minutes on a common laboratory vortex at the maximum speed setting.

Virgin UHMWPE powder and the four UHMWPE/CB powder mixtures were then compression-molded into rectangular sheets 12 cm long, 8.5 cm wide, and 2 mm thick using a mold consisting of a 2 mm thick Teflon frame sandwiched between 2 stainless steel plates that were coated with Teflon mold release spray. The powders were processed in a laboratory press (Carver Laboratory Press, Model C, Fred S. Carver Inc., Wabash, Ind.) equipped with electric heaters for 20 minutes at a temperature of 205° C. and a pressure of 10 MPa. The specimens were then quenched under pressure at a cooling rate of 50° C./min.

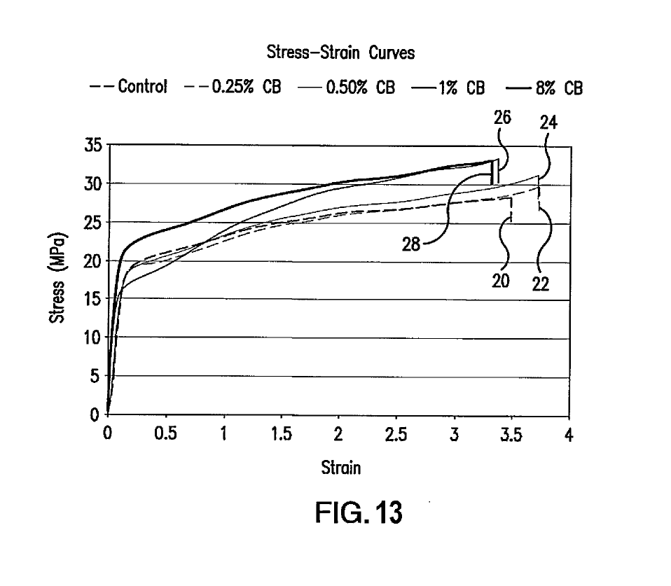

Tensile tests were performed to obtain stress-strain curves for each composite and for the control. Results can be seen in FIG. 13 for the control (20), 0.25% CB (22), 0.50% CB (24), 1.0% CB (26), and 8.0% CB (28). From these stress-strain curves, the modulus of elasticity was determined for each composite, and these values were compared to those obtained for the control specimen. Both the control specimens and the composite specimens were formed from the same stock of virgin UHMWPE powder (GUR 1150) by using the same processing parameters of temperature, pressure, time, and cooling rate. The results from the tensile tests can be seen in Table 1, below. It was determined that there was no statistically significant difference (p=0.32, β=0.05) between the modulus of the 8% composite and the modulus of the virgin UHMWPE control samples that were tested.

TABLE 1

| n = 4 | Control | 0.25 wt % CB | 0.50 wt % CB | 1 wt % CB | 8 wt % CB |
|---|---|---|---|---|---|
| Young's | 214.8 ± 21.1 | 208.48 ± 7.68 | 211.9 ± 7.74 | 212.6 ± 6.82 | 208.9 ± 11.1 |
| Modulus (MPa) | | | | | |
| Tensile | 30.8 ± 3.98 | 29.1 ± 2.23 | 32.6 ± 3.49 | 31.9 ± 2.43 | 31.7 ± 1.03 |
| Strength (MPa) | | | | | |
| Yield Strength | 17.8 ± 0.75 | 18.0 ± 0.87 | 17.8 ± 0.93 | 15.2 ± 0.96 | 22.2 ± 1.07 |
| (MPa) | | | | | |
| Elongation at | 390 ± 77.0 | 360 ± 18.0 | 390 ± 18.0 | 340 ± 23.0 | 290 ± 41.0 |
| Break (%) | | | | | |

The elastic modulus values obtained were comparable to those obtained by Parasnis and colleagues for thin-film UHMWPE specimens (see Parasnis C, Ramani K. Analysis of the effect of pressure on compression molding of UHMWPE. Journal of Materials Science: Materials in Medicine, Vol. 9, p 165-172, 1998, which is incorporated herein by reference). The values obtained for tensile strength, yield strength, and elongation at break compared closely to the values cited in the literature (for example, see is Li S. Burstein A. H., Current Concepts Review: Ultra-high molecular weight polyethylene. The Journal of Bone and Joint Surgery, Vol. 76-A, No. 7, p 1080-1090, 1994, which is incorporated herein by reference).

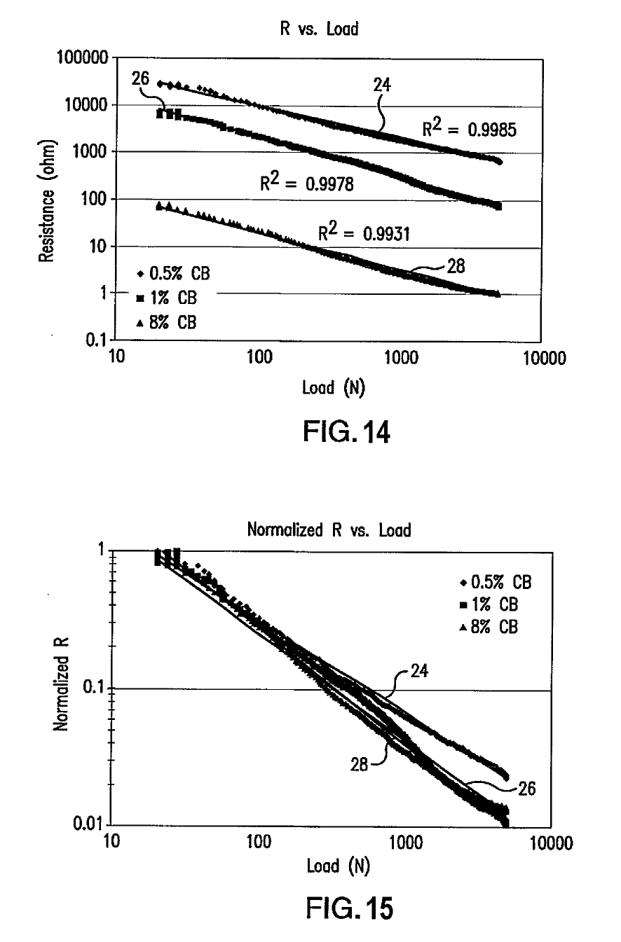

FIG. 14 shows a plot of the log of the resistance as a function of the log of the compressive load applied to the UHMWPE/CB composites of 0.5% (24), 1% (26), and 8% (28). The plot shows that the composites have the same slope, but that the intercepts are different, with the 0.5% composite having the highest intercept, and the 8% composite having the lowest intercept. The value of resistance changed by about two orders of magnitude for each composite. The correlation coefficients of each regression line indicated a good fit. When the values of resistance were normalized (shown in FIG. 15), the curves for the three composites were very similar, suggesting that the amount of CB only affected the magnitude of the resistance. Thus, the relative response to applied load appeared to be independent of the amount of CB. It should be noted that the control sample and the 0.25% CB sample had high resistance for all loads tested and thus were not included on FIGS. 14 and 15.

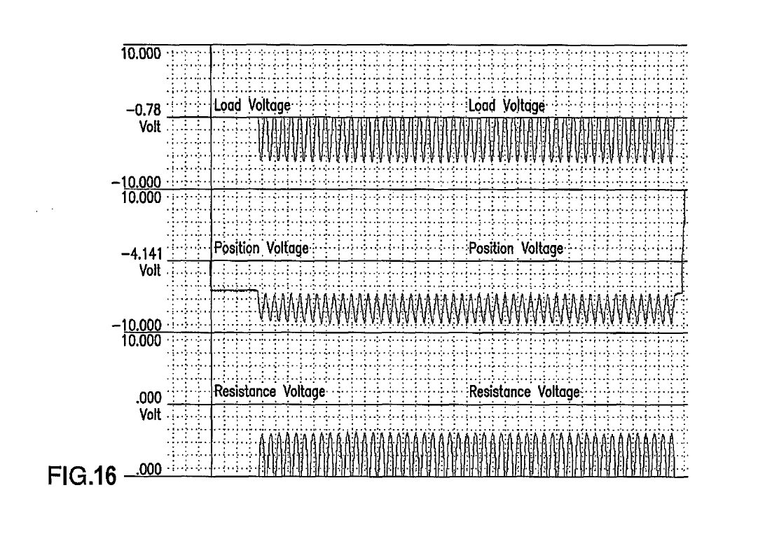

FIG. 16 shows the voltage values corresponding to the compressive load, the compressive displacement, and the resistance of the 8 wt % CB composite while the composite was loaded cyclically with a haversine wave at 1 Hz. The top curve corresponds to the compressive stress, the middle curve corresponds to the compressive strain, and the bottom curve corresponds to the resistance of the sensor material. This data represents the cyclic response of the material, indicating that it does not experience stress-relaxation at a loading frequency of 1 Hz. The results of this cyclic testing show that the peak voltage values corresponding to resistance remain nearly constant over many cycles. Therefore, the data seem to indicate that the sensor material should be well suited for cyclic measurements since the readings do not degrade over time.

Monitoring the electrical resistance of the composite material while applying a compressive load revealed the force-dependent nature of the electrical properties of the material. Because of the nano-scale dispersion of the conductive filler, the material's electrical response to applied load was nearly ideal for all of the percentages tested. That is, the log of the material's resistance varied linearly with respect to the log of the applied load. This linear relationship makes the material well suited for use as a sensor.

The data show that the linear relationship holds true for 0.5%, 1%, and 8%, with the difference between the three being the value of the resistance. As all three percentages showed good sensor properties, specific formulations could be developed based on other criteria, such as the specifics of the measurement electronics.

### Example 2

Compression molding was used to form 2 rectangular blocks of 1150 UHMWPE doped with 8 wt % carbon black filler as described above for Example 1. The blocks formed included a 28×18 matrix of surface sensing points 12 as shown in FIG. 2. The points were circular with a 1/16^{th} inch (1.59 mm) diameter and spaced every 1/10^{th} inch (2.54 mm) The blocks were then machined to form both a highly-conforming, PCL-sacrificing tibial insert (Natural Knee II, Ultra-congruent size 3, Centerpulse Orthopedics, Austin, Tex.) and a less conforming PCL-retaining tibial insert (Natural Knee II, Standard-congruent, size 3, Centerpulse Orthopedics, Austin, Tex.) as illustrated in FIG. 2. The implants were then aligned and potted directly in PMMA in the tibial fixture of a multi-axis, force-controlled knee joint simulator (Stanmore/Instron, Model KC Knee Simulator). Static testing was performed with an axial load of 2.9 kN (4.times.B.W.) at flexion angles of 0°, 30°, 60°, and 80°, to eliminate the effects of lubricant and to compare the sensor reading to the literature. The dynamic contact area was then measured during a standard walking cycle using the proposed 1999 ISO force-control testing standard, #14243. Data was collected and averaged over 8 cycles. A pure hydrocarbon, light olive oil was used as the lubricant due to its inert electrical properties.

Static loading of the sensors showed that the contact area of the ultra congruent insert was significantly higher than that of the standard congruent insert at all angles of flexion tested. The data closely agreed with results found in the literature from FEA analysis.

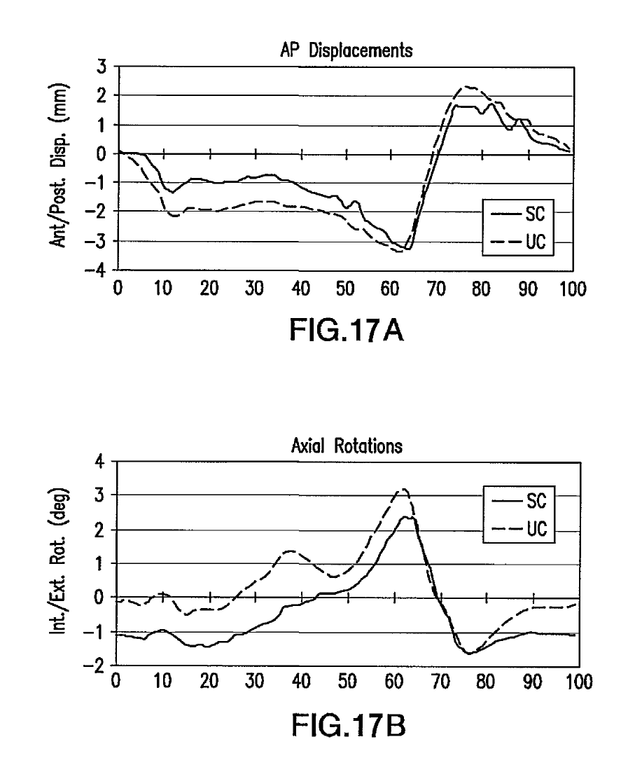

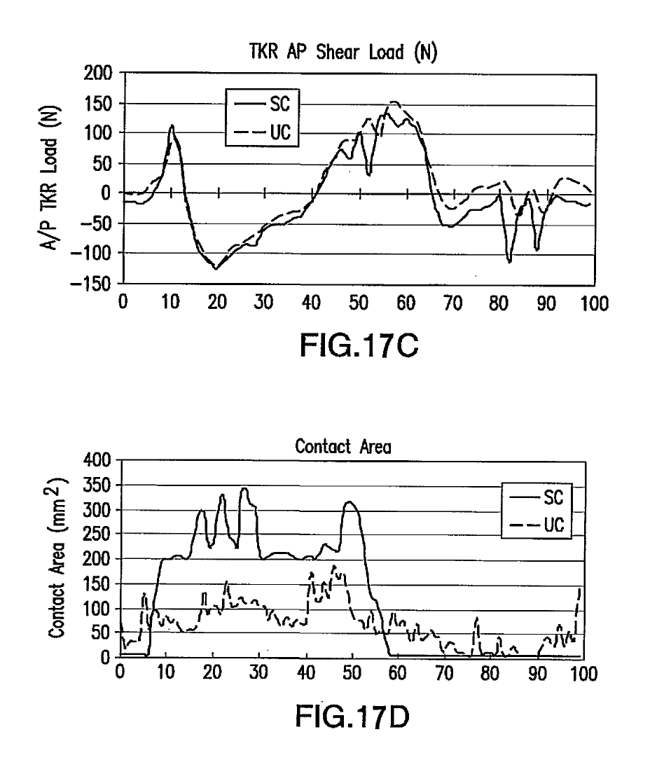

The results from dynamic testing with a standard walking protocol, shown in FIG. 17A-17D, show the effects that the lubricant had on the dynamic contact area. Contact area was registered by the sensor when physical contact occurred between the femoral component and any sensing point, allowing electrical current to flow. Because the lubricant was electrically insulating, fluid-film lubrication over a sensing point caused no contact to be registered at that point. The lower contact area measured for the ultra-congruent insert during the stance phase of gait was due to the fluid-film lubrication that occurred with the more conforming insert. The rapid changes in contact area measured for the standard-congruent insert during the mid-stance phase suggests that the mode of lubrication is quite sensitive to the dynamic loading patterns.

### Example 3

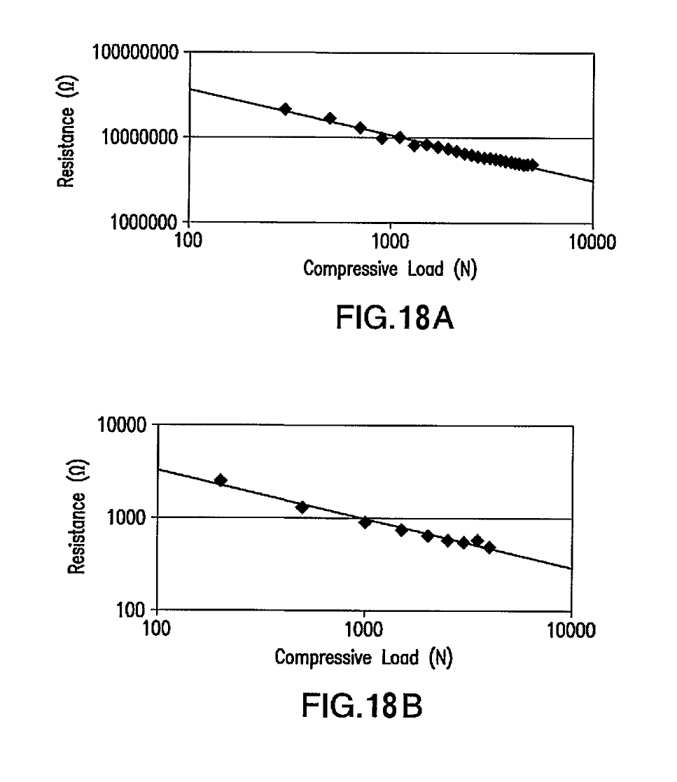

Tecoflex SG-80A, a medical grade soft polyurethane available from Thermedics Inc. (Woburn, Mass.), was solution processed and molded including 4 wt % and 48 wt % CB to form two solid sample materials. FIGS. 18A and 18B graphically illustrate the resistance vs. compressive force applied to the samples for the 4% and 48% non-surfactant mixed samples, respectively. As can be seen, both samples showed pressure sensitive conductive characteristics suitable for forming the sensors as described herein where the value of resistance can be controlled with the amount of conductive filler added.

### Example 4

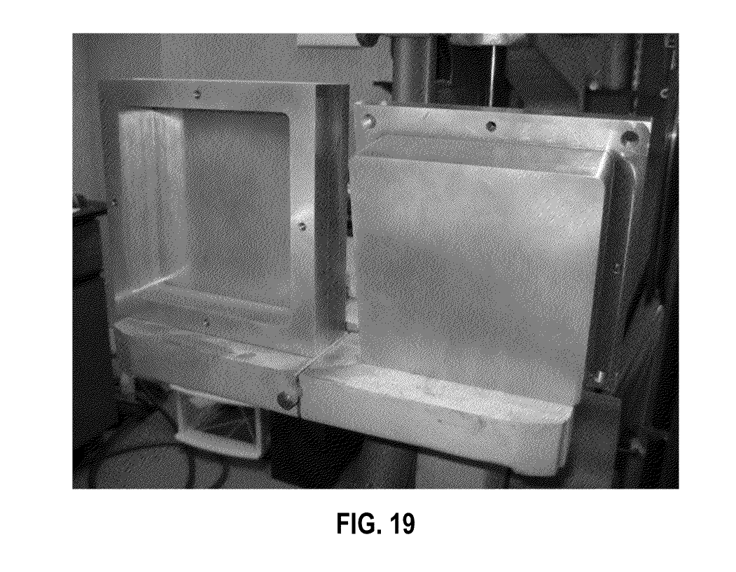

A 6″×6″ mold was constructed from normalized, pre-hardened 4140 steel with a Rockwell hardness of HRC 32-35. The mold was designed and built to mold 6×6 inch sensor sheets at approximately ⅛^{th} inch thick, and is shown in FIG. 19.

The mold was used to form “virgin” non-conductive sheets from raw high density polyethylene (HDPE) in powder form, similar to the fashion to form the sheets of UHMWPE in Example 1. HDPE works well in applications in which the sensor sheets need to be thermoformed. However, HDPE's low gel viscosity makes it a challenge to keep adjacent regions of the sensor sheet separated from one another when forming the sensor sheet.

Although HDPE's melt temperature is readily available, observations were made to confirm how the sensor sheets would behave in the mold. A thermocouple was used to measure the temperature in the oven. It was determined that the transition temperature of the HDPE was 255° Fahrenheit. The mold was heated by upper and lower platens, which also apply compressive force. It was noted that, even with the correct temperature being applied, some smearing could occur in the sensor sheet if compressive forces were not applied evenly. Any flow of the gel caused smearing.

Once the virgin sheets were constructed, the conductive regions were added and the sheets were placed back into the same mold. Since neither the mold nor the press were perfectly square, the sheets that were produced varied by 10-20 thousands of an inch. To minimize these variances, the mold sections and the press sections were labeled on each corner. Once the mold was labeled, different mold and press alignments were tested to determine the alignments the produced the sheets with the least variance.

Raw material in powder form was melted in the mold under the optimal alignment determined previously to produce the virgin sensor sheet. The conductive filler portions were placed on the virgin sensor sheet. Then, the sheet with the conductive filler was placed back into the mold with the same alignment to ensure that any dimensional variance that were present during the initial molding will also be present for the second molding. It was found that this procedure reduced material flow inside the mold during gel state, which reduced smearing.

Force/Pressure Sensors

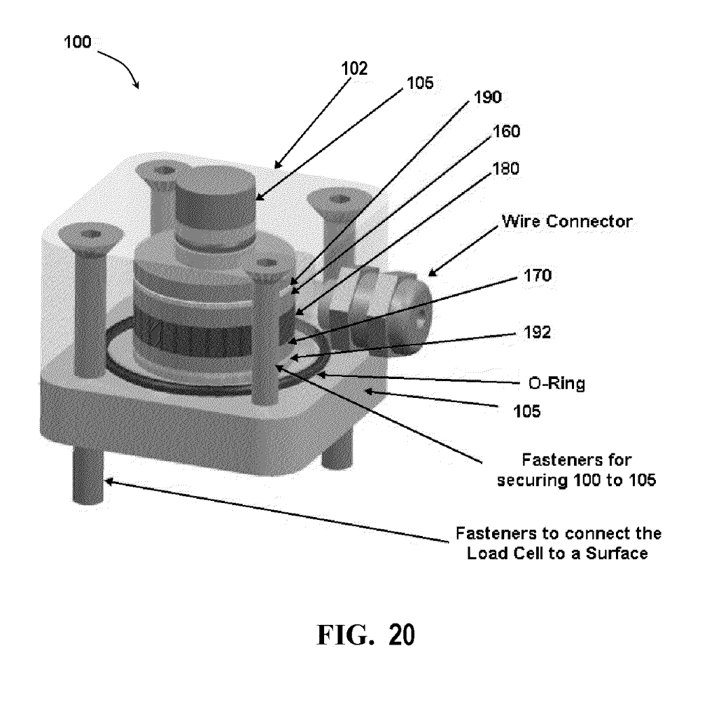

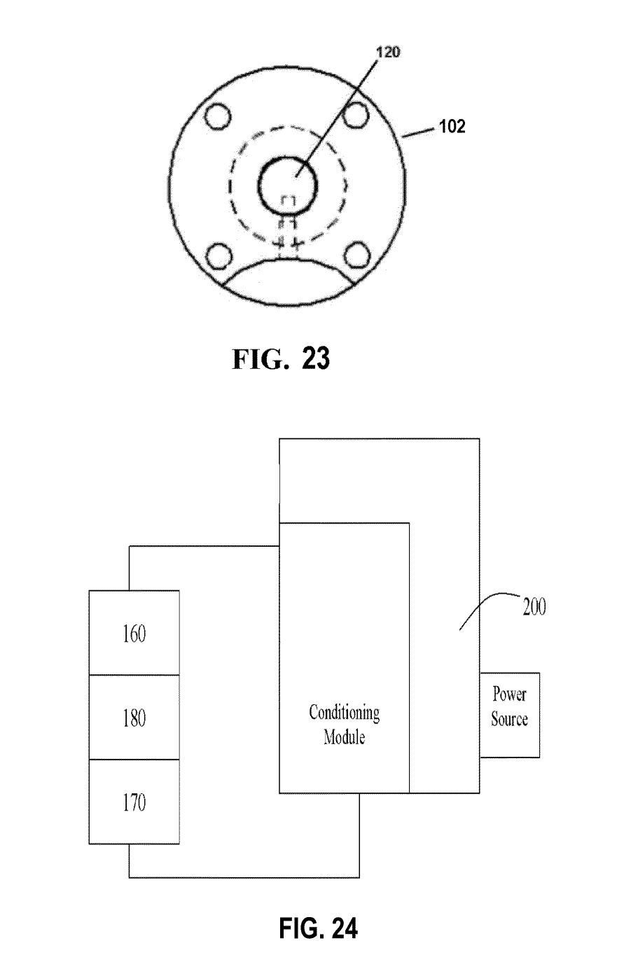

In one exemplary aspect of a force/pressure sensor, presented herein are aspects of a load cell 100. In one aspect, and with reference to FIGS. 20-23, the load cell 100 comprises a load knob 150. A distal end of the load knob 150, for example, can be connected to a load member 140, which can optionally be formed integral with the load member. Optionally, the load cell 100 can comprise a load cell housing 102. In one aspect, the load cell housing 102 can define an interior cavity 110. In this aspect, the load cell housing 102 can also define a bore 120 in a first exterior face 130 of the load cell housing. In another aspect, the load member 140 can be positioned within the interior cavity 110 of the load cell housing 102. In this aspect, a proximal end of the load knob 150 can protrude out of the bore 120 and above the first exterior face 130 of the load cell housing 102. As shown, in one aspect, it is contemplated that the load knob is configured to cooperate with the bore 120 of the load cell housing such that the load knob can move axially relative to the first exterior face 130 of the load cell housing 102. For example, a load impacting or placed thereon the load knob can cause the lad knob to translate axially and impart a like compressive force, via the distal end of the load knob, on portions of the load cell that underlie and are otherwise in operative contact with the load knob.

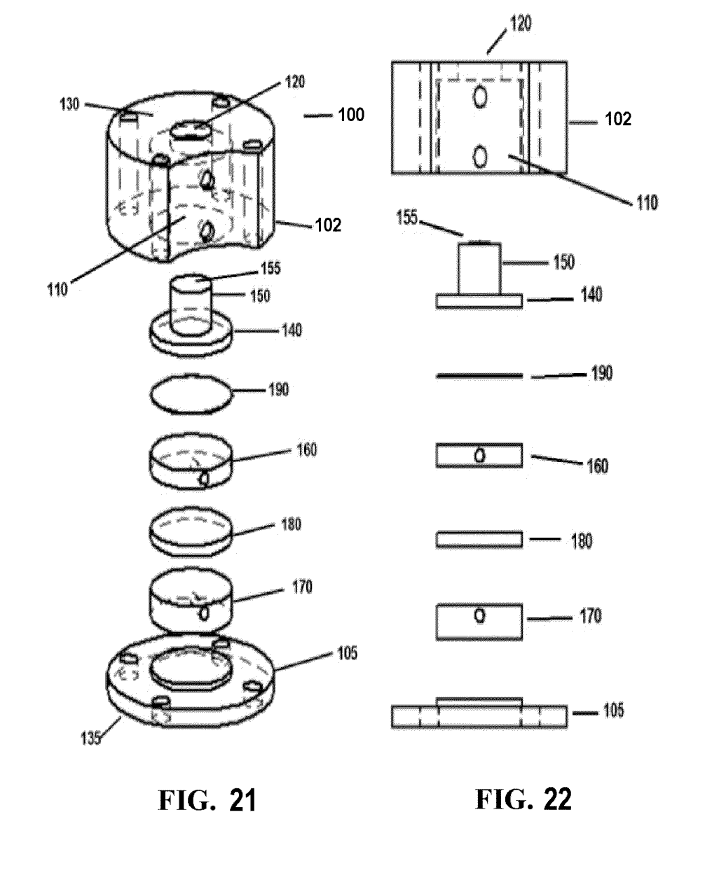

In one aspect, the load cell 100 further comprises a first electrode 160 and a second electrode 170 positioned within the interior cavity 110 of the load cell housing 102. In another aspect, a conductive polymer element 180 substantially separates the first and second electrodes 160, 170. In this aspect, the first electrode 160 can substantially underlie the load member, and the second electrode 170 can substantially overlie a second exterior face 135 of the load cell housing 102, which opposes the first exterior face 130, as illustrated in FIG. 21. In one aspect, and as described in more detail below, the conductive polymer element is substantially inflexible. Optionally, the polymer element is substantially planar and is positioned in substantially uniform contact with the respective faces of the first and second electrodes. In one exemplary aspect, the conductive polymer element can have a disk shape, however, any other geometric shape will suffice.

In operation, in one aspect, an excitation voltage is operably applied to the load cell 10 via the first and second electrodes 160, 170. The conductive polymer element 180 between the two electrodes 160, 170 completes an electrical circuit. An exemplary schematic of the electrical circuit is shown in FIG. 24.

In operation, when a compressive force is applied to the load knob 150, the load is transferred to the first and second electrodes 160, 170 and conductive polymer element 180, which effects a compression of the conductive polymer element. As the compressive force increases, the current flow through the conductive polymer element 180 from the first electrode 160 to the second electrode 170 increases because the resistance in the conductive polymer element 180 decreases. Alternatively, when a tensile force is applied to the load knob 150, the resistance in the conductive polymer element 180 increases, thus reducing the current flow. In this aspect, the load cell can be pre-loaded and calibrated to measure both compressive and tensile forces. This current flow can be measured by conventional means and converted to engineering units to calculate a load cell output.

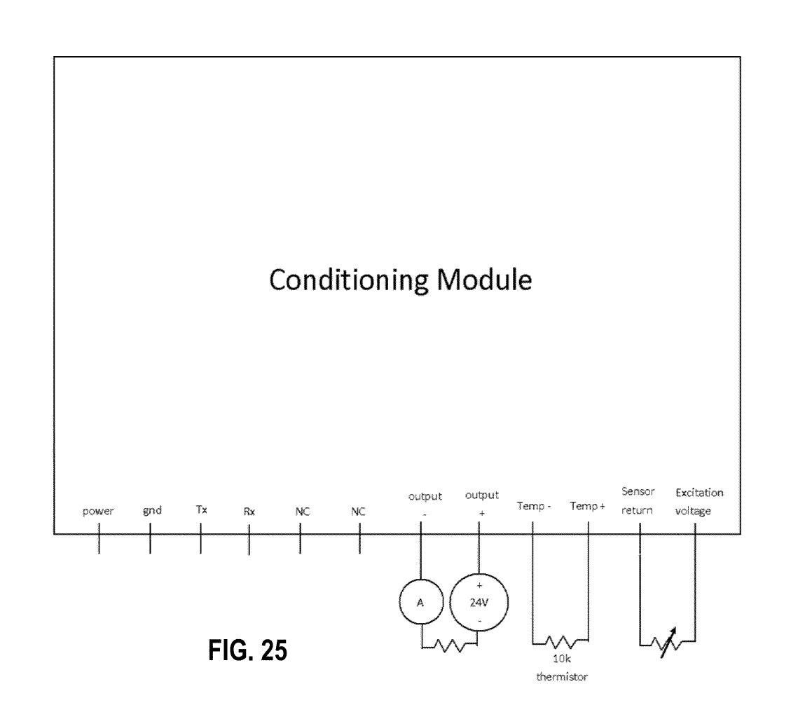

In another aspect, the measured load cell output can be communicated to a conditioning module for electrical processing. A schematic of an exemplary conditioning module is shown in FIG. 25. It is contemplated that the load cell output can be substantially non-linear. In one aspect, the conditioning module can comprise a microcontroller configured to convert the measured load cell output into a substantially linear output (the converted load cell output) that can be processed by conventional data collection terminals. In this aspect, it is contemplated that the load cell output can range from about 4 mA to about 20 mA. It is further contemplated that the converted load cell output can be displayed on a light-emitting diode (LED) readout or other conventional display means.

In an additional aspect, the conditioning module can comprise a shunt resistor in electrical communication with the first and second electrodes 160, 170 and the conductive polymer element 180 of the load cell 100. In this aspect, the shunt resistor can have a resistance ranging from about 2 Ohms to about 10,000 Ohms, more preferably ranging from about 10 Ohms to about 1,000 Ohms, and most preferably ranging from about 100 Ohms to about 300 Ohms. In a further aspect, the conditioning module can comprise an analog/digital converter (A/D converter) for measuring the voltage drop across the shunt resistor. In this aspect, the A/D converter can be in communication with the microcontroller to digitally filter and display the converted load cell output. Optionally, the converted load cell output can be transmitted through a digital/analog (D/A converter) to output a substantially linear signal that can be read by conventional industrial data collection terminals, thereby permitting electrical interaction with other conventional industrial equipment. For example, it is contemplated that the load cell can be used in a feedback loop to control the operation of a conventional industrial device based on the load cell output.

In still a further aspect, the conditioning module can be powered by a power source. In this aspect, the power source of the conditioning module can be a low voltage power source. It is contemplated that the power source can provide a voltage of 24 Volts (DC) or another common voltage available in conventional industrial settings. It is further contemplated that the load cell output, prior to conversion, can have a substantially greater amplitude than the outputs of conventional load sensors, thereby reducing the susceptibility of the load cell output to noise and other sources of interference.

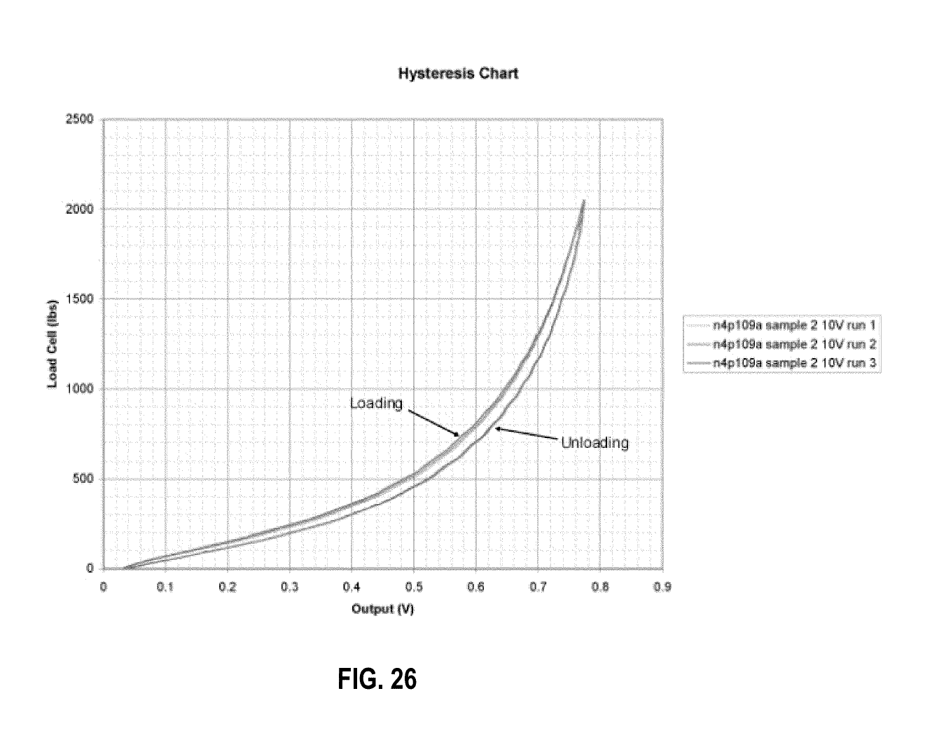

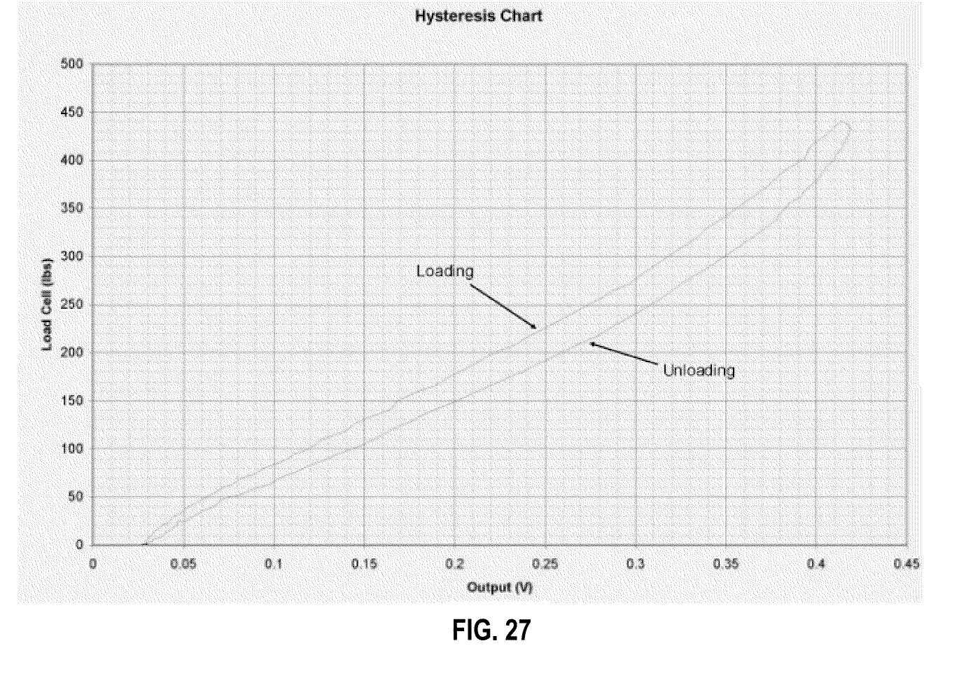

In one exemplary aspect, as shown in FIGS. 26-27, it can be appreciated that, upon application of a load, the potential measured across the conductive polymer element increases. As shown in FIGS. 26-27, at least initially, the output increases substantially linearly. As the load increases, the measured output increases at a greater rate, as illustrated on the graph of FIGS. 26-27, where the slope of the line representing output increases with greater load. As one can appreciate, these load graphs can be used to calibrate the load cell.

Additionally, as illustrated in FIGS. 26-27, the characteristics of the load versus output graph indicate that, after loading, and upon unloading, the load cell can experience hysteresis. Thus, the signal processing component necessary for correlating the voltage or current to the load can be implemented using software capable of correlating the load during loading to the output according to the loading portion of the graph, and correlate the load during unloading to the unloading portion of the graph. In another aspect, the software can calculate the load during static loading (i.e. at a point at which the load is constant) by estimating a point between the loading portion of the graph and the unloading portion of the graph.

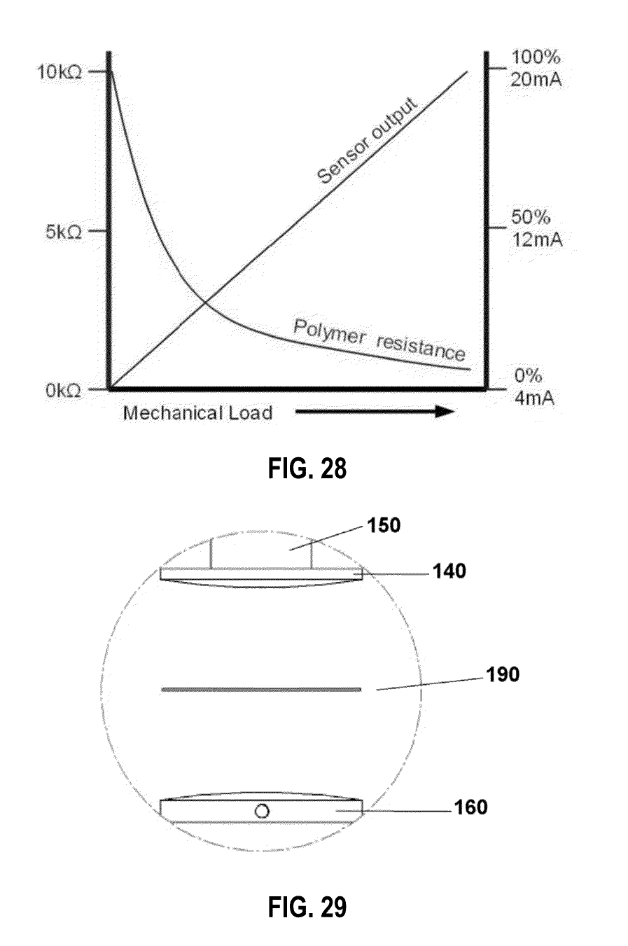

In another aspect, as depicted in FIG. 28, it is contemplated that the conductive polymer element of the load cell can have greater sensitivity at smaller loads than at larger loads. This greater sensitivity at smaller loads translates into a sharp drop in the resistance of the conductive polymer element as the load increases. Accordingly, it is contemplated that the load cells described herein can produce outputs at higher resolutions than conventional strain gauge load cells. In particular, it is contemplated, in a comparison between a load cell described herein and a conventional strain gauge load cell, where both load cells have equal maximum loading capabilities (full scales), the load cell described herein can have superior accuracy from about 0.001% full scale to about 10% full scale of the load cells. Thus, a 1,000 pound load cell as described herein can have greater accuracy than a 1,000 pound conventional strain gauge load cell at loads ranging from about 0.01 pounds to about 100 pounds.

In a further aspect, the load cell can be configured to measure dynamic loads in addition to static loads. In this aspect, the load cell can have a response time indicative of the time between transfer of a load to the load cell and generation of the load cell output. It is contemplated that the response time of the load cell can range from about 1 microsecond to about 10 microseconds. However, it is contemplated that the load cell can have other response times as desired depending on the end use of the load cell. The response time of the load cell can closely approximate the response times of conventional piezo-electric load cells, which are regularly used within the art to measure dynamic loads. Thus, the load cells described herein can be used to perform measurements of dynamic loads. However, unlike conventional piezo-electric load cells, the load cells described herein can also accurately measure static loads, eliminating the need for a separate load cell, such as a conventional strain gauge. Therefore, the load cells described herein can be used to accurately conduct measurements of both dynamic and static loads.

In one aspect, at least a portion of the exterior surface 155 of the proximal end of the load knob 150 can comprise an arcuate surface. In another aspect, the exterior surface 155 of the load knob 150 is semi-spherical. In this aspect, forces directed onto the exterior surface 155 of the load knob 150 are substantially axially transferred to the first electrode and tangential forces are minimized.

In another exemplary aspect, at least a portion of the distal end of the bottom portion of the load member can be substantially convex, as shown in FIG. 29. In this aspect, a top portion of the first electrode may also be substantially convex. Thus, a load applied to the load member that is not axial to the first electrode would be translated substantially axially. Alternatively, at least a portion of the exterior surface of the load knob may be connected to a portion of the load member pivotally, such that, as a non-axial force is applied to the load knob, at least a portion of the applied forces are directed axially to the first conductor and, thus, can be calibrated.

In another aspect, a first insulator 190 can be positioned between the load member 140 and the first electrode 160. In this aspect, a second insulator 192 can be positioned between the second electrode 170 and the lower housing 105. The respective first and second insulators 190, 192 can comprise, for example and not meant to be limiting, polytetraflouroethylene (“PTFE”). In one aspect, the load cell housing can comprise a low friction material, such as for example, ultra high molecular weight polyethylene (“UHMWPE”).

In another aspect, the load cell can comprise a thermistor that is configured to change it's resistance in response to temperature. In one aspect, it is contemplated that the thermistor can be positioned within the load cell housing. In operation, the thermistor reads the temperature inside the load cell housing and compensates the output based on the sensed temperature. When the temperature increases, the output increases, so the microcontroller compensates for that artificial increase by artificially decreasing the output such that at a constant force, the load cell will read the same force regardless of what the load cell's temperature is. Generally, the controller or computer can use a gain value to multiply all the lookup table values depending on the temperature measured at any given moment.

In one aspect, and with reference to FIGS. 20-23, the load cell housing 102 comprises a substantially cylindrical shape, while the internal components within the interior cavity (i.e. the electrodes, the conductive polymer element, and the insulator) can comprise a complementary disc shape. In this aspect, the tolerances between the internal components and the load cell housing 102 are substantially tight in order to allow the parts to transfer force with very little motion. In another aspect, the internal components can have an outside diameter ranging from about 0.500 inches to about 1.500 inches. For example, and without limitation, the internal components can have an outside diameter of between about 0.500 inches to about 2.000 inches, and preferably about 1.000 inches. In an additional aspect, the load cell housing 102 can have an inner diameter ranging from between about 0.300 inches to about 1.7 inches, and preferably about 0.500 inches to about 1.500 inches. For example, and without limitation, the load cell housing 102 can have an inner diameter of about 1.010 inches. In a further aspect, the internal components can have a thickness ranging from between about 0.020 inches to about 0.500 inches, more preferably from between about 0.050 inches to about 0.350 inches.

In an additional aspect, the conductive polymer element can be configured to withstand a maximum pressure before a pressure overload occurs, at which point the conductive polymer element loses calibration and plastically deforms. In this aspect, it is contemplated that the maximum pressure that the conductive polymer element can withstand can be about 12,000 pounds per square inch. In a further aspect, it is contemplated that the load cells described herein can be configured to withstand overloads ranging from between about 2 times full scale to about 15 times full scale, more preferably ranging from between about 4 times full scale to about 12 times full scale. It is further contemplated that the diameter—and cross-sectional area—of the internal components within the interior cavity of the load cell housing 102 can be increased to provide additional overload protection. For example, and without limitation, a load cell as described herein having internal components with a diameter of 1 inches and a full scale of 1,000 pounds can withstand a load of approximately 10,000 pounds. However, if the diameter of the internal components was increased, then the load cell could withstand an even greater load.

In a further aspect, the load cells described herein can have a zero balance indicative of the load cell output when no load is applied. In this aspect, and with reference to FIGS. 20-23, it is contemplated that because the conductive polymer element 180 has only minimal with other internal components of the load cell 100 when no load is applied, there is substantially no current flowing through the sensor. Consequently, when no load is applied to load cell 100, there will be substantially no load cell output. In contrast, strain gauge sensors and other conventional load cells can have zero balances ranging from about 1% to about 5% of full scale.

In yet another aspect, the second exterior face 135 of the load cell housing is attached to the load cell housing a plurality of fasteners, such as screws. In one aspect, a lower housing 105 comprises the second exterior face. In this aspect, a portion of the lower housing 105 protrudes into the interior cavity of the load cell housing 102. In this aspect, tightening of the fasteners secures the lower housing onto the load cell housing and provides a compressive pre-load for the internal components. In this aspect, the load knob can be compressed to measure compressive force, or the load knob may be pulled, measuring tensile force.

In one aspect, the conductive polymer element 180 can include an electrically conductive pressure sensitive composite material. In general, any polymeric material that can be combined with an electrically conductive filler to form a pressure sensitive conductive polymeric composite material that can then be formed into an essentially inflexible shape can be utilized for the conductive polymer element. For instance, various polyolefins, polyurethanes, polyester resins, epoxy resins, and the like can be used. In certain aspects, the composite material can include engineering and/or high performance polymeric materials. In one aspect, the composite material can include polyphenolyne sulfide (“PPS”). PPS comprises a high modulus of elasticity, which is beneficial for maintaining dimensional stability under load. In another aspect, the composite material can include UHMWPE. UHMWPE is generally classified as an engineering polymer, and possesses a unique combination of physical and mechanical properties that allows it to perform extremely well in rigorous wear conditions. In fact, it has the highest known impact strength of any thermoplastic presently made, and is highly resistant to abrasion, with a very low coefficient of friction. As can be appreciated, other thermoplastics with substantially similar characteristics can be used.

According to one aspect, a pressure sensitive conductive composite material can be formed by combining a desired amount of conductive filler with a polymeric material. In one aspect, the desired amount of conductive filler can range from about 0.2% to about 20% by weight of the composite material, more preferably from about 0.5% to about 10% by weight of the composite material, and most preferably from about 1% to about 3% by weight of the composite material. Of course, in other aspects, the composite material can include a higher weight percentage of the conductive filler material.

In general, the polymeric material and the conductive filler can be combined in any suitable fashion, which can generally be determined at least in part according to the characteristics of the polymeric material. For example, and depending upon the polymers involved, the materials can be combined by mixing at a temperature above the melting temperature of the polymer (conventional melt-mixing) and the filler materials can be added to the molten polymer, for instance, in a conventional screw extruder, paddle blender, ribbon blender, or any other conventional melt-mixing device. The materials can also be combined by mixing the materials in an appropriate solvent for the polymer (conventional solution-mixing or solvent-mixing) such that the polymer is in the aqueous state and the fillers can be added to the solution, optionally utilizing an appropriate surfactant if desired, following which the solvent can be allowed or encouraged to evaporate, resulting in the solid conductive composite material. In another aspect, the materials can be mixed below the melting point of the polymer and in dry form, for instance, in a conventional vortex mixer, a paddle blender, a ribbon blender, or the like, such that the dry materials are mixed together before further processing.

It is contemplated that, when mixing the components of the composite material, the mixing can be carried out under any suitable conditions. For instance, in one aspect, the components of the composite material can be mixed at ambient conditions. In other aspects, however, mixing conditions can be other than ambient, for example and without limitation, so as to maintain the materials to be mixed in the desired physical state and/or to improve the mixing process.

When dry mixing the materials to be utilized in the composite, the exact particulate dimensions of the materials are not generally critical to the invention. However, in certain aspects, the relative particulate size of the materials to be combined in the mixture can be important. In particular, the relative particulate size of the materials to be combined can be important in those aspects wherein a relatively low amount of conductive filler is desired and in those aspects wherein the polymer granules do not completely fluidize during processing. For instance, the relative particle size can be important in certain aspects wherein engineering or high-performance polymers are utilized, and in particular, in those aspects utilizing extremely high melt viscosity polymers such as UHMWPE, which can be converted via non-fluidizing conversion processes, such as compression molding or RAM extrusion processes.

In such aspects, the particle size of the filler can beneficially be considerably smaller than the particle size of the polymer. According to this aspect, and while not wishing to be bound by any particular theory, it is believed that due to the small size of the conductive filler particles relative to the larger polymer particles, the conductive filler is able to completely coat the polymer during mixing and, upon conversion of the composite polymeric powder in a non-fluidizing conversion process to the final solid form, the inter-particle distance of the conductive filler particles can remain above the percolation threshold such that the composite material can exhibit the desired electrical conductivity. According to this aspect, when forming the composite mixture, the granule or aggregate size of the conductive filler to be mixed with the polymer can be at least about one order of magnitude smaller than the granule size of the polymer. In some aspects, the granule or aggregate size of the conductive filler can be at least about five orders of magnitude smaller than the granule size of the polymer.

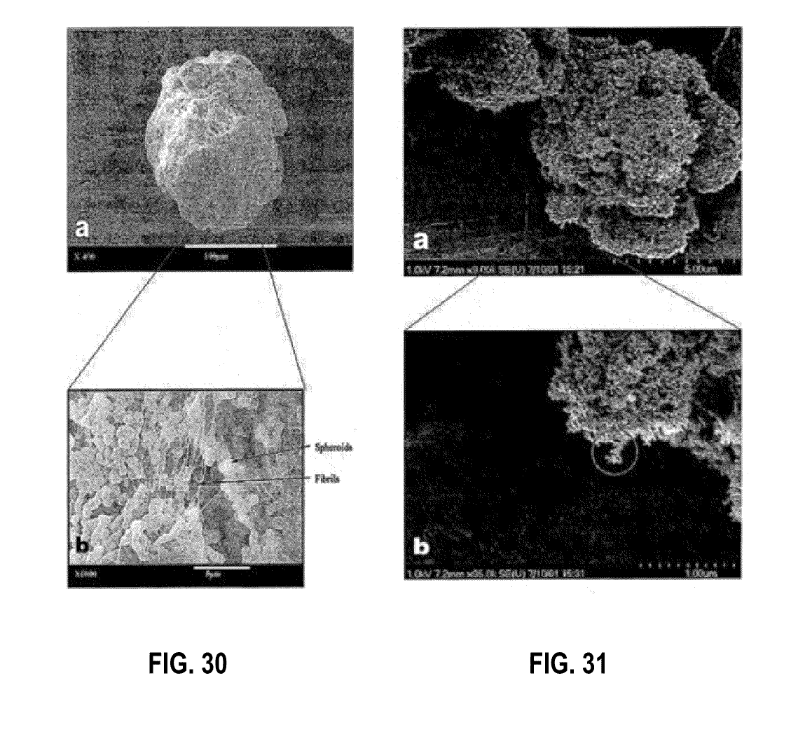

In forming the composite material according to this aspect, a granular polymer, such as, for example and not meant to be limiting, the UHMWPE illustrated in FIG. 30, can be dry mixed with a conductive filler that is also in particulate form. FIG. 30A is an FESEM image of a single UHMWPE granule. The granule shown in FIG. 30A has a diameter of approximately 150 μm, though readily available UHMWPE in general can have a granule diameter in a range of from about 50 μm to about 200 nm. FIG. 30B is an enlarged FESEM image of the boxed area shown on FIG. 30A. As can be seen, the individual granule is made up of multiple sub-micron sized spheroids and nano-sized fibrils surrounded by varying amounts of free space.

In one exemplary aspect, carbon nano-tubes or carbon nano-fibers can be used as the conductive filler to be mixed with the polymer. In another aspect, carbon black conductive filler can be mixed with the polymer. Carbon black is readily available in a wide variety of agglomerate sizes, generally ranging in diameter from about 1 μm to about 100 μm that can be broken down into smaller aggregates of from about 10 nm to about 500 nm upon application of suitable energy. For example, FIG. 31A is an FESEM image of a carbon black powder agglomerate having a diameter of approximately 10 nm. In FIG. 31B, individual carbon black aggregates forming the agglomerate can clearly be distinguished. The circled section of FIG. 31B shows a single carbon black aggregate loosely attached to the larger agglomerate. As the scale of FIG. 31B illustrates, the aggregates in this particular image range in size from about 50 nm to about 500 nm. In the circled section of FIG. 31B can be seen the smaller, spherical primary particles of carbon black, the size of which are often utilized when classifying commercial carbon black preparations. These primary particles make up the aggregate.

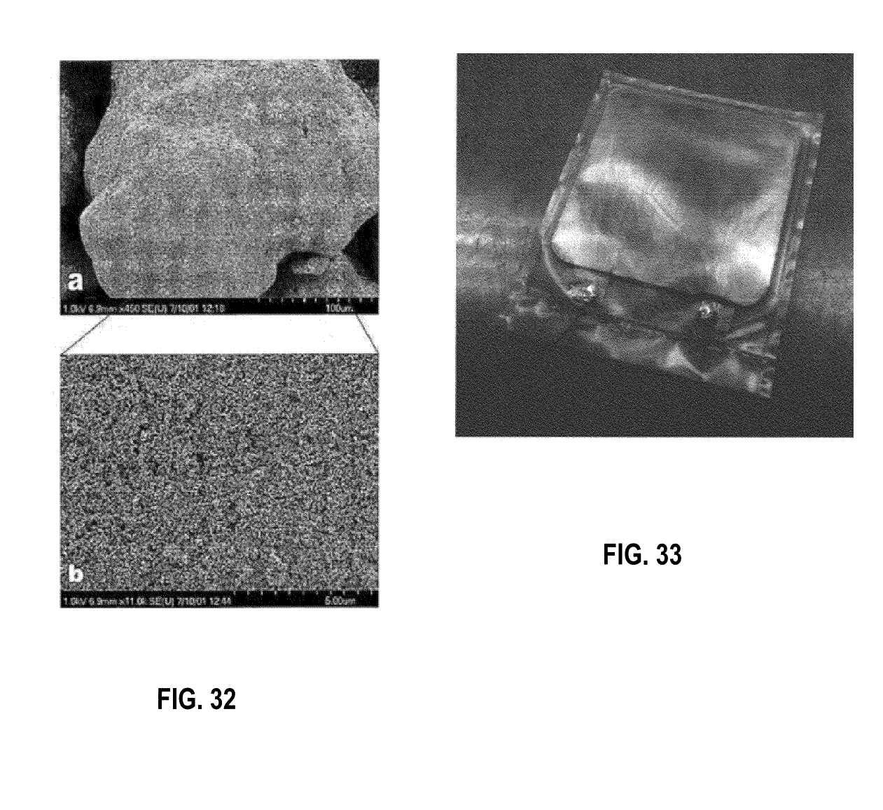

Upon dry mixing the particulate conductive filler with the larger particulate polymer material with suitable energy, the smaller granules of conductive filler material can completely coat the larger polymer granules. For instance, FIGS. 32A and 32B show FESEM micrographs of a single powder particle obtained following mixing of 8 wt % carbon black with 92 wt % UHMWPE. As can be seen, the UHMWPE particle is completely coated with carbon black aggregates. While not wishing to be bound by any particular theory, it is believed that forces of mixing combined with electrostatic attractive forces between the non-conductive polymeric particles and the smaller conductive particles are primarily responsible for breaking the agglomerates of the conductive material down into smaller aggregates and forming and holding the coating layer of the conductive material on the polymer particles during formation of the composite powder as well as during later conversion of the powdered composite material into a solid form.

Following formation of the mixture including a conductive filler and a polymeric material, the mixture can be converted as desired to form a solid composite material that is electrically conductive. The solid composite thus formed can also maintain the physical characteristics of the polymer in those aspects including a relatively low filler level in the composite. For example, in the aspect described above, in which the composite material includes a conductive filler mixed with UHMWPE, the powder can be converted via a compression molding process or a RAM extrusion process, as is generally known in the art, optionally followed by machining of the solid molded material, for instance in those aspects wherein a contact sensor describing a complex contact surface curvature is desired.

In other aspects however, and primarily depending upon the nature of the polymeric portion of the composite, other conversion methods may preferably be employed. For example and without limitation, in other aspects the polymeric portion of the composite material can optionally be a polymer, a co-polymer, or a mixture of polymers that can be suitable for other converting processes, and the composite polymeric material can be converted via, for instance, a relatively simple extrusion or injection molding process.

It is contemplated that the composite material of the disclosed sensors can optionally include other materials, in addition to the primary polymeric component and the conductive filler discussed above. Other fillers that can optionally be included in the disclosed composite materials of the present invention can include, for example, various ceramic fillers, aluminum oxide, zirconia, calcium, silicon, fibrous fillers, including carbon fibers and/or glass fibers, or any other fillers as are generally known in the art. In one aspect, the composite material can include an organic filler, such as may be added to improve sliding properties of the composite material. Such fillers include, for instance, tetrafluoroethylene or a fluororesin.

Optionally, a load cell and conditioning module as disclosed herein can be electrically connected in series with one or more conventional load sensors to form a hybrid load cell. In one aspect, the hybrid load cell can comprise a load cell and conditioning module as disclosed herein connected in series with a conventional strain gauge sensor. It is contemplated that the load cell as described herein and the strain gauge sensors can have equivalent full scale calibration values. In this aspect, the hybrid load cell can further comprise conventional strain gauge conditioning electronics configured to measure an output of the strain gauge. In another aspect, the microcontroller of the conditioning module can be in electrical communication with the strain gauge conditioning electronics. In a further aspect, the microcontroller can be configured to communicate a hybrid load cell output to a LED readout or other conventional display means. In this aspect, the microcontroller can be configured to receive the load cell output as described herein during periods when the load applied to the hybrid load cell is less than a predetermined percentage of full scale. The microcontroller can be further configured to receive an output from the strain gauge during periods when the load applied to the hybrid load cell is greater than or equal to the predetermined percentage of full scale. For example, and without limitation, the predetermined percentage of full scale can be between about 5% and 15% of full scale. Thus, it is contemplated that the hybrid load cell output can be equal to the load cell output as described herein until the load applied to the hybrid load cell reaches the predetermined percentage of full scale.

Alternatively, the hybrid load cell can comprise means for attenuating the load cell output as described herein to be less than the output of the strain gauge. It is contemplated that the microcontroller can be configured to receive the load cell output as described herein until the output increases to a predetermined voltage. After the output is greater than or equal to the predetermined voltage, then the microcontroller can be configured to receive the output from the spring gauge.

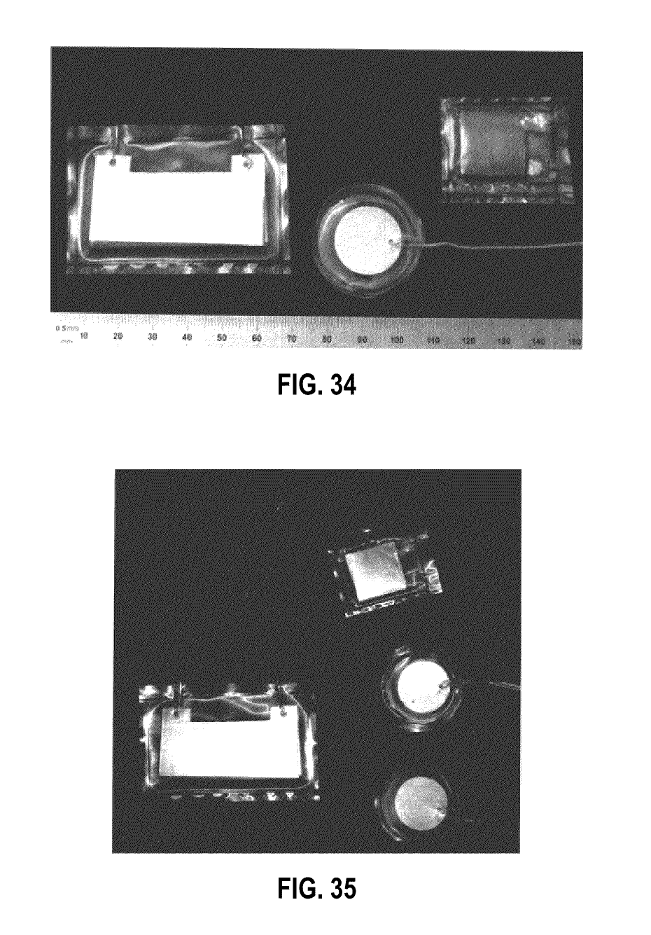

It is contemplated that the hybrid load cell as described herein can maximize the accuracy of load measurements across a wide range of applied loads. In particular, it is contemplated that the accuracy of the hybrid load cell can be substantial consistent from approximately 0% to approximately 90% of full scale. It is further contemplated that the hybrid load cell as described herein can ensure that the zero balance is minimized. In one aspect, the hybrid load cell described herein can have a repeatability of less than about 0.10% at 0.10% of full scale and less than about 0.20% at 0.50% full scale. More preferably, the repeatability of the hybrid load cell can be less than about 0.05% at 0.10% of full scale and less than about 0.10% at 0.50% of full scale. In an additional aspect, the hybrid load cell described herein can have hysteresis of less than 0.01% at 0.10% of full scale and less than about 0.02% at 0.50% of full scale. More preferably, the hysteresis of the hybrid load cell can be less than about 0.002% at 0.10% of full scale and less than about 0.01% at 0.5% of full scale. Referring now to FIGS. 33-35, an alternative embodiment of a force/pressure sensor is illustrated. In this embodiment, the force/pressure sensor can comprises a pliable housing defining an interior cavity and a conductive polymer sensor that is positioned within the interior cavity. In one aspect, the force/pressure sensor further comprises a first electrode and a second electrode that are positioned on opposing sides of the conductive polymer sensor. In one aspect, it is contemplated that the housing of the force/pressure sensor can be hermetically sealed to prevent fluid or gas intrusion there into the interior cavity of the housing.

In another aspect, it is contemplated that the respective first and second electrodes can be substantially the same size as the respective upper and lower surfaces of the opposing sides of the conductive polymer sensor. Optionally, it is contemplated that the respective first and second electrodes can simply be coupled to respective portions of the upper and lower surfaces of the opposing sides of the conductive polymeric sensor.

Further, it is contemplated that the formed force/pressure sensor illustrated in FIGS. 33-35 can be pliable so that the formed force/pressure sensor can be formed or otherwise configured to mirror an underlying surface. In various aspects, the conductive polymeric material in the sensor can be formed thinly—at the surface level, the material remains substantially incompressible and the change in resistance of the material is due to the change in resistance of the surface of the material due to compression that occurs at the molecular level. Thus, it is contemplated that the conductive polymeric material can be formed with a height of ≦0.50 inches, ≦0.45 inches, ≦0.40 inches, ≦0.35 inches, ≦0.30 inches, ≦0.25 inches, ≦0.20 inches, ≦0.15 inches, ≦0.10 inches, ≦0.05 inches, ≦0.04 inches, ≦0.02 inches, and/or ≦0.01 inches.

In operation, the formed force/pressure sensor can be mounted thereon a substantially rigid underlying surface. In this configuration, force applied to the sensor will be read accurately without having to correct for deformation of the underlying surface.

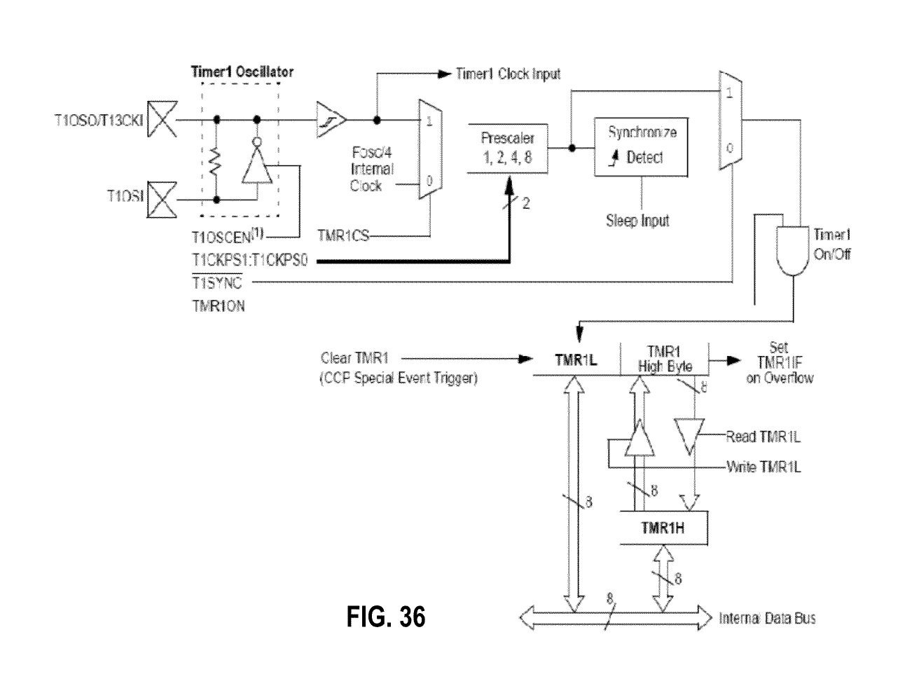

In operation, in one aspect, a power source can be connected to the force/pressure sensor via the first and second electrodes. The conductive polymer sensor between the two electrodes completes the electrical circuit. When a force is applied to the pliable housing, the load is transferred to the first and second electrodes and conductive polymer sensor, which compresses, at a molecular level, the substantially incompressible conductive polymer sensor. As the force increases, the current flow through the conductive polymer sensor from the first electrode to the second electrode increases. In one non-limiting example, the change in voltage across a fixed value shunt resister that is connected in series with the conductive polymer sensor can be converted into an applied force/pressure unit. In one aspect, it is contemplated that the thin profile sensor shown and described with reference to FIGS. 36-38 can use the same conditioning module that the load cell in the housing described above uses.

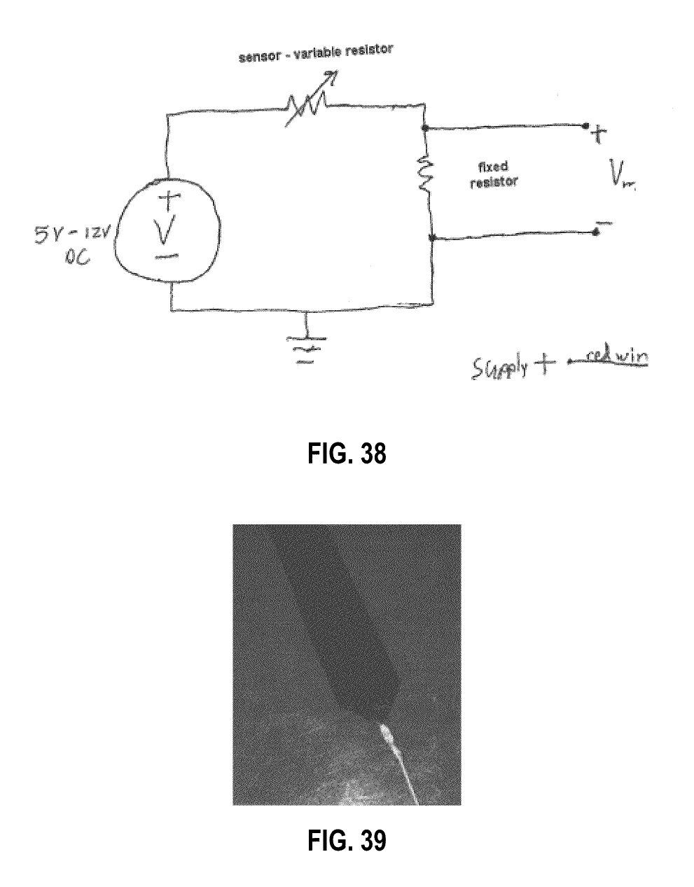

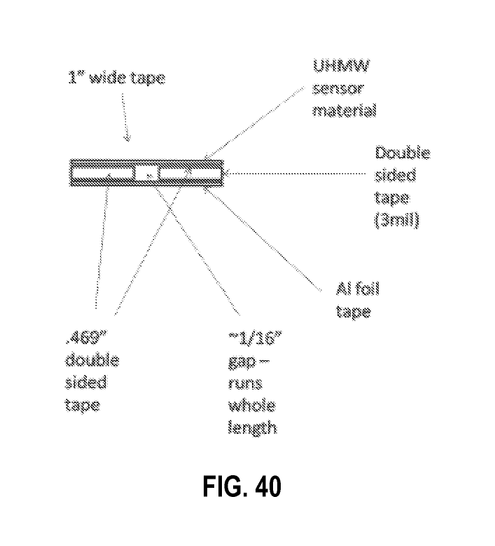

It is also contemplated that the exemplified load cell and the thin profile force/pressure sensor can be used as force/pressure switches, which are configured to act as an electrically switch upon sensing of an applied force. In one aspect, and as shown in FIG. 39, is a perspective photograph of an alternative embodiment of a sensor that is configured to act as a pressure switch. In various aspects, it is contemplated that the force/pressure sensor can be thin-profiled or can be adapted to be selectively mated to a variety of underlying surfaces. For example, at least a portion of a top and/or bottom surface of the force/pressure sensor can have an adhesive fixed thereto so that the force/pressure sensor can be applied like a conventional adhesive tape. In one aspect, and referring to the cross-sectional view of the force/pressure sensor shown in FIG. 40, an exemplary sensor tape can comprise a layer of UHMW sensor material with an adhesive strip attached to at least a portion of the top side of the sensor material and a layer of foil that is positioned underlying the sensor material. In this aspect, the sensor material and the foil can be connected together in a spaced relationship through the use of a plurality of strips of double sided adhesive tape. In one aspect, the strips of double sided adhesive tape can be spaced from each other. A first electrode is coupled to a portion of the sensor material and a second electrode is coupled to the foil. It is contemplated that the first and second electrodes are coupled to the driving electronics as described in more detail above.

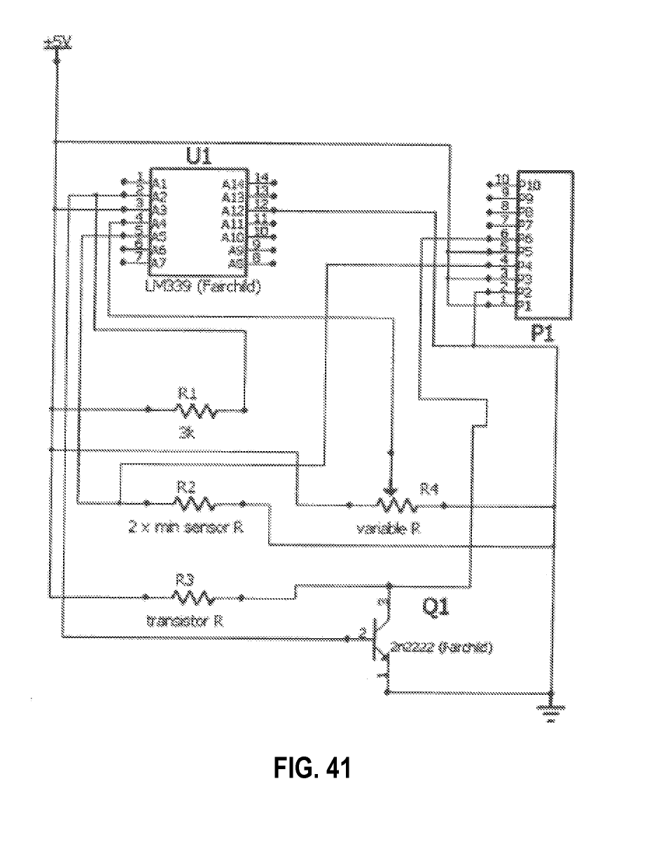

In various aspects it is contemplated that “switch” modalities of the present invention can be used in applications in which is desired to know whether force is acting on the sensor and not necessarily knowing the level of force being applied to the sensor. For example, if it is desired to know whether someone is tampering with a security fence, an operator would apply the sensor tape to the underlying structure (here, the security fence). Any attempted touching, bending, cutting, and the like, thereon the security fence with the sensor tape applied thereto would result in a change of resistance of the sensor tape that can be measured via a comparator circuit such as the comparator circuit that is illustrated in FIG. 41. In one aspect, upon the sensing of a change of resistance indicative of tampering, the comparator circuit can be configured to subsequently activate a relay or other device to interface the sensor tape with an alarm system or the like.

As one skilled in the art will appreciate, the exemplary sensor tape is configured in this example to act as a pressure switch in this case. In another aspect, a similarly configured sensor, which may or may not have an adhesive, can be used as a floor sensor. Thus, it is contemplated that the sensor tape and floor sensor embodiments act as pressure switches, as opposed to load cell and force/pressure sensors described above, which are pressure transducers/pressure sensors that have an analog output corresponding to the applied pressure.

In exemplary aspects, it is contemplated that the disclosed conductive polymer element and electrodes of the load cell can be combined to form a thin membrane sensor. In these aspects, it is contemplated that the thin membrane sensor formed from the conductive polymer element and the first and second electrodes can be provided separately from the load cell housing and, optionally, can be provided with a thin unobtrusive cover.

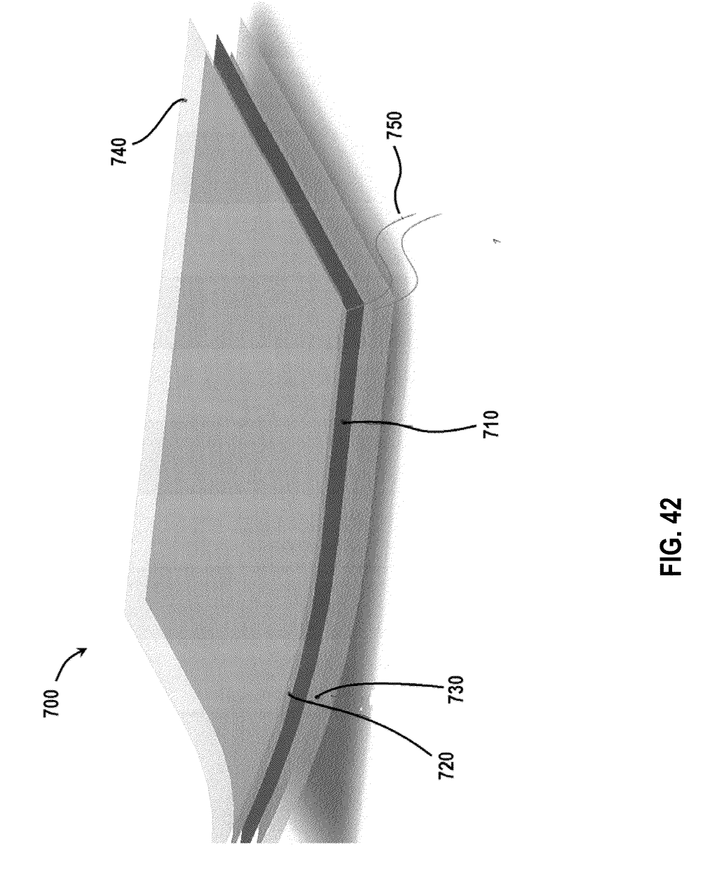

In one aspect, as shown in FIG. 42, the thin membrane sensor 700 can comprise a sheet-like element 710. In this aspect, the sheet-like element can comprise a layer of conductive polymer, such as, for example and without limitation, a layer of UHMWPE having a thickness ranging from between about 0.001 inches to about 0.050 inches, and preferably between about 0.003 inches to about 0.01 inches. However, it is contemplated that any of the polymers herein described can be used to form the thin membrane sensor. It is further contemplated that the thin membrane sensor can further comprise carbon black in a quantity corresponding to a weight percentage ranging from about 0.5% to about 30%, and preferably between about 1% to about 10% of the sheet-like element of the thin membrane sensor.

In another aspect, the sheet-like element can be joined to or otherwise connected with one or more electrodes as described herein to form the thin membrane sensor. Optionally, in one exemplary non-limiting aspect, it is contemplated that a first electrode 720 can be coupled to a top side of the sheet-like element and that a second electrode 730 can be coupled to a bottom side of the sheet-like element. In this aspect, it is contemplated that the first and second electrodes 720, 730 can comprise one of aluminum or copper. In another aspect, it is contemplated that the first and second electrodes 720, 730 can comprise aluminum. In a further aspect, it is contemplated that the thin membrane sensor 700 can optionally be covered with a thin film 740 comprising, for example and without limitation, one of thin polyethylene and silicon. In this aspect, it is contemplated that the thin film 740 can protect the thin membrane sensor 700 while also holding the sheet-like element and the electrodes in a desired orientation.

In another exemplary aspect, the thin film cover 740 of the thin membrane sensor 700 can have a thickness of less than about 0.020 inches, preferably less than about 0.010 inches, and most preferably about 0.005 inches. Similarly, each respective electrode 720, 730 of the thin membrane sensor 700 can have a thickness of less than about 0.020 inches, preferably less than about 0.010 inches, and most preferably about 0.003 inches. In various aspect, the sheet-like element 710 of the thin membrane sensor 700 can have a thickness of less than about 0.050 inches, preferably less than about 0.030 inches, and most preferably about 0.010 inches. In still a further aspect, it is contemplated that wires 750 can be placed in electrical communication with each respective electrode of the thin membrane sensor.

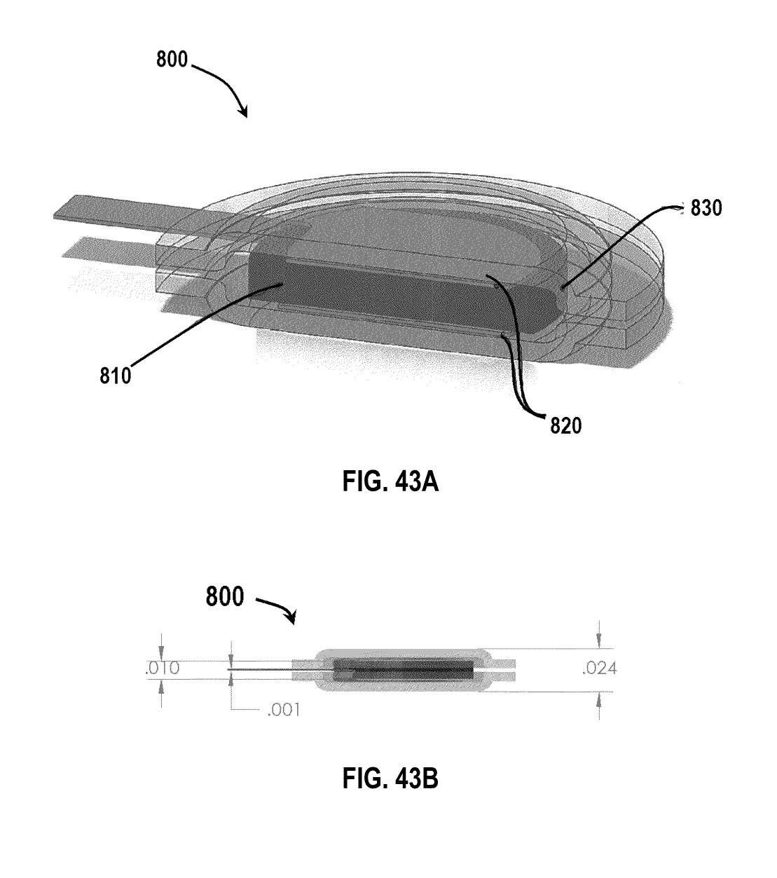

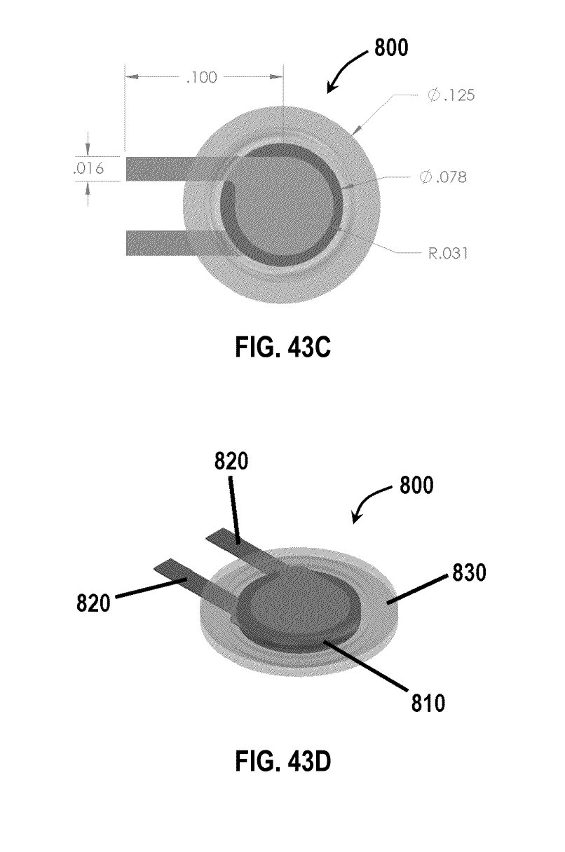

In an additional aspect, and with reference to FIGS. 43A-43D, the sheet-like element 810 of the thin membrane sensor 800 can be selectively dimensioned to have a diameter or width ranging from about 0.10 inches to about 5 inches, preferably between about 0.15 inches to about 4 inches, and most preferably between about 0.2 inches to about 3 inches. In one exemplary aspect, it is contemplated that the sheet-like element 810 can be substantially circular.

In an additional aspect, the thin membrane sensor 800 can optionally comprise one or more electrodes 820 placed or otherwise positioned on at least a portion of a face of the sheet-like element 810. Optionally, in another aspect, it is contemplated that the sheet-like element 810 of the thin membrane sensor 800 can be configured to couple to and overlie at least a portion of a composite polyethylene layer with a desired, elevated contact resistance to carry the electrical signal generated by the thin membrane sensor 800. In this aspect, it is contemplated that the thin membrane sensor 800 can function without the use of the disclosed metallic electrodes 820.

In a further aspect, the thin membrane sensor 800 can comprise a protective housing 830. In an exemplary non-limiting aspect, the thin membrane sensor 800 can comprise a sheet-like element 810 comprising UHMWPE and an amount of carbon black corresponding to between about 1% to about 4%, and preferably about 2% weight by volume of the sheet-like element 810, respective first and second copper electrodes 820 that are connected to or other positioned on at least a portion of the opposing faces of the sheet-like element, and a transparent, polyethylene protective housing 830. In use, it is contemplated that the thin-membrane sensors 800 can have a minimum pressure measurement scale of about 0.5 psi (3.5 Pa) and be configured to measure pressures up to about 6,000 psi (41.4 MPa).

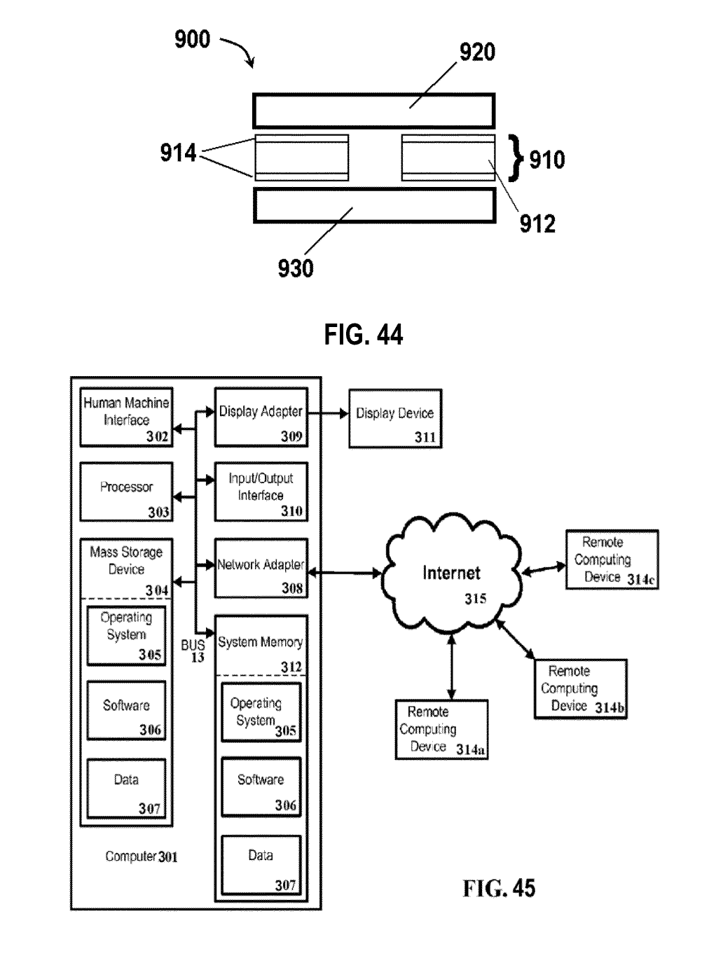

In one exemplary aspect, the sheet-like element of the thin-membrane sensor can have a diameter or width of between about 0.30 inches to about 2.50 inches, and preferably about 1 inch. In this aspect, the sheet-like element can be incorporated into an exemplary sensor tape as described herein. In one aspect, as shown in FIG. 44, the sensor tape 900 can comprise two spacers 910 positioned between and connected thereto the sheet-like element 920 and a single electrode 830, such as, for example and without limitation, a copper electrode. In a further aspect, it is contemplated that the spacers 910 can each comprise a spacer material 912, such as Teflon, positioned between first and second pieces of two-sided adhesive tape 914. In use, it is contemplated that the sensor tape 900 can be wrapped around a cylindrical, or other rounded, surface.

During and/or after manufacture of these thin membrane sensors, it is contemplated that the sheet-like members can be initially formed into larger dimensions before being cut to a desired size and shape. For example, it is contemplated that the sheet-like element can be cut to a desired length, width, or thickness prior to use of the thin membrane sensor.

It is contemplated that the sensors disclosed herein can have a wide range of mechanical and electrical properties, depending on the particular polymers and other materials that are selected for a given application. Thus, for example, the elastic modulus, elongation, elasticity, wear and impact resistance, frictional coefficients, temperature resistance, battery life, and signal-to-noise ratio associated with any of the disclosed sensors can vary significantly depending on the particular materials used.

It is further contemplated that the disclosed sensors can be configured to measure a pressure ranging from under 1 psi (6.9 Pa) to above 2,000 psi (13.8 MPa). It is further contemplated that a sensor with a 1,000 lbf measurement capacity that is made as described herein can be configured to measure an applied force as low as about 0.04 lbf (20 g). Additionally, it is contemplated that the disclosed sensors can be configured to have an overload limit of about 10,000 psi (69 MPa). It is still further contemplated that the power usage associated with the disclosed sensors can range from 1 μA to about 10 μA for a lower-power sensor and from about 1 mA to about 20 mA for a low-noise sensor. It is still further contemplated that the excitation voltage associated with the disclosed sensors can range from about 10 mV to over about 20 V, such that the commonly used 3.3 V excitation voltage is appropriate for the disclosed sensors. Further, it is contemplated that the disclosed sensors can be configured to function accurately at temperatures ranging from about −40° C. to about 260° C.

In one example, a contact sensor can comprise a data acquisition terminal and a polymeric body having a contact surface configured to receive a load. It is contemplated that the contact surface of the polymeric body can have at least one conductive portion that is in communication with the data acquisition terminal. It is further contemplated that the conductive portion of the contact surface, during application of the load, can comprise means for producing an output signal indicative of the change in electrical resistance experienced across the contact surface at the least one conductive portion. It is still further contemplated that the output signal can correspond to variations in the received load on the contact surface.

Optionally, the exemplary contact sensor can further comprise at least one electrode coupled to at least a portion of each conductive portion of the contact surface. It is contemplated that the at least one electrode can comprise a pair of opposed electrodes. It is further contemplated that the polymeric body can be positioned therebetween the pair of opposed electrodes.

Optionally, the at least one conductive portion of the exemplary contact sensor can comprise a plurality of selected spaced conductive portions. It is contemplated that these selected spaced conductive portions can define an array of sensing points.

In use, the output signal of the exemplary contact sensor can be indicative of the change in electrical resistance experienced across the contact surface at least one sensing point. It is contemplated that the output signal produced by each sensing point can correspond to variations in the applied load.

Optionally, the exemplary contact sensor can further comprise an electrically conductive joint element. It is contemplated that the load can be applied to the contact surface by a portion of the electrically conductive joint element.

Optionally, the conductive portions of the contact surface can form conductive stripes extending the substantial length of the contact surface.

Optionally, the conductive portions of the contact surface can form a plurality of dots spaced along the contact surface.

It is contemplated that the data acquisition terminal can be programmed to measure the current at each sensing point of the array of sensing points. It is further contemplated that the data acquisition terminal can be programmed to process the current measurements at at least one sensing point to determine the pressure that is applied at each sensing point.

It is contemplated that the polymeric body can comprise a substantially inflexible composite material. It is further contemplated that the substantially inflexible composite material can comprise an at least partially conductive polymeric material.

It is contemplated that the conductive portion of each polymeric body can be formed from a pressure sensitive conductive composite material that comprises an electrically conductive filler and a polymeric material. It is further contemplated that the non-conductive portion of each polymeric body can comprise a polymeric material. It is still further contemplated that the polymeric material used in the conductive and non-conductive portions can be the same polymeric material. It is still further contemplated that the polymeric material can be a thermoformable polymer. It is still further contemplated that polymeric material can be selected from a group consisting of: ultra high molecular weight polyethylene (UHMWPE), high density polyethylene (HDPE), polyphenylene sulfide (PPS), low density polyethylene (LDPE), or polyoxymethylene copolymer (POM).

It is contemplated that the exemplary contact sensor can comprise a desired amount of conductive filler. It is further contemplated that the desired amount of conductive filler can range from about 0.1% to about 20% by weight of the pressure sensitive composite material. It is still further contemplated that the desired amount of conductive filler can range from about 1% to about 15% by weight of the pressure sensitive composite material. It is still further contemplated that the desired amount of conductive filler can range from about 5% to about 12% by weight of the pressure sensitive composite material. It is contemplated that the conductive filler of the exemplary contact sensor can comprise carbon black. It is further contemplated that the pressure sensitive composite material of can further comprise ceramic fillers, aluminum oxide, zirconia, calcium, silicon, fibrous fillers, carbon fibers, glass fibers, and/or organic fillers.

It is contemplated that the polymeric body of the exemplary contact sensor can be formed into the shape of at least a portion of an artificial joint bearing. It is further contemplated that the contact surface of the exemplary contact sensor can extend therein the polymeric body to a depth ranging from about 50 nm to about 1000 nm.

In another example, a contact sensor system can comprise a data acquisition terminal and a surgical insert defining a contact surface configured to receive a load applied by an electrically conductive joint element. It is contemplated that the contact surface can have selected spaced conductive portions. It is further contemplated that the selected spaced conductive portions can define an array of sensing points that are in communication with the data acquisition terminal It is still further contemplated that the surgical insert can be configured for insertion therein a selected joint within the body of a subject. It is still further contemplated that the conductive portions of the contact surface, during application of the load, can comprise means for producing an output signal indicative of the change in electrical resistance experienced across the contact surface at at least one sensing points. It is still further contemplated that the output signal produced by each sensing point can correspond to variations in the load between the electrically conductive joint element and the contact surface.

It is contemplated that the selected joint for insertion of the surgical insert can comprise one of a knee joint, a hip joint, a shoulder joint, an ankle joint, and a spinal joint. It is further contemplated that the surgical insert can comprise one of a tibial insert, a femoral insert, a patellar insert, an acetabular insert, a scapular insert, a humeral insert, a talar insert, and a vertebral insert.

It is still further contemplated that contact sensor of the exemplary system can extend therein the surgical insert to a depth ranging from about 50 nm to about 1000 nm.

FIG. 45 is a block diagram illustrating an exemplary operating environment for performing the disclosed methods and portions thereof. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

The present methods and systems can be operational with numerous other general purpose or special purpose computing system environments or configurations. Examples of well known computing systems, environments, and/or configurations that can be suitable for use with the system and method comprise, but are not limited to, personal computers, server computers, laptop devices, hand-held electronic devices, vehicle-embedded electronic devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components. The disclosed system and method can be described in the general context of computer-executable instructions, such as program modules, being executed by one or more computers or other devices. Generally, program modules comprise computer code, routines, programs, objects, components, data structures, etc. that performs particular tasks or implement particular abstract data types. In one aspect, the program modules can comprise a system control module. The disclosed method can also be practiced in grid-based and distributed computing environments where tasks are performed by remote processing devices that are linked through a communications network. In a distributed computing environment, program modules can be located in both local and remote computer storage media including memory storage devices.

Further, one skilled in the art will appreciate that the system and method disclosed herein can be implemented via a general-purpose computing device in the form of a computer 300. As schematically illustrated in FIG. 45, the components of the computer 300 can comprise, but are not limited to, one or more processors or processing units 303, a system memory 312, and a system bus 313 that couples various system components including the processor 303 to the system memory 312.

The system bus 313 represents one or more of several possible types of bus structures, including a memory bus or memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI) bus also known as a Mezzanine bus. The bus 313, and all buses specified in this description can also be implemented over a wired or wireless network connection and each of the subsystems, including the processor 303, a mass storage device 304, an operating system 305, load cell software 306, load cell and/or treatment data 307, a network adapter 308, system memory 312, an Input/Output Interface 310, a display adapter 309, a display device 311, and a human machine interface 302, can be contained within one or more remote computing devices 314a,b,c at physically separate locations, connected through buses of this form, in effect implementing a fully distributed system.

The computer 300 typically comprises a variety of computer readable media. Exemplary readable media can be any available media that is accessible by the computer 300 and comprises, for example and not meant to be limiting, both volatile and non-volatile media, removable and non-removable media. The system memory 312 can comprise computer readable media in the form of volatile memory, such as random access memory (RAM), and/or non-volatile memory, such as read only memory (ROM). The system memory 312 typically contains data such as pressure and/or hysteresis data 307 and/or program modules such as operating system 305 and load cell module software 306 that are immediately accessible to and/or are presently operated on by the processing unit 303.

In another aspect, the computer 300 can also comprise other removable/non-removable, volatile/non-volatile computer storage media. By way of example, FIG. 45 illustrates a mass storage device 304 which can provide non-volatile storage of computer code, computer readable instructions, data structures, program modules, and other data for the computer 300. For example and not meant to be limiting, a mass storage device 304 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 304, including by way of example, an operating system 305 and load cell module software 306. Each of the operating system 305 and load cell module software 306 (or some combination thereof) can comprise elements of the programming and the load cell module software 306. Pressure and/or hysteresis data 307 can also be stored on the mass storage device 304. Pressure and/or hysteresis data 307 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple systems.

In another aspect, the user can enter commands and information into the computer 300 via an input device (not shown). Examples of such input devices comprise, but are not limited to, a keyboard, pointing device (e.g., a “mouse”), a microphone, a joystick, a scanner, tactile input devices such as gloves, and other body coverings, and the like These and other input devices can be connected to the processing unit 303 via a human machine interface 302 that is coupled to the system bus 313, but can be connected by other interface and bus structures, such as a parallel port, game port, an IEEE 1394 Port (also known as a Firewire port), a serial port, or a universal serial bus (USB).

In yet another aspect, a display device 311 can also be connected to the system bus 313 via an interface, such as a display adapter 309. It is contemplated that the computer 300 can have more than one display adapter 309 and the computer 300 can have more than one display device 311. For example, a display device can be a monitor, an LCD (Liquid Crystal Display), or a projector. In addition to the display device 311, other output peripheral devices can comprise components such as a printer (not shown) which can be connected to the computer 300 via Input/Output Interface 310.

The computer 300 can operate in a networked environment using logical connections to one or more remote computing devices 314a,b,c. By way of example, a remote computing device can be a personal computer, portable computer, a server, a router, a network computer, a peer device or other common network node, and so on. Logical connections between the computer 300 and a remote computing device 314a,b,c can be made via a local area network (LAN) and a general wide area network (WAN). Such network connections can be through a network adapter 308. A network adapter 308 can be implemented in both wired and wireless environments. Such networking environments are conventional and commonplace in offices, enterprise-wide computer networks, intranets, and the Internet 315.

For purposes of illustration, application programs and other executable program components such as the operating system 305 are illustrated herein as discrete blocks, although it is recognized that such programs and components reside at various times in different storage components of the computing device 300, and are executed by the data processor(s) of the computer. An implementation of load cell software 306 can be stored on or transmitted across some form of computer readable media. Computer readable media can be any available media that can be accessed by a computer. By way of example and not meant to be limiting, computer readable media can comprise “computer storage media” and “communications media.” “Computer storage media” comprise volatile and non-volatile, removable and non-removable media implemented in any method or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

In various aspects, it is contemplated that the methods and systems described herein can employ Artificial Intelligence techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. expert inference rules generated through a neural network or production rules from statistical learning).

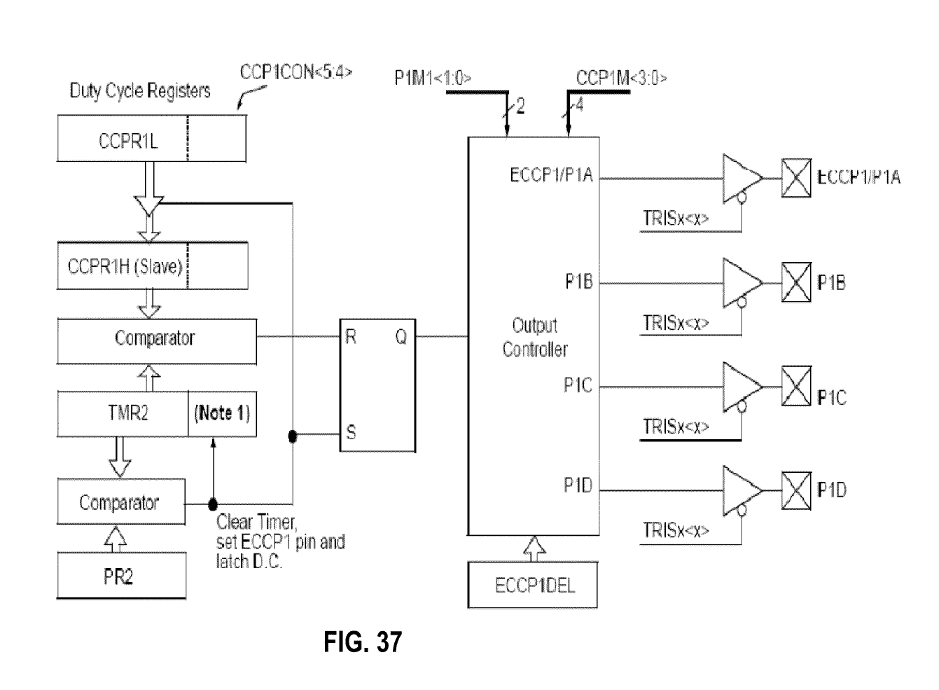

In one aspect, and referring to FIGS. 36-37, the conversion of the load cell output can be timed by the controller. In this aspect, it is contemplated that a hardware, or optionally software timer, can be loaded with a “rollover” value, such that, when it has counted a desired time interval, the timer will start the A/D converter and resets itself to zero to repeat the process. In one exemplary aspect, it is contemplated that a new conversion starts every 125 millisecond for an overall 8 KHz sampling rate.

In one example, and referring to FIG. 36, the TIMER1 of the conditioning module can be wire to the second “Enhanced Capture, Control and PWM” module (the “ECCP2”). Referring to a PIC19F8722 Family Datasheet, an A/D conversion can be started by the special event trigger of the ECCP2 module. When the trigger occurs, the GO/DONE bit will be set, starting the A/D acquisition and conversion and the Timer1 (or Timer3) counter will be reset to zero. Timer 1 (or Timer3) is reset to automatically repeat the A/D acquisition period with minimal software overhead. In one aspect, the prescaler is loaded as appropriate and the CP Special Event Trigger is set to trip at a 125 millisecond interval. Simultaneously with the start of the A/D conversion, the timer is reset. The D/A output is latched to the same timer.

Referring now to FIG. 37, showing a block diagram of the ECCP1 system, which, like the ECCP2 (which trips the A/D conversion) is also locked to TIMER1. Here, shortly after TIMER1 rollover, the value in the “comparator” is equal to what is in TIMER1. Thus, with the proper value loaded in the comparator, the ECCP1/P 1A pin will toggle at an interval precisely behind the actual taking of the A/D conversion reading.

In another aspect, it is contemplated that an A/D reading for pressure is taken and an A/D reading for temperature is taken. The pressure A/D value can then be run through a lowpass filter algorithm to remove noise and set an upper frequency limit on response. That pressure result can be then run through a set of pressure lookup tables. The temperature A/D value can be run though a set of temperature lookup tables to provide a temperature correction factor. After the temperature correction factor is calculated, a subtraction of any value for “zero calibration” is accomplished to insure that “zero” is the actual “zero” point of the load cell. This “zero cal” value can be stored in the EEPROM of the device and its value can be retained though a power cycle of the device. It is contemplated that this “zero cal” value is not retained though a reprogramming activity.

Referring now to FIG. 38, an exemplary schematic for a simplified electrical circuit for the load cell is illustrated. In this aspect, a voltage is applied to the conductive polymeric sensor, which is the variable resistor in the circuit diagram, and a shunt resister in series. The shunt resister has a fixed resistance and the change in voltage across the shunt resister can be measured when force is applied to the conductive polymeric sensor. As one skilled in the art will appreciate, the change in voltage can be converted into force/pressure engineering units. In one aspect, the conditioning module can comprise the source of the voltage and the shunt resister.

These and other modifications and variations to the present invention may be practiced by those of ordinary skill in the art, without departing from the spirit and scope of the present invention, which is more particularly set forth in the appended claims. In addition, it should be understood that aspects of the various aspects may be interchanged either in whole or in part. Furthermore, those of ordinary skill in the art will appreciate that the foregoing description is by way of example only, and is not intended to limit the invention so further described in such appended claims.

1. A contact sensor, comprising:
a data acquisition terminal; and
a polymeric body having a contact surface configured to receive a load, the contact surface having at least one conductive portion that is in communication with the data acquisition terminal, wherein the conductive portion of the contact surface, during application of the load, comprises means for producing an output signal indicative of the change in electrical resistance experienced across the contact surface at the at least one conductive portion,
wherein the output signal corresponds to variations in the received load on the contact surface.

2. The contact sensor of claim 1, further comprising at least one electrode coupled to at least a portion of each conductive portion.

3. The contact sensor of claim 2, wherein the at least one electrode comprises a pair of opposed electrodes, and wherein the polymeric body is positioned therebetween the pair of opposed electrodes.

4. The contact sensor of claim 1, wherein the at least one conductive portion comprised a plurality of selected spaced conductive portions, and wherein the selected spaced conductive portions define an array of sensing points.

5. The contact sensor of claim 4, wherein the output signal is indicative of the change in electrical resistance experienced across the contact surface at at least one sensing point, wherein the output signal produced by each sensing point corresponds to variations in the applied load.

6. The contact sensor of claim 5, further comprising an electrically conductive joint element, wherein the load is applied to the contact surface by a portion of the electrically conductive joint element.

7. The contact sensor of claim 5, wherein the conductive portions of the contact surface form conductive stripes extending the substantial length of the contact surface.

8. The contact sensor of claim 5, wherein the conductive portions of the contact surface form a plurality of dots spaced along the contact surface.

9. The contact sensor of claim 5, wherein the data acquisition terminal is programmed to measure the current at each sensing point of the array of sensing points.

10. The contact sensor of claim 5, wherein the data acquisition terminal is programmed to process the current measurements at at least one sensing point to determine the pressure that is applied at each sensing point.

11. The contact sensor of any of claim 1, wherein the polymeric body comprises a substantially inflexible composite material.

12. The contact sensor of claim 11, wherein the substantially inflexible composite material comprises an at least partially conductive polymeric material.

13. The contact sensor of claim 1, wherein the conductive portion of each polymeric body is formed from a pressure sensitive conductive composite material that comprises an electrically conductive filler and a polymeric material.

14. The contact sensor of claim 13, wherein the non-conductive portion of each polymeric body comprises a polymeric material.

15. The contact sensor of claim 13, wherein the polymeric material used in the conductive and non-conductive portions are the same polymeric material.

16. The contact sensor of claim 13, wherein the polymeric material is a thermoformable polymer.

17. The contact sensor of claim 13, wherein the polymeric material is selected from a group consisting of: ultra high molecular weight polyethylene (UHMWPE), high density polyethylene (HDPE), polyphenylene sulfide (PPS), low density polyethylene (LDPE), or polyoxymethylene copolymer (POM).

18. The contact sensor of claim 13, wherein a desired amount of conductive filler can range from about 0.1% to about 20% by weight of the pressure sensitive composite material.

19. The contact sensor of claim 13, wherein a desired amount of conductive filler can range from about 1% to about 15% by weight of the pressure sensitive composite material.

20. The contact sensor of claim 13, wherein a desired amount of conductive filler can range from about 5% to about 12% by weight of the pressure sensitive composite material.

21. (canceled)

22. The contact sensor of claim 13, wherein the pressure sensitive composite material further comprises ceramic fillers, aluminum oxide, zirconia, calcium, silicon, fibrous fillers, carbon fibers, glass fibers, and/or organic fillers.

23. The contact sensor of claim 13, wherein the polymeric body can be formed into the shape of at least a portion of an artificial joint bearing.

24. The contact sensor of claim 13, wherein the contact surface extends therein the polymeric body to a depth ranging from about 50 nm to about 1000 nm.

25. A contact sensor system, comprising:
a data acquisition terminal; and
a surgical insert defining a contact surface configured to receive a load applied by an electrically conductive joint element, the contact surface having selected spaced conductive portions, wherein the selected spaced conductive portions define an array of sensing points that are in communication with the data acquisition terminal, wherein the surgical insert is configured for insertion therein a selected joint within the body of a subject, and wherein the conductive portions of the contact surface, during application of the load, comprises means for producing an output signal indicative of the change in electrical resistance experienced across the contact surface at at least one sensing points,
wherein the output signal produced by each sensing point corresponds to variations in the load between the electrically conductive joint element and the contact surface.

26. The contact sensor of claim 19, wherein the selected joint comprises one of a knee joint, a hip joint, a shoulder joint, an ankle joint, and a spinal joint.

27. The contact sensor of claim 20, wherein the surgical insert comprises one of a tibial insert, a femoral insert, a patellar insert, an acetabular insert, a scapular insert, a humeral insert, a talar insert, and a vertebral insert.

28. The contact sensor of claim 19, wherein the contact surface extends therein the surgical insert to a depth ranging from about 50 nm to about 1000 nm.
